(19) United States
(12) Patent Application Publication
(10) Pub. No.: US 2024/0042046 A1
(43) Pub. Date: Feb. 8, 2024

(54) METHODS AND COMPOUNDS FOR MODULATING HUNTINGTON'S DISEASE

(71) Applicant: Design Therapeutics, Inc., Carlsbad, CA (US)

(72) Inventors: Chengzhi ZHANG, Carlsbad, CA (US); Abhijit BHAT, Carlsbad, CA (US); Jarod WAYBRIGHT, Carlsbad, CA (US); Hannah NOURIE, Carlsbad, CA (US); Fei YANG, Carlsbad, CA (US)

(21) Appl. No.: 18/480,117

(22) Filed: Oct. 3, 2023

Related U.S. Application Data
(63) Continuation of application No. PCT/US2023/017201, filed on Mar. 31, 2023.
(60) Provisional application No. 63/326,625, filed on Apr. 1, 2022.
(60) Provisional application No. 63/482,670, filed on Feb. 1, 2023.

(51) Int. Cl. A61K 47/64; A61K 47/54; A61P 25/28

(52) U.S. Cl. CPC A61K 47/64; A61K 47/545; A61P 25/28

(57) ABSTRACT

The present disclosure relates to transcription modulator molecules having a first terminus, a second terminus, and an oligomeric backbone and methods for treating Huntington's disease.

### CROSS REFERENCE TO RELATED APPLICATIONS

This application is a continuation of International Application No.: PCT/US2023/017201, filed Mar. 31, 2023, which claims the benefit of U.S. Application No. 63/326,625, filed Apr. 1, 2022, and U.S. Application No. 63/482,670, filed Feb. 1, 2023, each of which are hereby incorporated by reference in their entirety.

### FIELD OF THE DISCLOSURE

Disclosed herein are new chimeric heterocyclic polyamide compounds and compositions and their application as pharmaceuticals for the treatment of disease. Methods to modulate the expression of a target gene comprising the CAG trinucleotide repeat sequence in a subject are also provided for the treatment of diseases such as Huntington's disease (“HD”).

### BACKGROUND OF THE DISCLOSURE

Huntington's disease (“HD”) was first identified in the late 19^{th} century as an autosomal dominant, neurodegenerative disorder. The symptoms of HD, which include a range of movement, cognitive and psychiatric disorders, generally appear in adulthood. HD is associated with the presence of the CAG trinucleotide repeat sequence in the Htt gene, which codes for a protein termed huntingtin. Subjects with more than about 36 trinucleotide repeat sequences generally present with symptoms of HD, with a larger number of trinucleotide repeat sequences associated with an earlier onset of symptoms. Pathology stems from a cascade of steps: production of poly-Q huntingtin, followed by fragmentation of the elongated huntingtin into smaller peptides, which bind together and accumulate in neurons. The effects of this cascade are pronounced in the basal ganglia and cortex of the brain.

Huntington's disease-like syndrome refers to a group of ailments whose symptoms are similar to those of Huntington's disease, but which lack the characteristic mutation in the Htt gene. Huntington's disease-like 2 syndrome (“HDL2”) is associated with a count of about 40 or more CAG trinucleotide repeat sequences in the junctophilin 3 (Jph3) gene. HDL2 is a genetic disorder that has been seen in subjects with African lineage. Age of onset is inversely correlated with the number of trinucleotide repeat sequences. Symptoms of this syndrome include dystonia and chorea (uncontrolled movements), emotional disruptions, dysarthria, bradykinesia, inability to incorporate new learning, and difficulty in making decisions. Life expectancy can range from a few years post diagnosis to over a decade. The current theory holds that a poly-Q protein that is coded by the Jph3 gene forms aggregates in neuronal cells that is responsible for the pathology of the disease. However, evidence suggesting toxic gain-of-function of mRNA has also been uncovered, indicating a possible dual pathway for pathology.

In some embodiments, the mechanism set forth above provides opportunity for an effective treatment for a disease or disorder which is characterized by the presence of an excessive count of CAG trinucleotide repeat sequences in a target gene. In some embodiments, the pathology of the disease or disorder is due to the presence of mRNA containing an excessive count of CAG trinucleotide repeat sequences. In some embodiments, the pathology of the disease or disorder is due to the presence of a translation product containing an excessive count of glutamine amino acid residues. In some embodiments, the pathology of the disease or disorder is due to a loss of function in the translation product. In some embodiments, the pathology of the disease or disorder is due to a gain of function in the translation product. In some embodiments, the pathology of the disease or disorder can be alleviated by increasing the rate of transcription of the defective gene. In some embodiments, the pathology of the disease or disorder can be alleviated by decreasing the rate of transcription of the defective gene.

### SUMMARY OF THE DISCLOSURE

This disclosure utilizes regulatory molecules present in cell nuclei that control gene expression. Eukaryotic cells provide several mechanisms for controlling gene replication, transcription, and/or translation. Regulatory molecules that are produced by various biochemical mechanisms within the cell can modulate the various processes involved in the conversion of genetic information to cellular components. Several regulatory molecules are known to modulate the production of mRNA and, if directed to the target gene (such as, Htt), would modulate the production of the target gene mRNA that causes diseases such as, for example, Huntington's disease or Huntington's disease-like syndrome, and thus reverse the progress of these diseases.

Provided herein are compounds and methods for recruiting a regulatory molecule into close proximity to the target gene comprising a CAG trinucleotide repeat sequence. The compounds disclosed herein contain: (a) a DNA binding moiety that will selectively bind to the target gene, linked to (b) a recruiting moiety that will bind to a regulatory molecule. Without being bound by theory, the compounds may counteract the expression of defective target gene in the following manner:
(1) The DNA binding moiety can bind selectively the characteristic CAG trinucleotide repeat sequence of the target gene;
(2) The recruiting moiety, linked to the DNA binding moiety, can thus be held in proximity to the target gene;
(3) The recruiting moiety, now in proximity to the target gene, can recruit the regulatory molecule into proximity with the gene; and
(4) The regulatory molecule can modulate expression of the target gene and therefore counteract the expression of defective mRNA, by direct interaction with the gene.

The DNA binding moiety can bind selectively the characteristic CAG trinucleotide repeat sequence of for example, Htt. The recruiting moiety, linked to the DNA binding moiety, will thus be held in proximity to the target gene; will recruit the regulatory molecule into proximity with the gene; and the regulatory molecule will modulate expression, and therefore counteract the production of defective target gene by direct interaction with the target gene. This mechanism may provide an effective treatment for HD, which is caused by the expression of defective Htt, where correction of the expression of the defective target gene thus represents an effective method for the treatment for these diseases.

The disclosure further provides for DNA binding moieties that selectively bind to one or more copies of the CAG trinucleotide repeat that are characteristic of the defective target gene. Selective binding of the DNA binding moiety to the target gene, made possible due to the high CAG count associated with the defective target gene, directs the recruiting moiety into proximity of the gene, and recruits the regulatory molecule into position to modulate gene transcription.

The DNA binding moiety comprises a polyamide segment that will bind selectively to the target CAG sequence. Polyamides designed by for example Dervan (U.S. Pat. Nos. 9,630,950 and 8,524,899) and others can selectively bind to selected DNA sequences. These polyamides sit in the minor groove of double helical DNA and form hydrogen bonding interactions with the Watson-Crick base pairs. Polyamides that selectively bind to particular DNA sequences can be designed by linking monoamide building blocks according to established chemical rules. One building block is provided for each DNA base pair, with each building block binding noncovalently and selectively to one of the DNA base pairs: A/T, T/A, G/C, and C/G. Following this guideline, trinucleotides bind to molecules with three amide units, i.e. tri-amides. In general, these polyamides can orient in either direction of a DNA sequence.

In principle, longer DNA sequences can be targeted with higher specificity and/or higher affinity by combining a larger number of monoamide building blocks into longer polyamide chains. Ideally, the binding affinity for a polyamide would simply be equal to the sum of each individual monoamide/DNA base pair interaction. In practice, however, due to the geometric mismatch between the fairly rigid polyamide and DNA structures, longer polyamide sequences do not bind to longer DNA sequences as tightly as would be expected from a simple additive contribution. The geometric mismatch between longer polyamide sequences and longer DNA sequences induces an unfavorable geometric strain that subtracts from the binding affinity that would be otherwise expected.

The disclosure provides for transcription modulator molecules that comprise a DNA binding moiety (for example a polyamide comprising multi-amine subunits) that is connected by a spacer (for example a linker moiety or oligomeric backbone) to the protein binding moiety. The spacer can alleviate the geometric strain that would otherwise decrease binding affinity of a larger polyamide sequence.

Disclosed herein are compounds that comprise a polyamide moiety that can bind to one or more copies of the CAG trinucleotide repeat sequence, and can modulate the expression of a target gene comprising a CAG trinucleotide repeat sequence. Treatment of a subject with these compounds will modulate expression of the defective target gene, and this can reduce the occurrence, severity, or frequency of symptoms associated with disease. Certain compounds disclosed herein will provide higher binding affinity and selectivity than has been observed previously for this class of compounds.

It should be understood, however, that the detailed description and the specific examples, while indicating specific embodiments, are given by way of illustration only, since various changes and modifications within the spirit and scope of the instant disclosure will become apparent to those skilled in the art from this detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

The disclosure provides for transcription modulator molecules that comprise a DNA binding moiety (for example, a polyamide comprising multi-amine subunits) connected by spacers (for example, a linker moiety or oligomeric backbone) to the protein binding moiety. The spacers can alleviate the geometric strain that would otherwise decrease binding affinity of a larger polyamide sequences.

Treatment of a subject with these compounds will modulate the expression of the defective target gene, and this can reduce the occurrence, severity, or frequency of symptoms associated with genetic disease (such as for example HD). The compounds described herein recruit the regulatory molecule to modulate the expression of the defective target gene and effectively treat and alleviate the symptoms associated with diseases.

Compounds

The compounds disclosed herein are transcription modulator molecules. They possess useful activity for modulating the transcription of a target gene having one or more CAG repeats (e.g., Htt), and may be used in the treatment or prophylaxis of a disease or condition in which the target gene plays an active role. Thus, in broad aspects, some embodiments also provide pharmaceutical compositions comprising one or more compounds disclosed herein together with a pharmaceutically acceptable carrier, as well as methods of making and using the compounds and compositions.

In an aspect, provided herein is a transcription modulator molecule having a first terminus, a second terminus, and a linker moiety, wherein:
(a) the first terminus comprises a DNA-binding moiety capable of binding a nucleotide repeat comprising CAG;
(b) the second terminus comprises a protein-binding moiety capable of binding to a regulatory molecule that modulates expression of a gene having the nucleotide repeat; and
(c) the oligomeric backbone links the first terminus and the second terminus.

First Terminus—DNA Binding Moiety

The first terminus interacts and binds with the gene, particularly with the minor grooves of the CAG sequence. In an aspect, the molecules disclosed herein provide a polyamide sequence for interaction of a single polyamide subunit to each base pair in the CAG repeat sequence. In some embodiments, the molecules provide a turn component (e.g., aliphatic amino acid moiety), in order to enable hairpin binding of the molecule to the CAG, in which each nucleotide pair interacts with two subunits of the polyamide. In some embodiments, one or more of the polyamide backbone carbonyl groups (C═O), is replaced with an oxetane. In some embodiments, at least one of the polyamide backbone carbonyl groups is replaced with an oxetane.

In some embodiments, each subunit comprises a moiety that is independently chosen from a heterocycle and an aliphatic chain.

In some embodiments, the aliphatic chain is a C₁-C₆ straight chain aliphatic chain. In some embodiments, the aliphatic chain has structural formula —(CH₂)ₘ—, for m chosen from 1, 2, 3, 4, and 5. In some embodiments, the aliphatic chain is —CH₂CH₂—.

In some embodiments, the heterocycle is a monocyclic heterocycle. In some embodiments, the heterocycle is a monocyclic 5-membered heterocycle. In some embodiments, each heterocycle contains a heteroatom independently chosen from N, O, or S. In some embodiments, each heterocycle is independently chosen from pyrrole, imidazole, thiazole, oxazole, thiophene, and furan.

In some embodiments, the DNA-binding moiety comprises —NH-Q-C(O)—, wherein Q is an optionally substituted C₆-C₁₀ arylene, optionally substituted 4 to 10-membered heterocyclene, optionally substituted 5 to 10-membered heteroarylene group, or an optionally substituted alkylene group.

In some embodiments, the DNA-binding moiety comprises at least three aromatic carboxamide moieties selected to correspond to the nucleotide repeat sequence CAG and at least one aliphatic amino acid residue chosen from the group consisting of glycine, β-alanine, γ-aminobutyric acid, 2,4-diaminobutyric acid, and 5-aminovaleric acid. In some embodiments, the DNA-binding moiety comprises one or more subunits selected from the group consisting of optionally substituted N-methylpyrrole, optionally substituted N-methylimidazole, β-alanine (β), and γ-aminobutyric acid. In some embodiments, the DNA-binding moiety comprises at least one γ-aminobutyric acid.

In some embodiments, the DNA-binding moiety comprises a polyamide of one or more of the following subunits selected from

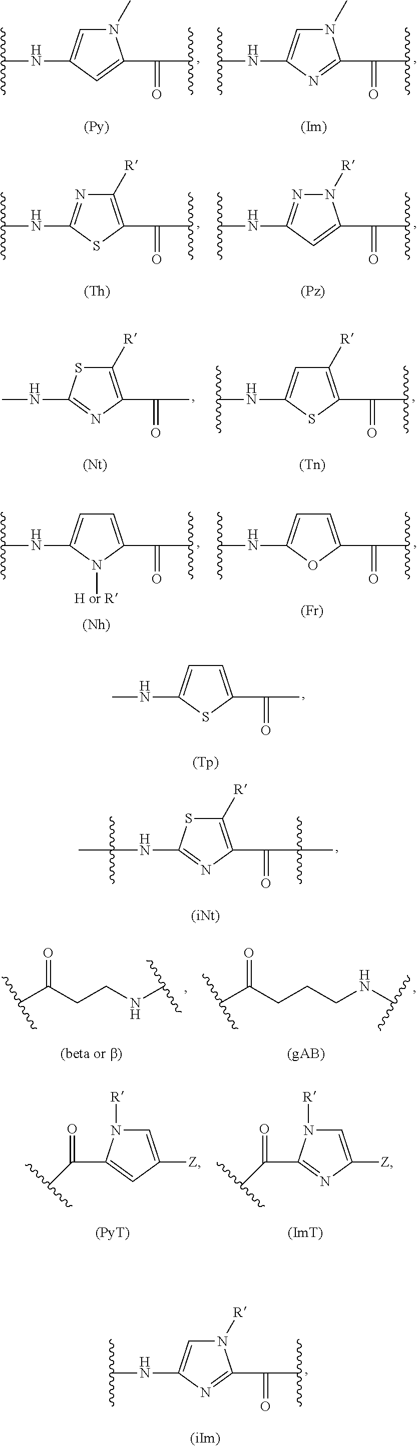

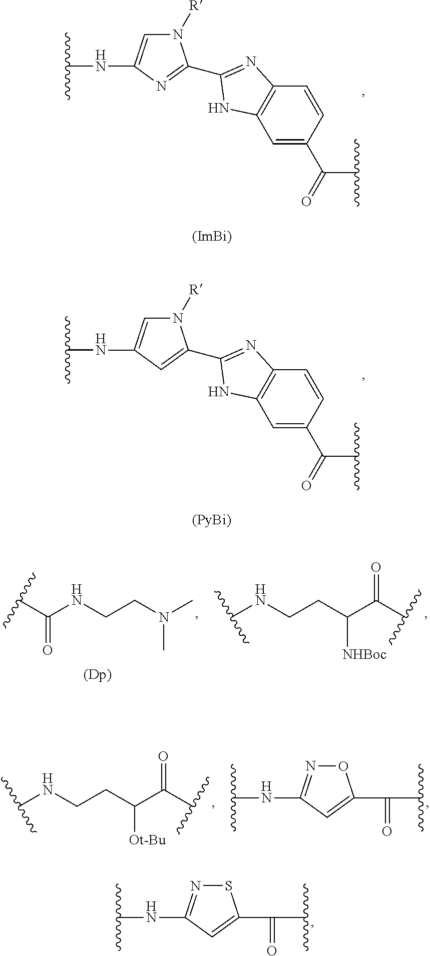

—NH-benzopyrazinylene-C(O)—, —NH-phenylene-C(O)—, —NH-pyridinylene-C(O)—, —NH-piperidinylene-C(O)—, —NH-pyrimidinylene-C(O)—, —NH-anthracenylene-C(O)—, —NH-quinolinylene-C(O)—, and

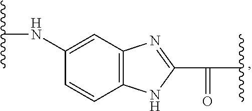

wherein each R′ is independently hydrogen, optionally substituted C₁-C₂₀ alkyl, optionally substituted C₁-C₂₀ heteroalkyl, optionally substituted C₁-C₂₀ haloalkyl, or optionally substituted C₁-C₂₀ alkylamino; and Z is H, NH₂, C₁-C₆ alkyl, C₁-C₆ haloalkyl, or C₁-C₆ alkyl-NH₂.

In some embodiments, the first terminus is a DNA-binding moiety that comprises a structure of Formula (A-1):

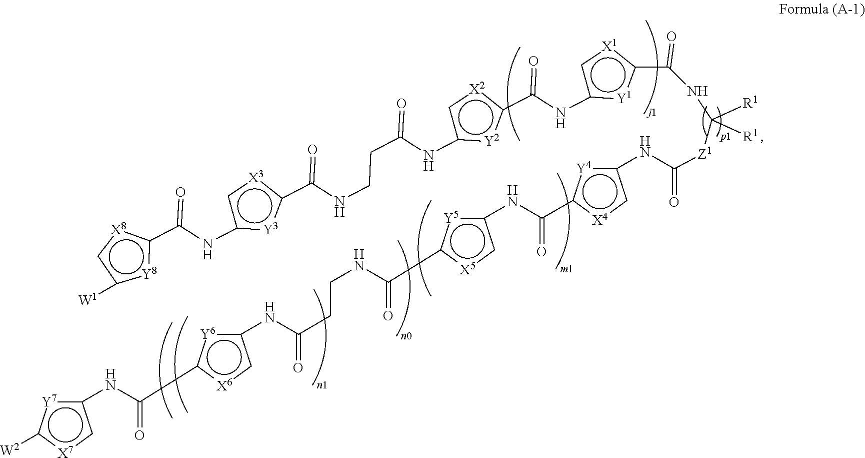

or a pharmaceutically acceptable salt thereof, wherein:
Z¹ is absent, —O— or —NH—;
each X¹, X², X³, X⁴, X⁵, X⁶, X⁷, and X⁸ is independently O, S, or NR²;
each Y¹, Y², Y³, Y⁴, Y⁵, Y⁶, Y⁷, and Y⁸ is independently CH or N;
W¹ is deuterium, hydrogen, optionally substituted C₁-C₆ alkyl, (azaneylidene)methanediamine, (azaneylidene)-N,N,N′,N′-tetramethylmethanediamine, —C(O)—NR^{1A}R^{1B}, —NR^{1A}—C(O)—NR^{1A}R^{1B}, —Z_{B}—P(O)(OR^{1A})₂, —Z_{B}—(CH₂)ₚ₃—PO(OR^{1A})₂, —Z_{B}—(CH₂)ₚ₃—O—P(O)(OR^{1A})₂, wherein
   Z_{B} is —N— or —O—;
   p₃ is an integer from 1-10;
W² is an optionally substituted C₁-C₆ alkyl or —C(O)—NR^{1A}R^{1B};
each R¹ is independently hydrogen, deuterium, halogen, amino, cyano, optionally C₁-C₂₀ alkyl, optionally substituted C₁-C₂₀ heteroalkyl, or —NC(O)R^{1A}; or
two R¹ on the same or on adjacent atoms combine together with the atom(s) to which they are attached to form an optionally substituted 3 to 6-membered carbocyclic ring or optionally substituted 3 to 6-membered heterocyclic ring;
each R² is independently hydrogen, deuterium, optionally substituted C₁-C₂₀ alkyl, optionally substituted C₁-C₂₀ heteroalkyl, optionally substituted C₁-C₂₀ haloalkyl, or optionally substituted C₁-C₂₀ alkylamino;
each R^{1A} is independently hydrogen, deuterium, or optionally substituted C₁-C₂₀ alkyl;
each R^{1B} is independently hydrogen, deuterium, optionally substituted C₁-C₂₀ alkyl, optionally substituted C₂-C₁₀ heteroalkyl, optionally substituted 5-membered heteroaryl, or (AA)ₚ₂, wherein
   each AA is an amino acid;
   p² is an integer from 1-10;
j₁ is 0 or 1;
n₀ is 1 or 0;
m₁ and n₁ are each independently an integer from 0-3;
p₁ is 2 or 3, provided that when Z¹ is —O— or —NH— then p₁ is 2, and when Z¹ is absent then p₁ is 3; and
wherein one of W¹ or W² is attached to the oligomeric backbone.

In some embodiments, n₀ is 1.

In some embodiments, the first terminus is a DNA-binding moiety that comprises a structure of Formula (A-2):

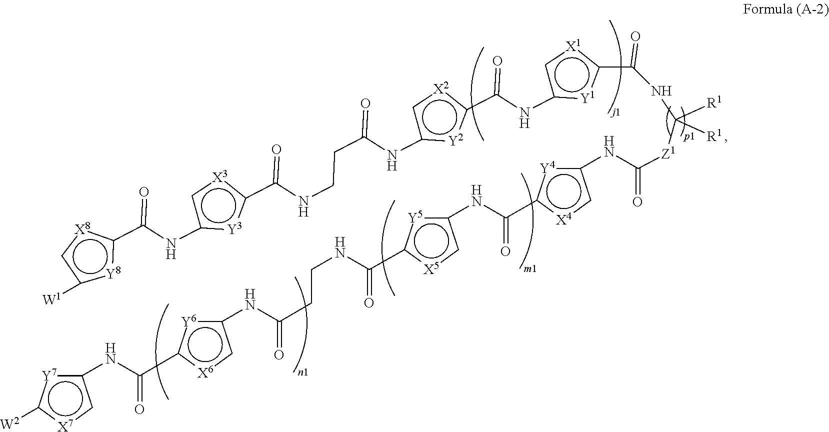

or a pharmaceutically acceptable salt thereof, wherein:
Z¹ is absent, —O—, or —NH—;
each X¹, X², X³, X⁴, X⁵, X⁶, X⁷, and X⁸ is independently O, S, or NR²;
each Y¹, Y², Y³, Y⁴, Y⁵, Y⁶, Y⁷, and Y⁸ is independently CH or N;
W¹ is deuterium, hydrogen, optionally substituted C₁-C₆ alkyl, (azaneylidene)methanediamine, (azaneylidene)-N,N,N′,N′-tetramethylmethanediamine, —C(O)—NR^{1A}R^{1B}, —NR^{1A}—C(O)—NR^{1A}R^{1B}, —Z_{B}—P(O)(OR^{1A})₂, —Z_{B}—(CH₂)ₚ₃—P(O)(OR^{1A})₂, —Z_{B}—(CH₂)ₚ₃—O—P(O)(OR^{1A})₂, wherein
   Z_{B} is —N— or —O—;
   p₃ is an integer from 1-10;
W² is an optionally substituted C₁-C₆ alkyl or —C(O)—NR^{1A}R^{1B};
each R¹ is independently hydrogen, deuterium, halogen, amino, cyano, optionally C₁-C₂₀ alkyl, optionally substituted C₁-C₂₀ heteroalkyl, or —NC(O)R^{1A}; or
two R¹ on the same or on adjacent atoms combine together with the atom(s) to which they are attached to form an optionally substituted 3 to 6-membered carbocyclic ring or optionally substituted 3 to 6-membered heterocyclic ring;
each R² is independently hydrogen, deuterium, optionally substituted C₁-C₂₀ alkyl, optionally substituted C₁-C₂₀ heteroalkyl, optionally substituted C₁-C₂₀ haloalkyl, or optionally substituted C₁-C₂₀ alkylamino;
each R^{1A} is independently hydrogen, deuterium, or optionally substituted C₁-C₂₀ alkyl;
each R^{1B} is independently hydrogen, deuterium, optionally substituted C₁-C₂₀ alkyl, optionally substituted C₂-C₁₀ heteroalkyl, optionally substituted 5-membered heteroaryl, or (AA)ₚ₂, wherein
   each AA is an amino acid;
   p₂ is an integer from 1-10;
j₁ is 0 or 1;
m₁ and n₁ are each independently an integer from 0-3;
p₁ is 2 or 3, provided that when Z¹ is —O— or —NH— then p₁ is 2, and when Z¹ is absent then p₁ is 3; and
   wherein one of W¹ or W² is attached to the oligomeric backbone.

In some embodiments, n₀ is 0.

In some embodiments, the DNA-binding moiety comprises the structure of Formula (A-3), or a pharmaceutically acceptable salt thereof:

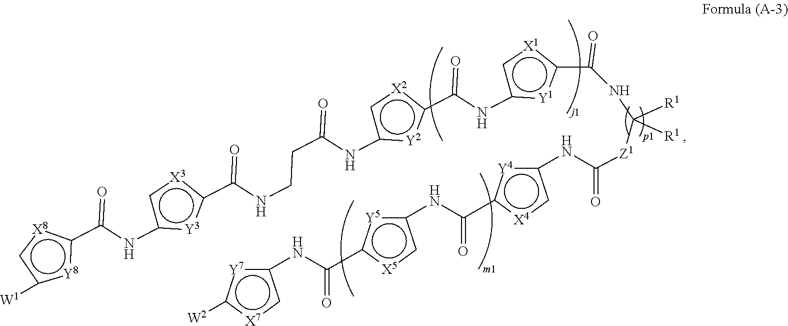

or a pharmaceutically acceptable salt thereof, wherein:
Z¹ is absent, —O—, or —NH—;
each X¹, X², X³, X⁴, X⁵, X⁶, X⁷, and X⁸ is independently O, S, or NR²;
each Y¹, Y, Y³, Y⁴, Y⁵, Y⁶, Y⁷, and Y⁸ is independently CH or N;
W¹ is deuterium, hydrogen, optionally substituted C₁-C₆ alkyl, (azaneylidene)methanediamine, (azaneylidene)-N,N,N′,N′-tetramethylmethanediamine, —C(O)—NR^{1A}R^{1B}, —NR^{1A}—C(O)—NR^{1A}R^{1B}, —Z_{B}—P(O)(OR^{1A})₂, —Z_{B}—(CH₂)ₚ₃—P(O)(OR^{1A})₂, —Z_{B}—(CH₂)ₚ₃—O—P(O)(OR^{1A})₂, wherein
   Z_{B} is —N— or —O—;
   p₃ is an integer from 1-10;
W² is an optionally substituted C₁-C₆ alkyl or —C(O)—NR^{1A}R^{1B};
each R¹ is independently hydrogen, deuterium, halogen, amino, cyano, optionally C₁-C₂₀ alkyl, optionally substituted C₁-C₂₀ heteroalkyl, or —NC(O)R^{1A}; or
two R¹ on the same or on adjacent atoms combine together with the atom(s) to which they are attached to form an optionally substituted 3 to 6-membered carbocyclic ring or optionally substituted 3 to 6-membered heterocyclic ring;
each R² is independently hydrogen, deuterium, optionally substituted C₁-C₂₀ alkyl, optionally substituted C₁-C₂₀ heteroalkyl, optionally substituted C₁-C₂₀ haloalkyl, or optionally substituted C₁-C₂₀ alkylamino;
each R^{1A} is independently hydrogen, deuterium, or optionally substituted C₁-C₂₀ alkyl;
each R^{1B} is independently hydrogen, deuterium, optionally substituted 5-membered heteroaryl, optionally substituted C₁-C₂₀ alkyl, optionally substituted C₂-C₁₀ heteroalkyl, or (AA)ₚ₂, wherein
   each AA is an amino acid;
   p₂ is an integer from 1-10;
j₁ is 0 or 1;
m₁ is an integer from 0-3;
p₁ is 2 or 3, provided that when Z¹ is —O— or —NH—, then p₁ is 2; and when Z₁ is absent then p₁ is 3; and
   wherein one of W¹ or W² is attached to the oligomeric backbone.

In some embodiments, Z¹ is absent. In some embodiments, Z¹ is —O— or —NH—.

In some embodiments, W² is —C(O)NR^{1A}R^{1B}, wherein W² is attached to the oligomeric backbone. In some embodiments, R^{1A} is hydrogen and R^{1B} is AA, wherein AA is beta alanine. In some embodiments, W² is —C(O)NH-(beta-alanine)-. In some embodiments, W² is —C(O)NR^{1A}R^{1B}, wherein R^{1A} is hydrogen and R^{1B} is alkyl optionally substituted with an oxo (═O). In some embodiments, W² is —C(O)NH(CH₂)₂C(O)—. In some embodiments, W² is —C(O)NH—.

In some embodiments, the DNA-binding moiety is connected to the oligomeric backbone through W². In some embodiments, the oligomeric backbone is a linker moiety. In some embodiments, the DNA-binding moiety is not connected to the oligomeric backbone through W². In some embodiments, W² is —C(O)NH(CH₂)₂C(O)—**, wherein the linker moiety is attached at **. In some embodiments, W² is —C(O)O(CH₂)₂C(O)—**, wherein the linker moiety is attached at **. In some embodiments, W² is —C(O)—NH—**, wherein the linker moiety is attached at **. In some embodiments, W² is —C(O)OH—**, wherein the linker moiety is attached at **. In some embodiments, W² is —C(O)—**, wherein the linker moiety is attached at **.

In some embodiments, the DNA-binding moiety comprises the structure of Formula (A-4), or a pharmaceutically acceptable salt thereof:

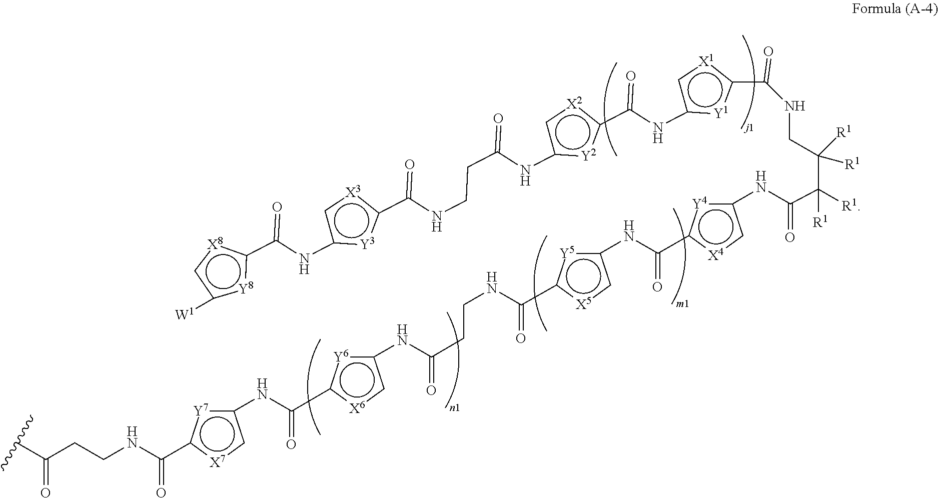

In some embodiments, each R¹ is independently halogen, amino, cyano, optionally C₁-C₂₀ alkyl, or optionally substituted C₁-C₂₀ heteroalkyl. In some embodiments, R¹ is C₁-C₂₀ alkyl or C₁-C₂₀ heteroalkyl. In some embodiments, each R¹ is independently —NHC(O)R^{1A}, wherein R^{1A} is alkyl, aryl, or heteroaryl. In some embodiments, each R¹ is independently —NH₂, —NHCH₃, or —NHC(O)CH(CH₃)₃. In some embodiments, each R¹ is hydrogen.

In some embodiments, two R¹ on the same or on adjacent atoms combine together with the atom(s) to which they are attached to form an optionally substituted 3-6 membered carbocyclic ring or 3-6 membered heterocyclic ring. In some embodiments, two R¹ on the same carbon atom combine together to form an optionally substituted 3-6 membered carbocyclic ring or 3-6 membered heterocyclic ring. In some embodiments, the two R¹ on the same carbon atom combine together to form an optionally substituted 3-6 membered carbocyclic ring. In some embodiments, the carbocyclic ring is a cyclopropyl, cyclobutyl, cyclopentyl, or cyclohexyl ring. In some embodiments, two R¹ on the same carbon atom combine together to form an optionally substituted 3-6 membered heterocyclic ring, optionally containing 1-2 heteroatoms selected from N, O, or S. In some embodiments, the heterocyclic ring is an oxetane, tetrahydrofuran, or tetrahydro-2H-pyran.

In some embodiments, two R¹ on adjacent atoms combine together with the atom(s) to which they are attached to form an optionally substituted 3-6 membered carbocyclic ring or 3-6 membered heterocyclic ring. In some embodiments, two R¹ on adjacent atoms combine together with the atom(s) to which they are attached to form an optionally substituted 3-6 membered carbocyclic ring. In some embodiments, two R¹ on adjacent atoms combine together with the atom(s) to which they are attached to form an optionally substituted 3-6 membered heterocyclic ring. In some embodiments, the cyclization occurs between the a and the p carbon atoms or between the p and the 6 carbon atoms.

In some embodiments, the DNA-binding moiety comprises the structure of Formula (A-5):

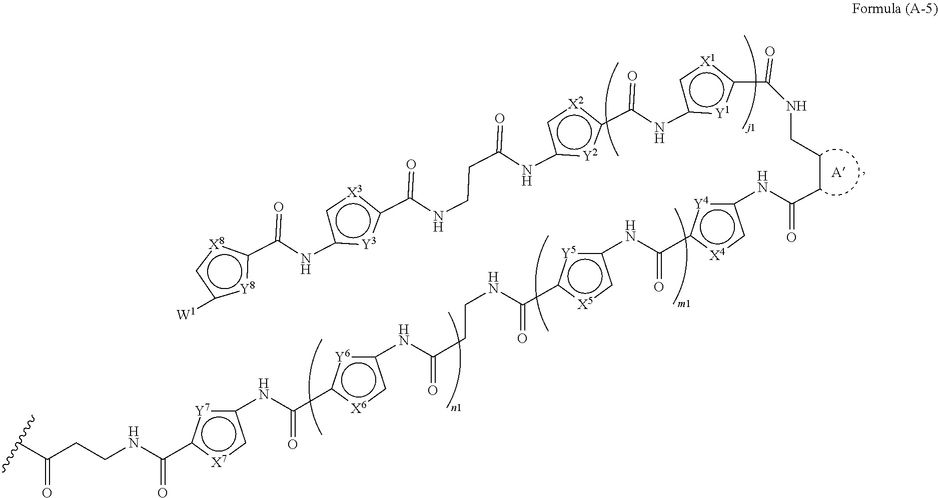

or a pharmaceutically acceptable salt thereof, wherein:
Ring A′ is an optionally substituted C₃-C₆ carbocyclic ring or optionally substituted 3 to 6-membered heterocyclic ring.

In some embodiments, the DNA-binding moiety comprises the structure of Formula (A-6), or a pharmaceutically acceptable salt thereof:

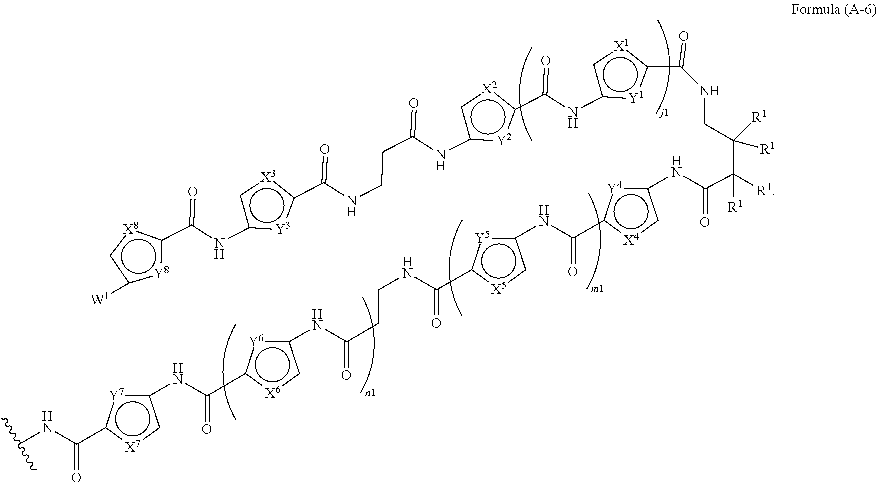

In some embodiments, the DNA-binding moiety comprises the structure of Formula (A-7), or a pharmaceutically acceptable salt thereof:

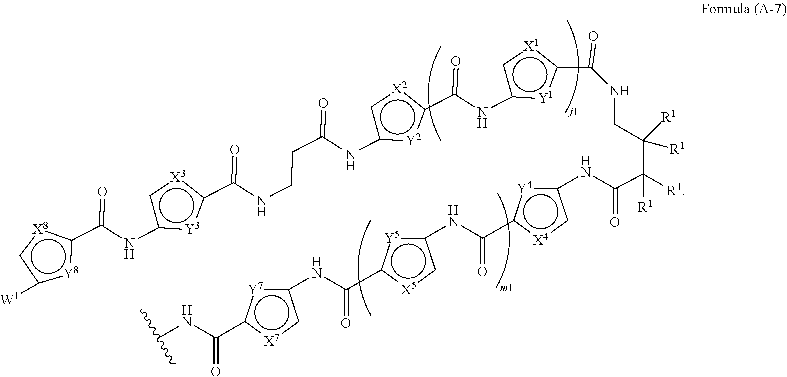

In some embodiments, Y², Y⁴, and Y⁷ are each independently N; and Y¹ and Y³ are each independently CH.

In some embodiments, Y⁶ is CH. In some embodiments, Y⁶ is N.

In some embodiments, X¹, X², X³, X⁴, X⁵, X⁶, and X⁷ are each independently —NR².

In some embodiments, the DNA-binding moiety comprises the structure of Formula (A-8):

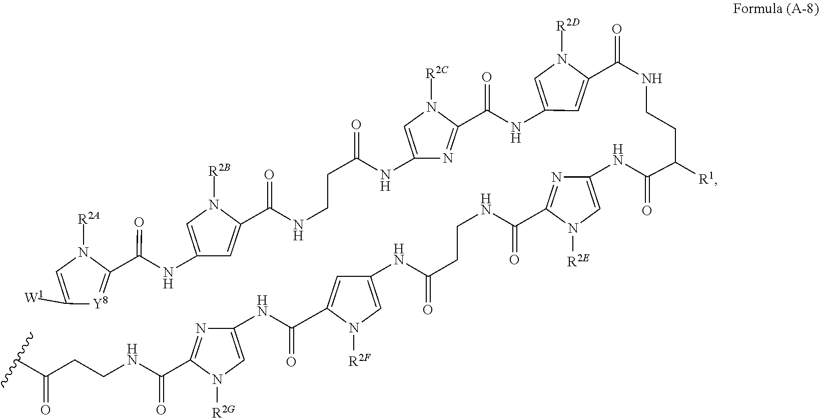

or a pharmaceutically acceptable salt thereof, wherein:
Y⁸ is CH or N; and
R^{2A}, R^{2B}, R^{2c}, R^{2D}, R^{2E}, R^{2F}, and R^{2G} are each independently hydrogen, deuterium, optionally substituted C₁-C₂₀ alkyl, optionally substituted C₁-C₂₀ heteroalkyl, optionally substituted C₁-C₂₀ haloalkyl, or optionally substituted C₁-C₂₀ alkylamino.

In some embodiments, the DNA-binding moiety comprises the structure of Formula (A-9):

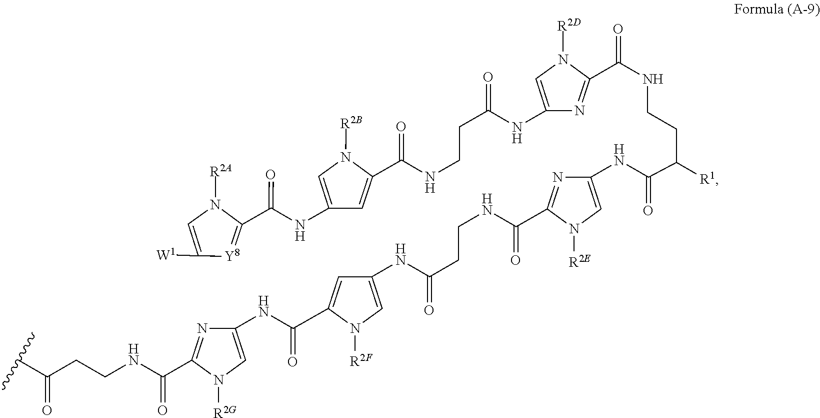

or a pharmaceutically acceptable salt thereof, wherein:
Y⁸ is CH or N; and
R^{2A}, R^{2B}, R^{2D}, R^{2E}, R^{2F}, and R^{2G} are each independently hydrogen, deuterium, optionally substituted C₁-C₂₀ alkyl, optionally substituted C₁-C₂₀ heteroalkyl, optionally substituted C₁-C₂₀ haloalkyl, or optionally substituted C₁-C₂₀ alkylamino.

In some embodiments, the DNA-binding moiety comprises the structure of Formula (A-10):

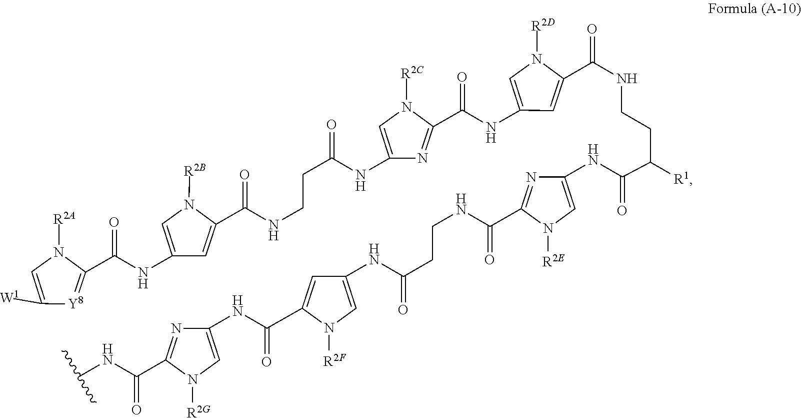

or a pharmaceutically acceptable salt thereof, wherein:
Y⁸ is CH or N; and
R^{2A}, R^{2B}, R^{2C}, R^{2D}, R^{2E}, R^{2F}, and R^{2G} are each independently hydrogen, deuterium, optionally substituted C₁-C₂₀ alkyl, optionally substituted C₁-C₂₀ heteroalkyl, optionally substituted C₁-C₂₀ haloalkyl, or optionally substituted C₁-C₂₀ alkylamino.

In some embodiments, Y⁸ is N. In some embodiments, Y⁸ is CH.

In some embodiments, R^{2A}, R^{2B}, R^{2c}, R^{2D}, R^{2E}, R^{2F}, and R^{2G} are each independently hydrogen, deuterium, optionally substituted C₁-C₂₀ alkyl, optionally substituted C₁-C₂₀ haloalkyl, or optionally substituted C₁-C₂₀ heteroalkyl.

In some embodiments, R^{2A}, R^{2B}, R^{2c}, R^{2D}, R^{2E}, R^{2F}, and R^{2G} are each independently an optionally substituted C₁-C₂₀ alkyl. In some embodiments, R^{2A}, R^{2B}, R^{2c}, R^{2D}, R^{2E}, R^{2F}, and R^{2G} are each independently a straight chain or branched C₁-C₂₀ alkyl. In some embodiments, R^{2A}, R^{2B}, R^{2c}, R^{2D}, R^{2E}, R^{2F}, and R^{2G} are each independently an optionally substituted methyl, ethyl, isopropyl, n-butyl, isobutyl, sec-butyl, or tert-butyl. In some embodiments, R^{2A}, R^{2B}, R^{2c}, R^{2D}, R^{2E}, R^{2F}, and R^{2G} are each independently methyl, ethyl, or tert-butyl.

In some embodiments, the DNA-binding moiety comprises the structure of Formula (A-11), or a salt thereof:

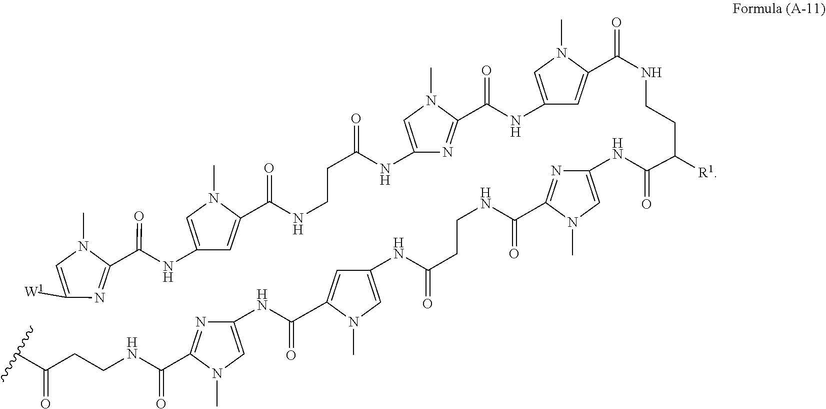

In some embodiments, the DNA-binding moiety comprises the structure of Formula (A-12), or a salt thereof:

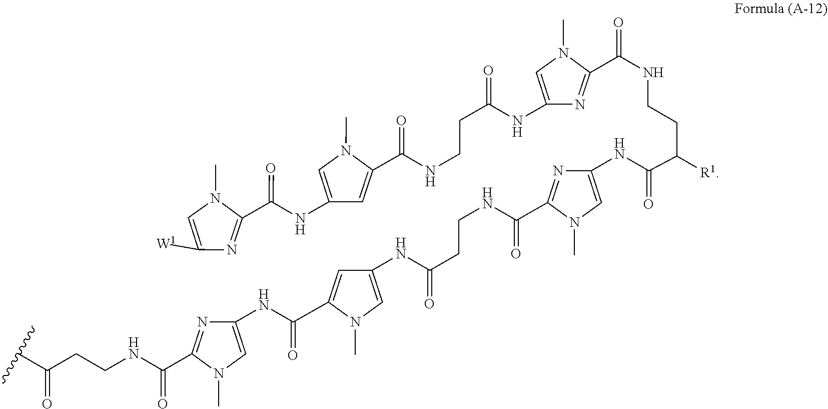

In some embodiments, the DNA-binding moiety comprises the structure of Formula (A-13), or a salt thereof:

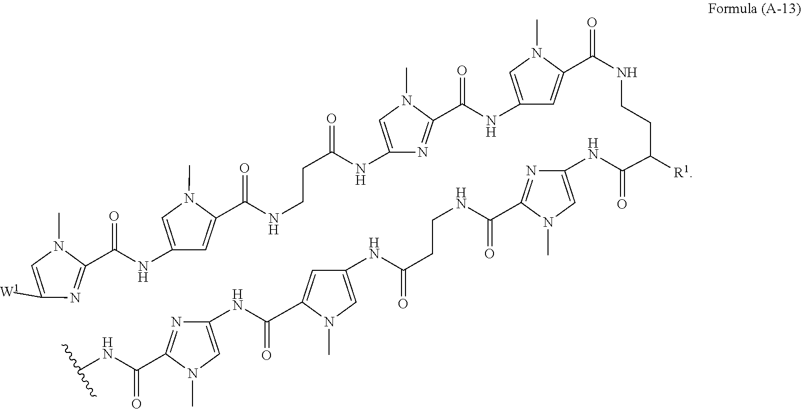

In some embodiments, W¹ is —C(O)—NR^{1A}R^{1B} or —NR^{1A}—C(O)—NR^{1A}R^{1B}.

In some embodiments, W¹ is hydrogen.

In some embodiments, W¹ is —Z_{B}—P(O)(OR^{1A})₂, —Z_{B}—(CH₂)ₚ₃—P(O)(OR^{1A})₂. —Z_{B}—(CH₂)ₚ₃—O—P(O)₂(OR^{1A})₂, wherein Z_{B} is O or N, and p₃ is an integer from 1-10.

In some embodiments, W¹ is (azaneylidene)methanediamine or (azaneylidene)-N,N,N′,N′-tetramethylmethanediamine. In some embodiments, W¹ is

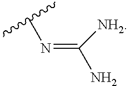 [SMILES: *N=C(N)N]

In some embodiments, W¹ is

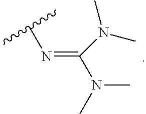 [SMILES: *N=C(N(C)C)N(C)C]

In some embodiments, the DNA-binding moiety is connected to the oligomeric backbone through W¹. In some embodiments, the oligomeric backbone is a linker moiety. In some embodiments, the DNA-binding moiety is not connected to the oligomeric backbone through W¹.

In some embodiments, m₁ is 0. In some embodiments, m₁ is 1. In some embodiments, m₁ is 2. In some embodiments, m₁ is 3.

In some embodiments, p₁ is 2. In some embodiments, p₁ is 3.

In some embodiments, m₁ is 0 or 1 and p₁ is 2.

In some embodiments, n₁ is 0. In some embodiments, n₁ is 1. In some embodiments, n₁ is 2. In some embodiments, n₁ is 3.

In some embodiments, j₁ is 0. In some embodiments, j₁ is 1.

The binding affinity between the polyamide and the target gene can be adjusted based on the composition of the polyamide. In some embodiments, the polyamide is capable of binding the DNA with an affinity of less than about 600 nM, about 500 nM, about 400 nM, about 300 nM, about 250 nM, about 200 nM, about 150 nM, about 100 nM, or about 50 nM. In some embodiments, the polyamide is capable of binding the DNA with an affinity of less than about 300 nM. In some embodiments, the polyamide is capable of binding the DNA with an affinity of less than about 200 nM. In some embodiments, the polyamide is capable of binding the DNA with an affinity of greater than about 200 nM, about 150 nM, about 100 nM, about 50 nM, about 10 nM, or about 1 nM. In some embodiments, the polyamide is capable of binding the DNA with an affinity in the range of about 1-600 nM, 10-500 nM, 20-500 nM, 50-400 nM, or 100-300 nM.

In some embodiments, the first terminus is capable of binding the DNA with an affinity of less than 500 nM.

The binding affinity between the polyamide and the target DNA can be determined using a quantitative footprint titration experiment. The experiment involves measuring the dissociation constant K_{d} of the polyamide for the target sequence at either 24° C. or 37° C., and using either standard polyamide assay solution conditions or approximate intracellular solution conditions.

The binding affinity between the regulatory protein and the ligand on the second terminus can be determined using an assay suitable for the specific protein. The experiment involves measuring the dissociation constant K_{d} of the ligand for the protein and using either standard protein assay solution conditions or approximate intracellular solution conditions.

The first terminus DNA binding moiety in the molecules described herein has a high binding affinity to a sequence having multiple repeats of CAG and binds to the target nucleotide repeats preferentially over other nucleotide repeats or other nucleotide sequences. In some embodiments, the first terminus has a higher binding affinity to a sequence having multiple repeats of CAG than to a sequence having repeats of CGG. In some embodiments, the first terminus has a higher binding affinity to a sequence having multiple repeats of CAG than to a sequence having repeats of CCG. In some embodiments, the first terminus has a higher binding affinity to a sequence having multiple repeats of CAG than to a sequence having repeats of CCTG. In some embodiments, the first terminus has a higher binding affinity to a sequence having multiple repeats of CAG than to a sequence having repeats of TGGAA. In some embodiments, the first terminus has a higher binding affinity to a sequence having multiple repeats of CAG than to a sequence having repeats of GGGGCC. In some embodiments, the first terminus has a higher binding affinity to a sequence having multiple repeats of CAGCTG than to a sequence having repeats of GAA.

Due to the preferential binding between the first terminus and the target nucleotide repeat, the transcription modulation molecules described herein become localized around regions having multiple repeats of CAG. In some embodiments, the local concentration of the first terminus of the molecules described herein is higher near a sequence having multiple repeats of CAG than near a sequence having repeats of CGG. In some embodiments, the local concentration of the first terminus of the molecules described herein is higher near a sequence having multiple repeats of CAG than near a sequence having repeats of CCG. In some embodiments, the local concentration of the first terminus of the molecules described herein is higher near a sequence having multiple repeats of CAG than near a sequence having repeats of CCTG. In some embodiments, the local concentration of the first terminus or the molecules described herein is higher near a sequence having multiple repeats of CAG than near a sequence having repeats of TGGAA. In some embodiments, the local concentration of the first terminus of the molecules described herein is higher near a sequence having multiple repeats of CAG than near a sequence having repeats of GGGGCC. In some embodiments, the local concentration of the first terminus of the molecules described herein is higher near a sequence having multiple repeats of CAG than near a sequence having repeats of GAA.

The first terminus DNA binding moiety in the molecules described herein is localized to a sequence having multiple repeats of CAG and binds to the target nucleotide repeats preferentially over other nucleotide repeats. In some embodiments, the sequence has at least 2, 3, 4, 5, 8, 10, 12, 15, 20, 25, 30, 40, 50, 100, 200, 300, 400, or 500 repeats of CAG. In some embodiments, the sequence comprises at least 1000 nucleotide repeats of CAG. In certain embodiments, the sequence comprises at least 500 nucleotide repeats of CAG. In certain embodiments, the sequence comprises at least 200 nucleotide repeats of CAG. In some embodiments, the sequence comprises at least 100 nucleotide repeats of CAG. In some embodiments, the sequence comprises at least 50 nucleotide repeats of CAG. In some embodiments, the sequence comprises at least 20 nucleotide repeats of CAG.

The polyamide composed of a pre-selected combination of subunits can selectively bind to the DNA in the minor groove. In their hairpin structure, antiparallel side-by-side pairings of two aromatic amino acids bind to DNA sequences, with a polyamide ring packed specifically against each DNA base. N-Methylpyrrole (Py) favors T, A, and C bases, excluding G; N-methylimidazole (Im) is a G-reader; and 3-hydroxyl-N-methylpyrrol (Hp) is specific for thymine base. The nucleotide base pairs can be recognized using different pairings of the amino acid subunits using the pairing principle shown in Table 1A and 1B below. For example, an Im/Py pairing reads GC by symmetry, a Py/Im pairing reads C G, an Hp/Py pairing can distinguish T A from AT, G C, and C G, and a Py/Py pairing nonspecifically discriminates both AT and T A from GC and C G.

In some embodiments, the first terminus comprises Im corresponding to the nucleotide G; Im or Nt corresponding to the nucleotide pair G; Py corresponding to the nucleotide C, wherein Im is N-alkyl imidazole, Py is N-alkyl pyrrole, Hp is 3-hydroxy N-methyl pyrrole, and β-alanine. In some embodiments, the first terminus comprises Im/Py to correspond to the nucleotide pair G/C, Py/Im to correspond to the nucleotide pair C/G, and wherein Im is N-alkyl imidazole (e.g., N-methyl imidazole), Py is N-alkyl pyrrole (e.g., N-methyl pyrrole), and Hp is 3-hydroxy N-methyl pyrrole.

TABLE 1A
Base pairing for single amino acid subunit (Favored (+), disfavored (−)).

| Subunit | G | C | A | T |
|---|---|---|---|---|
| Py | − | + | + | + |
| Im | + | − | − | |
| 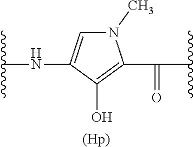 | − | − | − | + |
| 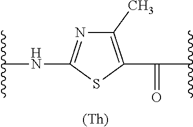 | − | − | + | + |
| 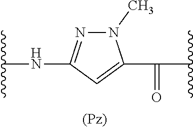 | − | − | + | + |
| 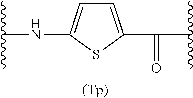 | − | − | + | + |
| 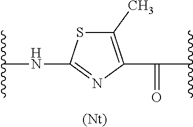 | + | − | − | − |
| 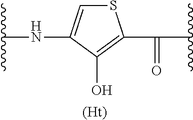 | − | − | − | + |
| 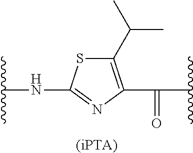 | + | − | − | − |
| 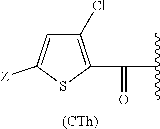 | − | − | − | + |
| 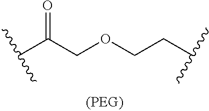 [SMILES: *CCOCC(*)=O] | − | + | + | + |
| 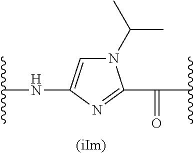 | + | − | − | − |
| 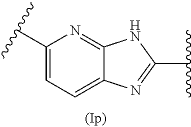 [SMILES: *c1ccc2nc(*)[nH]c2n1] | + | − | − | − |
| 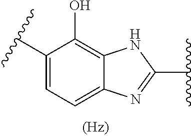 [SMILES: *c1nc2ccc(*)c(O)c2[nH]1] | − | − | − | + |
| 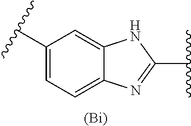 | − | − | − | + |
| 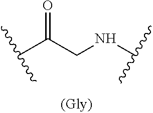 [SMILES: *NCC(*)=O] | − | − | − | − |
| 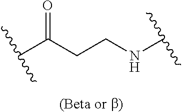 [SMILES: *NCCC(*)=O] | − | − | + | + |
| 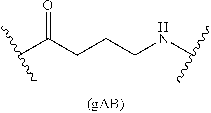 [SMILES: *NCCCC(*)=O] | − | − | + (as a part of the turn) | + (as a part of the turn) |
| 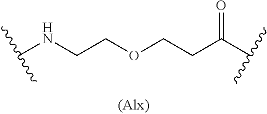 [SMILES: *NCCOCCC(*)=O] | − | + | − | − |
| 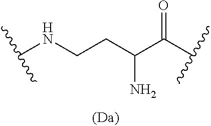 [SMILES: *NCCC(N)C(*)=O] | − | − | + | + |
| 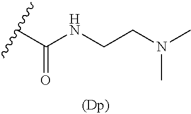 [SMILES: *C(=O)NCCN(C)C] | − | − | + | + |
| 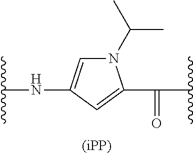 | − | − | + | + |
| 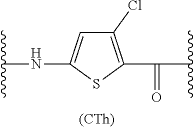 [SMILES: *Nc1cc(Cl)c(C(*)=O)s1 ; "(CTh)"] | + | + | − | − |
| 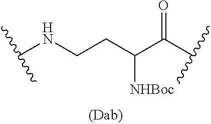 [SMILES: *NCCC(NC(=O)OC(C)(C)C)C(*)=O] | − | − | + | + |
| 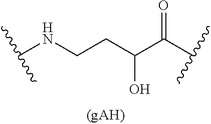 [SMILES: *NCCC(O)C(*)=O] | − | − | + | + |

| | |
|---|---|
| 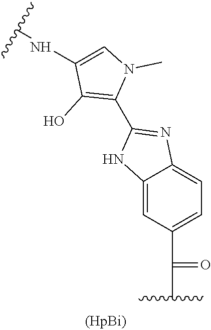 [SMILES: *Nc1cn(C)c(-c2nc3ccc(C(*)=O)cc3[nH]2)c1O] | WW* (bind to two nucleotides with same selectivity as Hp-Py) |
| 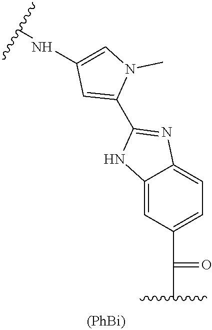 [SMILES: *Nc1cc(-c2nc3ccc(C(*)=O)cc3[nH]2)n(C)c1] | WW* (bind to two nucleotides with same selectivity as Py-Py) |
| 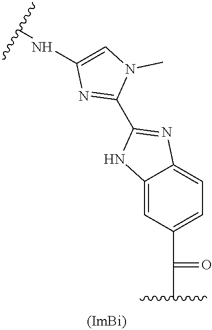 [SMILES: *Nc1cn(C)c(-c2nc3ccc(C(*)=O)cc3[nH]2)n1] | GW* (bind to two nucleotides with same selectivity as Im-Py) |

*The subunit HpBi, ImBi, and PyBi function as a conjugate of two monomer subunits and bind to two nucleotides. The binding property of HpBi, ImBi, and PyBi corresponds to Hp-Py, Im-Py, and Py-Py respectively.

TABLE 1B
Representative base pairing for hairpin polyamide.

| | G•C | C•G | T•A | A•T |
|---|---|---|---|---|
| Im/ß | + | − | − | − |
| ß/Im | − | + | − | − |
| Py/ß | − | − | + | + |
| ß/Py | − | − | + | + |
| ß/ß | − | − | + | + |
| Py/Py | − | − | + | + |
| Im/Im | − | − | − | − |
| Im/Py | + | − | − | − |
| Py/Im | − | + | − | − |
| Th/Py | − | − | + | − |
| Py/Th | − | − | − | + |
| Th/Im | + | − | − | − |
| Im/Th | − | + | − | − |
| ß/Th | − | − | + | − |
| Th/ß | − | − | − | + |
| Hp/Py, | − | − | + | − |
| Py/Hp, | − | − | − | + |
| Hp/Im | + | − | − | − |
| Im/Hp | − | + | − | − |
| Tn/Py | − | − | + | + |
| Py/Tn, | − | − | + | + |
| Ht/Py, | − | − | + | + |
| Py/Ht, | − | − | + | + |
| Bi/Py, | − | − | + | + |
| Py/Bi, | − | − | + | + |
| ß/Bi | − | − | + | + |
| Bi/ß | − | − | + | + |
| Bi/Im, | − | + | − | − |
| Im/Bi, | + | − | − | − |
| Tp/Py, | − | − | + | + |
| Py/Tp, | − | − | + | + |
| ß/Tp | − | − | + | + |
| Tp/ß | − | − | + | + |
| Tp/Im, | − | + | − | − |
| Im/Tp | + | − | − | − |
| Tp/Tp | − | − | + | + |
| Tp/Tn | − | − | + | + |
| Tn/Tp | − | − | + | + |
| Hz/Py, | − | − | + | − |
| Py/Hz, | − | − | − | + |
| Ip/Py | + | − | − | − |
| Py/Ip, | − | + | − | − |
| Bi/Hz, | − | − | + | + |
| Hz/Bi, | − | − | + | + |
| Bi/Bi | − | + | + | + |
| Th/Py, | − | − | + | + |
| Py/Th | − | − | + | + |
| Im/gAB | + | − | − | − |
| gAB/Im | − | + | − | − |
| Py/gAB | + | − | − | − |
| gAB/Py | − | + | − | − |
| gAB/β | − | − | + | + |
| ß/gAB | − | − | + | + |
| Im/Dp | + | − | − | − |
| Dp/Im | − | + | − | − |
| Py/Dp | − | − | + | + |
| Dp/Py | − | − | + | + |
| Dp/ß | − | − | + | + |

Each of HpBi, ImBi, and PyBi can bind to two nucleotides and have binding properties corresponding to Hp-Py, Im-Py, and Py-Py respectively. HpBi, ImBi, and PyBi can be paired with two monomer subunits or with themselves in a hairpin structure to bind to two nucleotide pairs.

The monomer subunits of the polyamide can be strung together based on the pairing principles shown in Table 1A and Table 1B. The monomer subunits of the polyamide can be strung together based on the pairing principles shown in Table 1C.

Table 1C shows an example of the monomer subunits that can bind to the specific nucleotide. The first terminus can include a polyamide described as having several monomer subunits strung together, with a monomer subunit selected from each row. For example, the polyamide can include Py-Py-Im that binds to CAG, where Py is selected from the C column, Py is selected from the A column, and Im selected from the first G column. The polyamide can be any combinations of the subunits of CAGCAG, with a subunit selected from each column in Table 1C, wherein the subunits are strung together following the CAG binding order.

In addition, the polyamide can also include a partial or multiple sets of the five subunits, such as 1.5, 2, 2.5, 3, 3.5, or 4 sets of the three subunits. The polyamide can include 3, 4, 5, 6, 7, 8, 9, 10, 12, 14, and 16 monomer subunits.

The polyamide can include monomer subunits that bind to 2, 3, 4, or 5 nucleotides of CAG. For example, the polyamide can bind to CA, CAG, AGC, CAGC, CAGCA, CAGCAG. The polyamide can include monomer subunits that bind to 3, 4, 5, 6, 7, 8, 9, or 10 nucleotides of CAG repeat.

The monomer subunit, when positioned as a terminal unit, does not have an amine, carbonyl, or a carboxylic acid group at the terminal. The carboxylic acid group in the terminal is replaced by a hydrogen. For example, Py, when used as a terminal unit, is understood to have the structure of

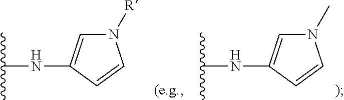

and Im, when positioned as a terminal unit, is understood to have the structure of

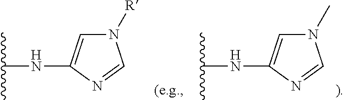

TABLE 1C
Examples of monomer subunits in a linear polyamide
that binds to CAG.

| Nucleotide | C | A | G |
|---|---|---|---|
| Subunit that | Py | Py | Im or ImT |
| selectively binds to | iIm or iImT | Th | iIm or iImT |
| nucleotide | PEG | Pz | PEG |
| | CTh | Tp | CTh |
| | Alx | PEG | Nt |
| | | ß | IPTA |
| | | iPP | Ip |
| | | Da | CTh |
| | | Dp | |
| | | Dab | |
| | | gAH | |

Recognition of a nucleotide repeat or DNA sequence by two antiparallel polyamide strands depends on a code of side-by-side aromatic amino acid pairs in the minor groove, usually oriented N to C with respect to the 5′ to 3′ direction of the DNA helix. Enhanced affinity and specificity of polyamide nucleotide binding is accomplished by covalently linking the antiparallel strands. The “hairpin motif” connects the N and C termini of the two strands with a gamma-aminobutyric acid unit (gamma-turn)) to form a folded linear chain. The “H-pin motif” connects the antiparallel strands across a central or near central ring/ring pairs by a short, flexible bridge.

Second Terminus—Regulatory Protein Binding Moiety

In some embodiments, the second terminus comprises a protein-binding moiety capable of binding to a regulatory molecule that modulates expression of a gene having the expanded nucleotide repeat.

In some embodiments, the second terminus comprises a bromodomain binding moiety.

In some embodiments, the second terminus comprises a moiety capable of binding to a bromodomain and extra terminal domain (BET) family member.

In some embodiments, the BET family member is BRD2, BRD3, BRD4, or BRDT. In some embodiments, the BET family member is BRD2. In some embodiments, the BET family member is BRD3. In some embodiments, the BET family member is BRD4. In some embodiments, the BET family member is BRD3. In some embodiments, the BET family member is BRDT.

In some embodiments, the bromodomain is CBP/p300, PCAF (P300/CBP-Associated Factor), CECR2 (cat eye syndrome chromosome region candidate 2), BRPF (bromodomain and PHD finger-containing protein), ATAD2/ATAD2B (chromatin remodeling proteins), TRIM24 (Tripartite motif-containing 24), BAZ2 (Bromodomain Adjacent to Zinc finger), or TAF1 (TBP associated factors).

In some embodiments, the bromodomain is CBP/p300.

In some embodiments, the bromodomain is PCAF (P300/CBP-Associated Factor).

In some embodiments, the bromodomain is CECR2 (cat eye syndrome chromosome region candidate 2).

In some embodiments, the bromodomain is BRPF (bromodomain and PHD finger-containing protein).

In some embodiments, the bromodomain is a ATAD2 or ATAD2B chromatin remodeling protein.

In some embodiments, the bromodomain is BAZ2 (Bromodomain Adjacent Zinc Finger.

In some embodiments, the bromodomain is TAF1 (TBP associated factor).

In some embodiments, the bromodomain is TRIM24 (tripartite motif-containing 24).

In some embodiments, the regulatory molecule modulates the rearrangement of histones.

In some embodiments, the regulatory molecule modulates the glycosylation, phosphorylation, alkylation, or acylation of histones.

In some embodiments, the regulatory molecule is a transcription factor.

In some embodiments, the regulatory molecule is an RNA polymerase.

In some embodiments, the regulatory molecule is a moiety that regulates the activity of RNA polymerase.

In some embodiments, the recruiting moiety binds to the regulatory molecule but does not inhibit the activity of the regulatory molecule. In some embodiments, the recruiting moiety binds to the regulatory molecule and inhibits the activity of the regulatory molecule. In some embodiments, the recruiting moiety binds to the regulatory molecule and increases the activity of the regulatory molecule.

In some embodiments, the recruiting moiety binds to the active site of the regulatory molecule. In certain embodiments, the recruiting moiety binds to a regulatory site of the regulatory molecule.

The binding affinity between the regulatory protein and the second terminus can be adjusted based on the composition of the molecule or type of protein. In some embodiments, the second terminus binds the regulatory molecule with an affinity of less than about 600 nM, about 500 nM, about 400 nM, about 300 nM, about 250 nM, about 200 nM, about 150 nM, about 100 nM, or about 50 nM. In some embodiments, the second terminus binds the regulatory molecule with an affinity of less than about 300 nM. In some embodiments, the second terminus binds the regulatory molecule with an affinity of less than about 200 nM.

In some embodiments, the second terminus comprises a diazine or diazepine ring, wherein the diazine or diazepine ring is fused with a C₆-C₁₀ aryl or a 5 to 10-membered heteroaryl ring comprising one or more heteroatoms selected from S, N and O. In some embodiments, the second terminus comprises an optionally substituted bicyclic or tricyclic structure.

In some embodiments, the second terminus has a triazolodiazepine structure. In some embodiments, the second terminus has a thiazolodiazepine structure.

In some embodiments, the second terminus comprises the structure of Formula (2-A):

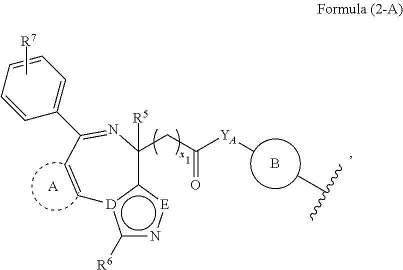

or a pharmaceutically acceptable salt thereof, wherein:
Ring A is an optionally substituted aryl or optionally substituted 5 to 6-membered heteroaryl;
Ring B is absent or an optionally substituted 6-membered monocyclic aryl or heteroaryl;
D is C or N;
E is O or N;
Y_{A} is —NH— or —O—;
R⁵ is hydrogen, deuterium, or C₁-C₆ alkyl; R⁶ is selected from hydrogen, optionally substituted C₁-C₆ alkyl, optionally substituted C₁-C₆ haloalkyl, and optionally substituted C₁-C₆ hydroxyalkyl;
R⁷ is selected from hydrogen, deuterium, halogen, —NO₂, —CN, optionally substituted aryl, optionally substituted C₁-C₂₀ alkyl, optionally substituted C₁-C₂₀ heteroalkyl, optionally substituted C₁-C₆ haloalkyl, and optionally substituted C₁-C₆ hydroxyalkyl;
or R⁷ is —NR^{7A}R^{7B}, wherein
   R^{7A} and R^{7B} are each independently hydrogen, deuterium, optionally substituted C₁-C₂₀ alkyl, or optionally substituted C₁-C₂₀ heteroalkyl; and
x₁ is an integer from 1-6.

In some embodiments, D is N and E is N. In some embodiments, D is C and E is 0.

In some embodiments, the second terminus comprises the structure of Formula (2-B):

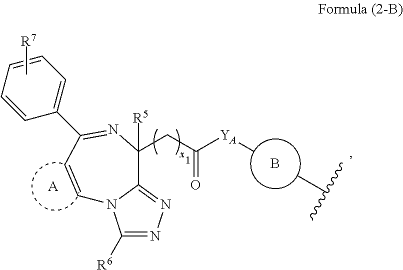

or a pharmaceutically acceptable salt thereof, wherein:
Ring A is an optionally substituted aryl or optionally substituted 5 to 6-membered heteroaryl;
Ring B is absent or an optionally substituted 6-membered monocyclic aryl or heteroaryl;
Y_{A} is —NH— or —O—;
R⁵ is hydrogen, deuterium, or C₁-C₆ alkyl;
R⁶ is selected from hydrogen, optionally substituted C₁-C₆ alkyl, optionally substituted C₁-C₆ haloalkyl, and optionally substituted C₁-C₆ hydroxyalkyl;
R⁷ is selected from hydrogen, deuterium, halogen, —NO₂, —CN, optionally substituted aryl, optionally substituted C₁-C₂₀ alkyl, optionally substituted C₁-C₂₀ heteroalkyl, optionally substituted C₁-C₆ haloalkyl, and optionally substituted C₁-C₆ hydroxyalkyl;
or R⁷ is —NR^{7A}R^{7B}, wherein
   R^{7A} and R^{7B} are each independently hydrogen, deuterium, optionally substituted C₁-C₂₀ alkyl, or optionally substituted C₁-C₂₀ heteroalkyl; and
x₁ is an integer from 1-6.

In some embodiments, Ring A is an optionally substituted aryl ring. In some embodiments, Ring A is an optionally substituted phenyl. In some embodiments, Ring A is an optionally substituted 5 membered heteroaryl. In some embodiments, Ring A is an optionally substituted oxazolyl. In some embodiments, Ring A is an optionally substituted furanyl. In some embodiments, Ring A is an optionally substituted thiophenyl.

In some embodiments, the second terminus comprising the structure of Formula (2-C):

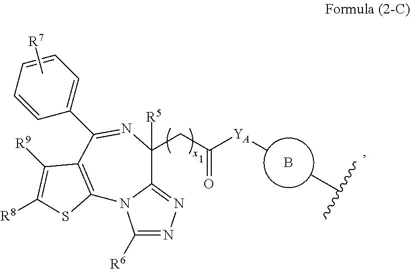

or a pharmaceutically acceptable salt thereof, wherein:
R⁸ and R⁹ are each independently selected from hydrogen, deuterium, optionally substituted C₁-C₆ alkyl, optionally substituted C₁-C₆ haloalkyl, or optionally substituted C₁-C₆ hydroxyalkyl.

In some embodiments, R⁸ and R⁹ are each independently selected from optionally substituted C₁-C₆ alkyl, C₁-C₆ haloalkyl, or C₁-C₆ hydroxyalkyl. In some embodiments, R⁸ and R⁹ are each independently selected from optionally substituted C₁-C₆ alkyl. In some embodiments, R⁸ and R⁹ are each independently methyl, ethyl, or propyl. In some embodiments, R⁸ and R⁹ are each independently methyl. In some embodiments, R⁸ and R⁹ are each independently ethyl. In some embodiments, R⁸ and R⁹ are each independently propyl.

In some embodiments, the second terminus comprising the structure of Formula (2-D):

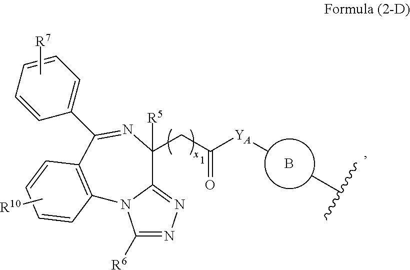

or a pharmaceutically acceptable salt thereof, wherein:
R¹⁰ is selected from hydrogen, deuterium, optionally substituted C₁-C₆ alkyl, optionally substituted C₁-C₆ haloalkyl, or optionally substituted C₁-C₆ hydroxyalkyl.

In some embodiments, R⁵ is C₁-C₆ alkyl. In some embodiments, R⁸ is methyl or ethyl. In some embodiments, R⁸ is methyl. In some embodiments, R⁵ is ethyl. In some embodiments, R⁵ is hydrogen.

In some embodiments, R⁷ is selected from hydrogen, halogen, optionally substituted C₁-C₆ alkyl, C₁-C₆ haloalkyl, or C₁-C₆ hydroxyalkyl. In some embodiments, R⁷ is halogen. In some embodiments, R⁷ is Br, Cl, or F. In some embodiments, R⁷ is Cl. In some embodiments, R⁷ is F. In some embodiments, R⁷ is Br.

In some embodiments, R⁷ is —NR^{7A}R^{7B}, wherein R^{7A} and R^{7B} are each independently hydrogen or optionally substituted C₁-C₆ alkyl.

In some embodiments, R¹⁰ is selected from optionally substituted C₁-C₆ alkyl, optionally substituted C₁-C₆ haloalkyl, or optionally substituted C₁-C₆ hydroxyalkyl. In some embodiments, R¹⁰ is selected from optionally substituted C₁-C₆ alkyl. In some embodiments, R¹⁰ is methyl, ethyl, or propyl. In some embodiments, R¹⁰ is methyl. In some embodiments, R¹⁰ is optionally substituted C₁_₆ hydroxyalkyl. In some embodiments, R¹⁰ is —OMe.

In some embodiments, R⁶ is selected from optionally substituted C₁-C₆ alkyl, optionally substituted C₁-C₆ haloalkyl, or optionally substituted C₁-C₆ hydroxyalkyl. In some embodiments, R⁶ is an optionally substituted C₁-C₆ alkyl. In some embodiments, R⁶ is methyl, ethyl, or propyl. In some embodiments, R⁶ is methyl. In some embodiments, R⁶ is ethyl. In some embodiments, R⁶ is propyl. In some embodiments, R⁶ is hydrogen.

In some embodiments, Y_{A} is —NH—. In some embodiments, Y_{A} is —O—.

In some embodiments, Y_{A} is NH and x₁ is 1.

In some embodiments, x₁ is an integer from 1-5, 1-4, 1-3, or 1-2. In some embodiments, x₁ is 1. In some embodiments, x₁ is 2.

In some embodiments, Ring B is an optionally substituted 6-membered monocyclic aryl or heteroaryl, each of which is optionally substituted with alkyl, amino, halogen, hydroxy, hydroxyalkyl, or PEG. In some embodiments Ring B is phenyl. In some embodiments, Ring B is 6-membered monocyclic heteroaryl. In some embodiments, Ring B is pyridine or pyrimidine. In some embodiments, ring B is absent.

In some embodiments, the second terminus comprises the structure of Formula (2-E), or a pharmaceutically acceptable salt thereof:

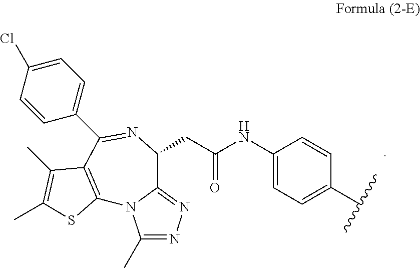 [SMILES: *c1ccc(NC(=O)C[C@H]2N=C(c3ccc(Cl)cc3)c3c(sc(C)c3C)-n3c(C)nnc32)cc1]

In some embodiments, the second terminus comprises the structure of Formula (2-F), or a pharmaceutically acceptable salt thereof:

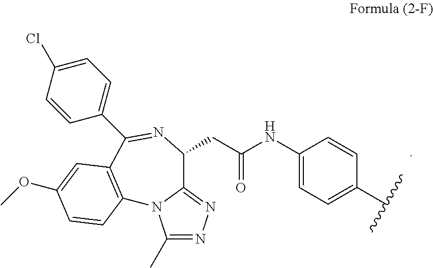 [SMILES: *c1ccc(NC(=O)C[C@H]2N=C(c3ccc(Cl)cc3)c3cc(OC)ccc3-n3c(C)nnc32)cc1]

In some embodiments, the second terminus comprises the structure of Formula (2-G), or a pharmaceutically acceptable salt thereof:

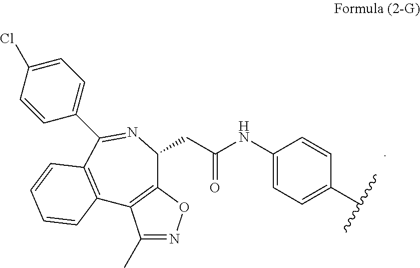 [SMILES: *c1ccc(NC(=O)C[C@H]2N=C(c3ccc(Cl)cc3)c3ccccc3-c3c(C)noc32)cc1]

In some embodiments, the second terminus comprises the structure of Formula (3-A):

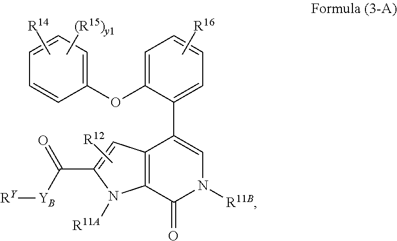

or a pharmaceutically acceptable salt thereof, wherein,
Y_{B} is —CH₂NH—, —CH₂O—, —NH—, or —O—;
R^{11A} and R^{11B} are each independently hydrogen, deuterium, or optionally substituted C₁-C₆ alkyl;
R¹² is hydrogen, halogen, —OH, optionally substituted C₁-C₆ alkyl, optionally substituted C₁-C₆ haloalkyl, or optionally substituted C₁-C₆ hydroxyalkyl;
R¹⁴ and R¹⁵ are each independently hydrogen, deuterium, halogen, —CN, —NO₂, optionally substituted C₁-C₆ alkyl, optionally substituted C₁-C₆ haloalkyl, or optionally substituted C₁-C₆ hydroxyalkyl; or R¹⁴ is —NR^{A}R^{B};
R¹⁶ is optionally substituted C₁-C₆ alkyl, optionally substituted C₁-C₆ heteroalkyl, optionally substituted C₂-C₆ alkenyl, optionally substituted C₂-C₆ alkynyl, C₁-C₆ hydroxyalkyl, —SO₂R^{A}, or —NHSO₂R^{A};
R^{Y} is hydrogen, optionally substituted C₁-C₆ alkyl, optionally substituted C₁-C₆ haloalkyl, or optionally substituted 5-6-membered monocyclic aryl or heteroaryl;
each R^{A} and R^{B} is independently hydrogen, deuterium, optionally substituted C₁-C₆ alkyl, or optionally substituted C₁-C₆ heteroalkyl; and
y₁ is 1-3; and wherein the attachment to the linker is at either R¹⁴ or at R^{Y}.

In some embodiments, the second terminus comprises the structure of Formula (3-B):

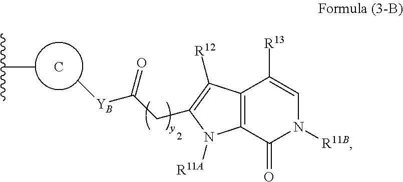

or a pharmaceutically acceptable salt thereof, wherein,
Ring C is absent, optionally substituted 5 to 6-membered monocyclic aryl or heteroaryl or 4 to 8-membered heterocycle;
Y_{B} is —NH—, —CH₂NH—, —CH₂O—, or —O—;
R^{11A} and R^{11B} are each independently hydrogen, deuterium, or optionally substituted C₁-C₆ alkyl;
R¹² is hydrogen, deuterium, optionally substituted C₁-C₆ alkyl, C(O)R^{A}, or C(O)NR^{A}R^{B}; wherein
   each R^{A} and R^{B} is independently hydrogen, deuterium, optionally substituted C₁-C₆ alkyl, or optionally substituted C₁-C₆ heteroalkyl;
R¹³ is hydrogen, substituted aryl, substituted heteroaryl, or substituted oxydibenzene; and
y₂ is an integer from 0-2.

In some embodiments, y₂ is 0. In some embodiments, y₂ is 1. In some embodiments, y₂ is 2.

In some embodiments, R¹³ is substituted aryl or substituted heteroaryl. In some embodiments, R¹³ is hydrogen.

In some embodiments, R¹³ is substituted oxydibenzene.

In some embodiments, R¹³ is

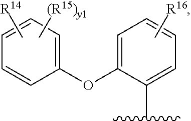

wherein
R¹⁴ and R¹⁵ are each independently hydrogen, deuterium, halogen, —CN, —NO₂, optionally substituted C₁-C₆ alkyl, optionally substituted C₁-C₆ haloalkyl, or optionally substituted C₁-C₆ hydroxyalkyl;
or R¹⁴ is —NR^{A}R^{B};
R¹⁶ is optionally substituted C₁-C₆ alkyl, optionally substituted C₁-C₆ heteroalkyl, optionally substituted C₂-C₆ alkenyl, optionally substituted C₂-C₆ alkynyl, C₁-C₆ hydroxyalkyl, —SO₂R^{A}, or —NHSO₂R^{A};
each R^{A} and R^{B} is independently hydrogen, deuterium, optionally substituted C₁-C₆ alkyl, or optionally substituted C₁-C₆ heteroalkyl; and
y₁ is 1-3.

In some embodiments, the second terminus comprises the structure of Formula (3-C):

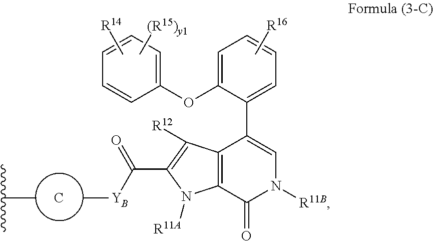

or a pharmaceutically acceptable salt thereof, wherein:
Ring C is absent, optionally substituted 5 to 6-membered monocyclic aryl or heteroaryl or 4 to 8-membered heterocycle;
Y_{B} is —NH—, —CH₂NH—, —CH₂O—, or —O—;
R¹⁴ and R¹⁵ are each independently hydrogen, deuterium, halogen, —CN, —NO₂, optionally substituted C₁-C₆ alkyl, optionally substituted C₁-C₆ haloalkyl, or optionally substituted C₁-C₆ hydroxyalkyl;
or R¹⁴ is —NR^{A}R^{B}, wherein
   each R^{A} and R^{B} is independently hydrogen, deuterium, optionally substituted C₁-C₆ alkyl, or optionally substituted C₁-C₆ heteroalkyl;
R¹⁶ is optionally substituted C₁-C₆ alkyl, optionally substituted C₁-C₆ heteroalkyl, optionally substituted C₂-C₆ alkenyl, optionally substituted C₂-C₆ alkynyl, C₁-C₆ hydroxyalkyl, —SO₂R^{A}, or —NHSO₂R^{A}; and
y₁ is an integer from 1-3.

In some embodiments, Y_{B} is —NH—. In some embodiments, Y_{B} is —CH₂NH—. In some embodiments, Y_{B} is —CH₂O—. In some embodiments, Y_{B} is —O—.

In some embodiments, Ring C is an optionally substituted 5 or 6-membered monocyclic aryl or heteroaryl, each of which is optionally substituted with alkyl, amino, halogen, hydroxy, hydroxyalkyl, or PEG.

In some embodiments, Ring C is phenyl. In some embodiments, Ring C is a 6-membered heteroaryl. In some embodiments, Ring C is pyridine, pyrazine, or triazine. In some embodiments, Ring C is pyridine. In some embodiments, Ring C is pyrazine. In some embodiments, Ring C is triazine. In some embodiments, Ring C is a 5-membered heteroaryl. In some embodiments, Ring C is a pyrazole. In some embodiments, Ring C is a triazole, pyrrole, imidazole, oxazole, oxadiazole, thiazole, or thiadiazole. In some embodiments, Ring C is a triazole. In some embodiments, Ring C is an imidazole or pyrrole. In some embodiments, an oxazole or oxadiazole. In some embodiments, Ring C is a thiazole or thiadiazole.

In some embodiments, Ring C is absent.

In some embodiments, the second terminus comprises the structure of Formula (3-D), or a pharmaceutically acceptable salt thereof:

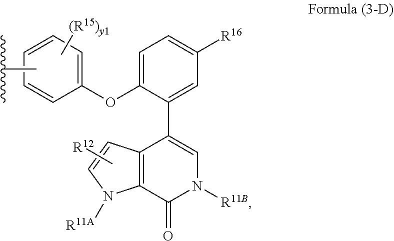

or a pharmaceutically acceptable salt or solvate thereof, wherein.
R^{11A} and R^{11B} are each independently hydrogen, deuterium, or optionally substituted C₁-C₆ alkyl;
R¹² is hydrogen, deuterium, optionally substituted C₁-C₆ alkyl, C(O)R^{A}, or C(O)NR^{A}R^{B};
each R¹⁵ is independently hydrogen, deuterium, halogen, —CN, —NO₂, optionally substituted C₁-C₆ alkyl, optionally substituted C₁-C₆ haloalkyl, or optionally substituted C₁-C₆ hydroxyalkyl;
R¹⁶ is optionally substituted C₁-C₆ alkyl, optionally substituted C₁-C₆ heteroalkyl, optionally substituted C₂-C₆ alkenyl, optionally substituted C₂-C₆ alkynyl, optionally substituted C₁-C₆ hydroxyalkyl, —SO₂R^{A}—, or —NHSO₂R^{A}; wherein
   each R^{A} and R^{B} is independently hydrogen, deuterium, optionally substituted C₁-C₆ alkyl, or optionally substituted C₁-C₆ heteroalkyl; and
y₁ is an integer from 1-3.

In some embodiments, R^{11A} and R^{11B} are each independently optionally substituted C₁-C₆ alkyl. In some embodiments, R^{11A} and R^{11B} are each independently methyl, ethyl, propyl, or tert-butyl. In some embodiments, R^{11A} and R^{11B} are each independently methyl. In some embodiments, R^{11A} and R^{11B} are each independently hydrogen.

In some embodiments, R^{11A} is C₁-C₆ alkyl, optionally substituted with haloalkyl or phosphorous hydroxide. In some embodiments, R^{11A is C}₁-C₆ alkyl substituted with —OP(O)(OH)₂. In some embodiments, R^{11A} is unsubstituted C₁-C₆ alkyl. In some embodiments, R^{11A} is methyl, ethyl, or tert-butyl. In some embodiments, R^{11A} is methyl. In some embodiments, R^{11A} is hydrogen.

In some embodiments, R¹² is optionally substituted C₁-C₆ alkyl. In some embodiments, R¹² is hydrogen.

In some embodiments, R¹² is C(O)R^{A} or C(O)NR^{A}R^{B}. In some embodiments, R¹² is C(O)NR^{A}R^{B}, wherein R^{A} and R^{B} are each independently hydrogen or optionally substituted C₁-C₆ alkyl.

In some embodiments, R¹⁴ and R¹⁵ are each independently hydrogen, —CN, or —NO₂. In some embodiments, R¹⁴ and R¹⁵ are each independently halogen or optionally substituted C₁-C₆ alkyl. In some embodiments, R¹⁴ and R¹⁵ are each independently Br, Cl, F, methyl, or ethyl. In some embodiments, R¹⁴ and R¹⁵ are each independently F or methyl.

In some embodiments, R¹⁶ is optionally substituted optionally substituted C₁-C₆ alkyl, optionally substituted C₁-C₆ heteroalkyl, optionally substituted C₂-C₆ alkenyl, optionally substituted C₂-C₆ alkynyl, or optionally substituted C₁-C₆ hydroxyalkyl, each of which is optionally substituted with amido, alkyl, alkynyl, azido, amino, halogen, haloalkyl, hydroxy, nitro, oxo (═O), phosphorous hydroxide, or PEG.

In some embodiments, R¹⁶ is optionally substituted optionally substituted C₁-C₆ alkyl, optionally substituted C₁-C₆ heteroalkyl, or optionally substituted C₁-C₆ hydroxyalkyl. In some embodiments, R¹⁶ is C₁-C₆ alkyl or C₁-C₆ heteroalkyl, each or which optionally substituted with —CN, —NH₂, —N₃, —OH, CF₃, or —OP(O)(OH)₂.

In some embodiments, R¹⁶ is —SO₂R^{A}, wherein R^{A} is C₁-C₆ alkyl. In some embodiments, R¹⁶ is —SO₂Et. In some embodiments, R¹⁶ is —SO₂Me.

In some embodiments, R¹⁶ is —NHSO₂R^{A}, wherein R^{A} is C₁-C₆ alkyl. In some embodiments, R¹⁶ is —NHSO₂Et. In some embodiments, R¹⁶ is —NHSO₂Me.

In some embodiments, y₁ is 1. In some embodiments, y₁ is 2. In some embodiments, y₁ is 3.

In some embodiments, the second terminus comprises the structure of Formula (3-E), or a pharmaceutically acceptable salt thereof:

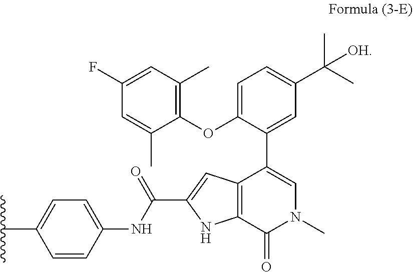 [SMILES: *c1ccc(NC(=O)c2cc3c(-c4cc(C(C)(C)O)ccc4Oc4c(C)cc(F)cc4C)cn(C)c(=O)c3[nH]2)cc1]

In some embodiments, the second terminus comprising the structure of Formula (3-F), or a pharmaceutically acceptable salt thereof:

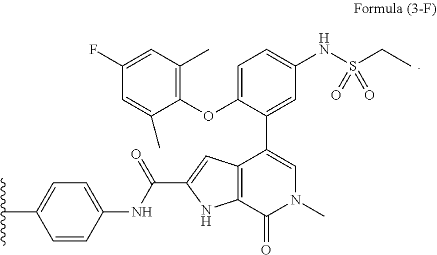 [SMILES: *c1ccc(NC(=O)c2cc3c(-c4cc(NS(=O)(=O)CC)ccc4Oc4c(C)cc(F)cc4C)cn(C)c(=O)c3[nH]2)cc1]

In some embodiments, the second terminus comprising the structure of Formula (3-G) or Formula (3-H), or a pharmaceutically acceptable salt thereof:

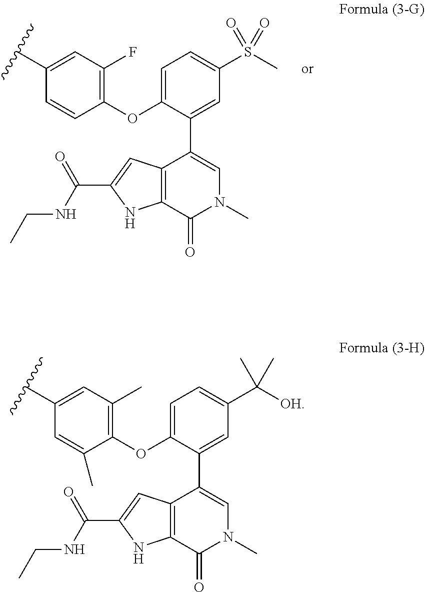

In some embodiments, the second terminus comprises the structure of Formula (4-A):

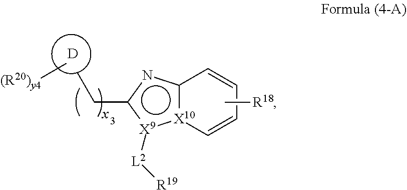

or a pharmaceutically acceptable salt thereof, wherein;
Ring D is absent, phenyl, or 5 to 6-membered heteroaryl;
X⁹ and X¹⁰ are each independently C or N, wherein one of X⁹ or X¹⁰ is N;
L² is absent, optionally substituted alkylene, —O—, or —NR^{D}—, wherein
   R^{D} is hydrogen, deuterium, or optionally substituted C₁-C₃ alkyl;
R¹⁸ is an optionally substituted 5 to 6-membered heteroaryl;
R¹⁹ is an optionally substituted C₃-C₅ cycloalkyl or optionally substituted 4 to 7-membered heteroaryl;
each R²⁰ is independently hydrogen, deuterium, halogen, —CN, —NO₂, optionally substituted C₁-C₆ alkyl, optionally substituted C₁-C₆ haloalkyl, or optionally substituted C₁-C₆ hydroxyalkyl;
x₃ is an integer from 1-3; and
y₃ is an integer from 1-4; and
   wherein the attachment to the linker is at either R¹⁹ or at one of R²⁰.

In some embodiments, attachment to the linker is at R¹⁹.

In some embodiments, attachment to the linker is at one of R²⁰.

In some embodiments, the second terminus comprises the structure of Formula (4-B):

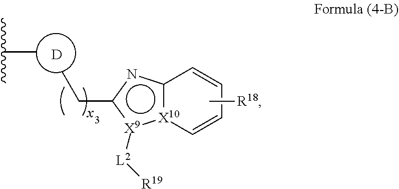

or a pharmaceutically acceptable salt thereof, wherein;
Ring D is absent, optionally substituted phenyl, or optionally substituted 5 to 6-membered heteroaryl;
X⁹ and X¹⁰ are each independently C or N, wherein one of X⁹ or X¹⁰ is N;
L² is absent, optionally substituted alkylene, —O—, or —NR^{D}—, wherein
   R^{D} is hydrogen, deuterium, or optionally substituted C₁-C₃ alkyl;
R¹⁸ is an optionally substituted 5 to 6-membered heteroaryl;
R¹⁹ is an optionally substituted C₃-C₈ cycloalkyl or optionally substituted 4 to 7-membered heteroaryl; and
x₃ is an integer from 1-3.

In some embodiments, X⁹ is N; and X¹⁰ is C. In some embodiments, X⁹ is C; and X¹⁰ is N.

In some embodiments, the second terminus comprises the structure of Formula (4-C), or a pharmaceutically acceptable salt thereof:

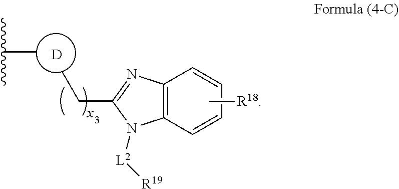

In some embodiments, Ring D is an optionally substituted monocyclic 6-membered aryl or 5 to 6-membered heteroaryl. In some embodiments, Ring D is an optionally substituted monocyclic 6-membered aryl. In some embodiments, Ring D is an optionally substituted phenyl.

In some embodiments, R¹⁹ is an optionally substituted C₃-C₈ cycloalkyl. In some embodiments, R¹⁹ is optionally substituted 4 to 7-membered heteroaryl.

In some embodiments, the second terminus comprises the structure of Formula (4-D):

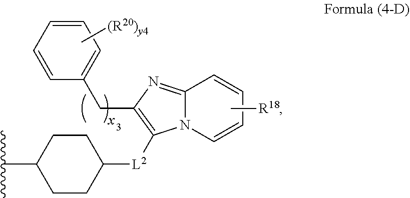

or a pharmaceutically acceptable salt thereof, wherein;
L² is an optionally substituted alkylene, —O—, or —NR^{D}—, wherein
   R^{D} is hydrogen, deuterium, or optionally substituted C₁-C₃ alkyl;
R¹⁸ is an optionally substituted 5 to 6-membered heteroaryl;
R²⁰ is hydrogen, deuterium, halogen, —CN, —NO₂, optionally substituted C₁-C₆ alkyl, optionally substituted C₁-C₆ haloalkyl, or optionally substituted C₁-C₆ hydroxyalkyl;
x₃ is an integer from 1-3; and
y₃ is an integer from 1-4.

In some embodiments, L² is an optionally substituted alkylene. In some embodiments, L² is C₂-C₄ alkylene, optionally substituted with one or more C₁-C₃ alkyl. In some embodiments, L² is absent.

In some embodiments, L² is —NR^{D}—. In some embodiments, L² is —NH—.

In some embodiments, R¹⁸ is an optionally substituted 5-membered heteroaryl. In some embodiments, R¹⁸ is optionally substituted oxazole, oxadiazole, thiazole, thiadiazole, pyrrole, or pyrazole. In some embodiments, R¹⁸ is optionally substituted oxazole.

In some embodiments, R²⁰ is halogen, —CN, —NO₂, optionally substituted C₁-C₆ alkyl, optionally substituted C₁-C₆ haloalkyl, or optionally substituted C₁-C₆ hydroxyalkyl.

In some embodiments, x₃ is 1. In some embodiments, x₃ is 2. In some embodiments, x₃ is 3.

In some embodiments, y₄ is 1 or 2. In some embodiments, y₄ is 1. In some embodiments, y₄ is 2. In some embodiments, y₄ is 3. In some embodiments, y₄ is 4.

In some embodiments, the second terminus comprises the structure of Formula (4-E) or Formula (4-F), or a pharmaceutically acceptable salt thereof:

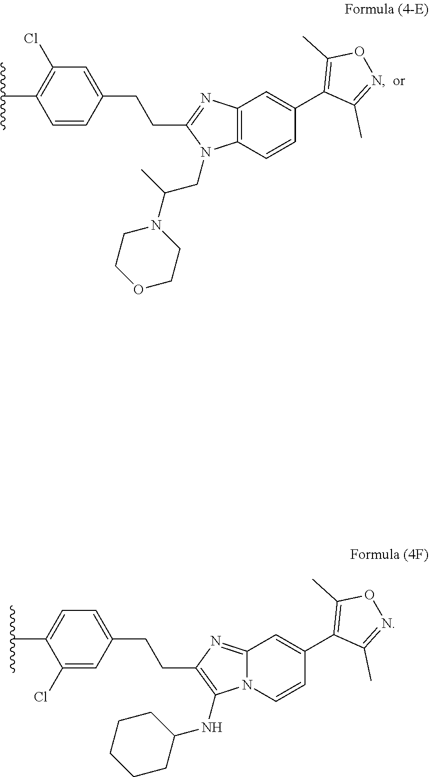 [SMILES: *c1ccc(CCc2nc3cc(-c4c(C)noc4C)ccc3n2CC(C)N2CCOCC2)cc1Cl ; *c1ccc(CCc2nc3cc(-c4c(C)noc4C)ccn3c2NC2CCCCC2)cc1Cl]

In some embodiments, the second terminus comprises the structure of Formula (4-G), or a pharmaceutically acceptable salt thereof:

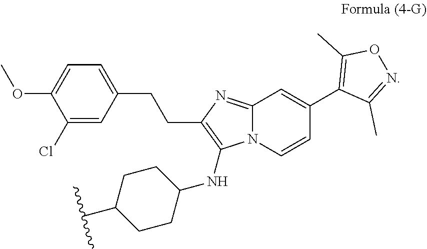 [SMILES: *C1CCC(Nc2c(CCc3ccc(OC)c(Cl)c3)nc3cc(-c4c(C)noc4C)ccn23)CC1]

In some embodiments, the second terminus comprises the structure of Formula (5-A):

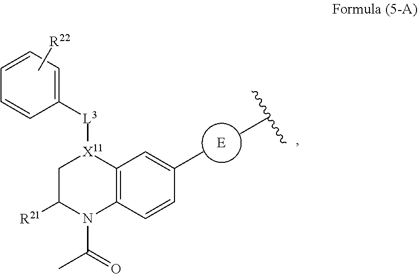

or a pharmaceutically acceptable salt thereof, wherein;
Ring E is absent or an optionally substituted phenyl or optionally substituted 5 to 6-membered heteroaryl;
X¹¹ is CH or N;
L³ is —NR^{E}— or —CR^{E}R^{E}—, wherein
   each R^{E} is independently hydrogen, deuterium, or optionally substituted C₁-C₃ alkyl;
R²¹ is C₁-C₆ alkyl or C₃-C₆ cycloalkyl; and
R²² is halogen, CN, NO₂, optionally substituted C₁-C₆ alkyl, optionally substituted C₁-C₆ haloalkyl, or optionally substituted C₁-C₆ hydroxyalkyl.

In some embodiments, Ring E is absent. In some embodiments, Ring E is an optionally substituted phenyl. In some embodiments, Ring E is an optionally substituted 5 to 6-membered heteroaryl. In some embodiments, Ring E is a 5-membered heteroaryl. In some embodiments, Ring E is a 6-membered heteroaryl.

In some embodiments, X¹¹ is CH and L³ is —NR^{E}—. In some embodiments, X¹¹ is N and L³ is —CR^{E}R^{E}—.

In some embodiments, R²¹ is C₁-C₆ alkyl. In some embodiments, R²¹ is methyl.

In some embodiments, R²² is halogen, optionally substituted C₁-C₆ alkyl, optionally substituted C₁-C₆ haloalkyl, or optionally substituted C₁-C₆ hydroxyalkyl. In some embodiments, R²² is CN, F, Cl, Br, or methyl.

In some embodiments, the second terminus comprises the structure of Formula (5-B), or a pharmaceutically acceptable salt thereof:

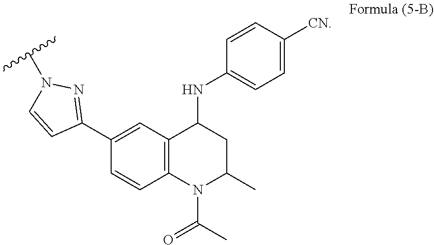 [SMILES: *n1ccc(-c2ccc3c(c2)C(Nc2ccc(C#N)cc2)CC(C)N3C(C)=O)n1]

In some embodiments, the second terminus comprises the structure of Formula (6-A):

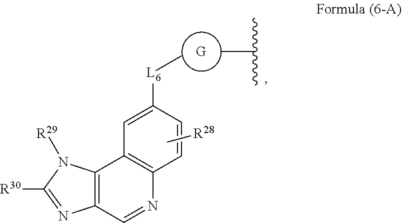

or a pharmaceutically acceptable salt thereof, wherein;
Ring G is an optionally substituted C₃-C₆ cycloalkyl or optionally substituted 4 to 6-membered heterocycloalkyl;
L⁶ is —O-(optionally substituted alkylene);
R²⁸ is an optionally substituted 5 to 6-membered heteroaryl;
R²⁹ is optionally substituted C₁-C₆ alkyl(C₆-C₁₀ aryl) or optionally substituted C₁-C₆ alkyl(6 to 10-membered heteroaryl); and
R³⁰ is optionally substituted C₁-C₆ alkyl, optionally substituted C₁-C₆ haloalkyl, or optionally substituted C₁-C₆ hydroxyalkyl.

In some embodiments, the second terminus comprises the structure of Formula (7-A):

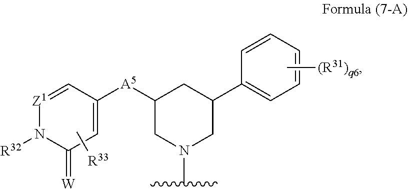

or a pharmaceutically acceptable salt thereof, wherein,
A⁵ is —O—, —NH—, or —CH₂—;
Z¹ is CH or N;
W is O or S;
each R³¹ is independently hydrogen, halogen, —OH, —CN, —NO₂, —NH₂, optionally substituted C₁-C₁₀ alkyl, optionally substituted C₁-C₁₀ haloalkyl, optionally substituted C₁-C₁₀ hydroxyalkyl, optionally substituted C₂-C₁₀ alkenyl, optionally substituted C₂-C₁₀ alkynyl, optionally substituted C₃-C₈ cycloalkyl, or optionally substituted 3 to 8-membered heterocycloalkyl;
or two R³¹ together with the atoms to which they are attached join together to form an optionally substituted C₅-C₈ cycloalkyl or optionally substituted 5 to 8-membered heterocycloalkyl;
R³² is hydrogen or optionally substituted C₁-C₁₀ alkyl;
R³³ is hydrogen, halogen, —OH, —CN, —NO₂, —NH₂, optionally substituted C₁-C₁₀ alkyl, optionally substituted C₁-C₁₀ haloalkyl, or optionally substituted C₁-C₁₀ hydroxyalkyl; and
q₆ is 0-4.

In some embodiments, Z¹ is CH. In some embodiments, Z¹ is N.

In some embodiments, W is O. In some embodiments, W is S.

In some embodiments, each R³¹ is independently an optionally substituted C₁-C₁₀ alkyl, optionally substituted C₁-C₁₀ haloalkyl, or optionally substituted C₁-C₁₀ hydroxyalkyl. In some embodiments, each R³¹ is independently an optionally substituted C₃-C₈-cycloalkyl or optionally substituted 3 to 8-membered heterocycloalkyl. In some embodiments, each R³¹ is independently hydrogen, halogen, —OH, —CN, —NO₂, or —NH₂. In some embodiments, each R³¹ is hydrogen.

In some embodiments, R³² is an optionally substituted C₁-C₁₀ alkyl. In some embodiments, R³² is methyl. In some embodiments, R³² is hydrogen.

In some embodiments, R³³ is hydrogen, halogen, —OH, —CN, —NO₂, or —NH₂. In some embodiments, R³³ is an optionally substituted C₁-C₁₀ alkyl, optionally substituted C₁-C₁₀ haloalkyl, or optionally substituted C₁-C₁₀ hydroxyalkyl.

In some embodiments, the second terminus comprises the structure of Formula (7-B), or a pharmaceutically acceptable salt thereof:

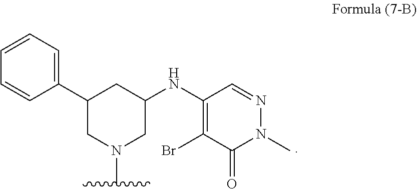 [SMILES: *N1CC(Nc2cnn(C)c(=O)c2Br)CC(c2ccccc2)C1]

In some embodiments, the second terminus comprises the structure of Formula (8-A):

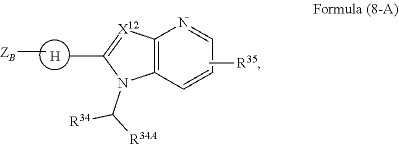

or a pharmaceutically acceptable salt thereof, wherein,
Ring H is an optionally substituted phenyl or optionally substituted 6-membered heteroaryl;

### or Ring H is

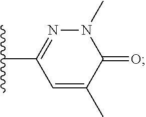 [SMILES: *c1cc(C)c(=O)n(C)n1] [0346] Z_{B} is absent or an optionally substituted phenyl formamide;
X¹² is CH or N;
R³⁴ is an optionally substituted phenyl or optionally substituted 6-membered heteroaryl;
R^{34A} is hydrogen, halogen, or optionally substituted C₁-C₃ alkyl;
R³³ is independently halogen, optionally substituted C₁-C₁₀ alkyl, or optionally substituted 5-membered heteroaryl; and
   wherein the attachment to the linker is at R³⁵, Z_{B}, or Ring H.

In some embodiments, Ring H is an optionally substituted phenyl. In some embodiments, Ring H is an optionally substituted 6-membered heteroaryl.

In some embodiments, Ring H is

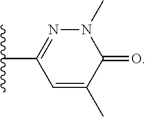 [SMILES: *c1cc(C)c(=O)n(C)n1]

In some embodiments, Z_{B} is absent. In some embodiments, Z_{B} is an optionally substituted phenyl formamide. In some embodiments, Z_{B} is —C(O)NH-phenyl.

In some embodiments, X¹² is CH. In some embodiments, X¹² is N.

In some embodiments, R³⁴ is an optionally substituted phenyl. In some embodiments, R³⁴ is an optionally substituted 6-membered heteroaryl.

In some embodiments, R^{34A} is hydrogen or halogen. In some embodiments, R^{34A} is an optionally substituted C₁-C₃ alkyl. In some embodiments, R^{34A} is methyl.

In some embodiments, Formula (8-A) is attachment to the linker is at R³⁵. In some embodiments, Formula (8-A) is attached to the linker at Z_{B}. In some embodiments, Formula (8-A) is attached to the linker at Ring H.

In some embodiments, the second terminus comprises the structure of Formula (8-B) or Formula (8-C), or a pharmaceutically acceptable salt thereof:

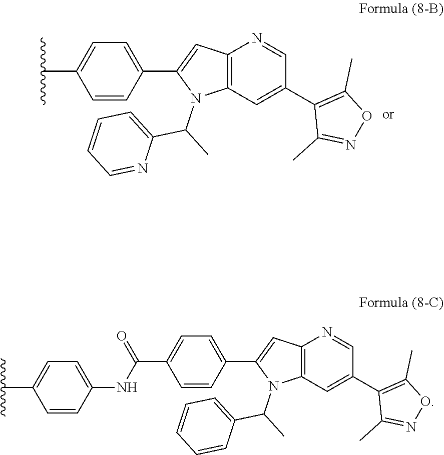 [SMILES: *c1ccc(-c2cc3ncc(-c4c(C)noc4C)cc3n2C(C)c2ccccn2)cc1 ; *c1ccc(NC(=O)c2ccc(-c3cc4ncc(-c5c(C)noc5C)cc4n3C(C)c3ccccc3)cc2)cc1]

In some embodiments, the second terminus comprises the structure of Formula (8-D), or a pharmaceutically acceptable salt thereof:

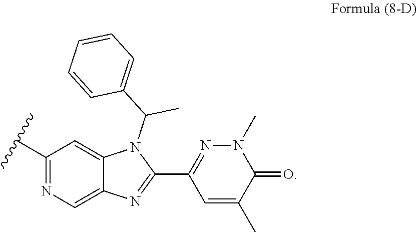 [SMILES: *c1cc2c(cn1)nc(-c1cc(C)c(=O)n(C)n1)n2C(C)c1ccccc1]

In some embodiments, the second terminus comprises the structure of Formula (9-A), or a pharmaceutically acceptable salt thereof:

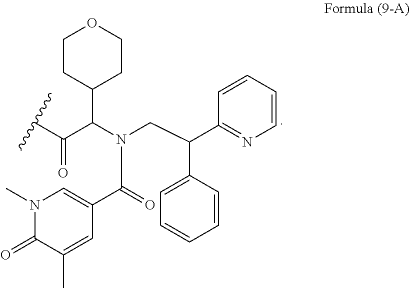 [SMILES: *C(=O)C(C1CCOCC1)N(CC(c1ccccc1)c1ccccn1)C(=O)c1cc(C)c(=O)n(C)c1]

In some embodiments, the second terminus comprises the structure of Formula (10-A) or Formula (10-B), or a pharmaceutically acceptable salt thereof:

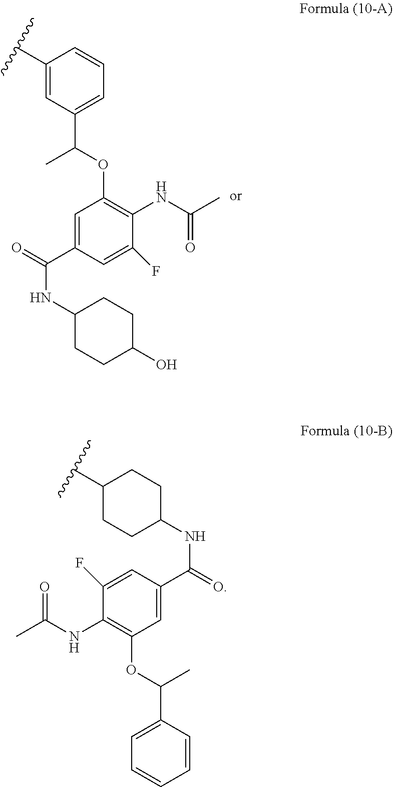

In some embodiments, the second terminus comprises the structure of Formula (11-A), or a pharmaceutically acceptable salt thereof:

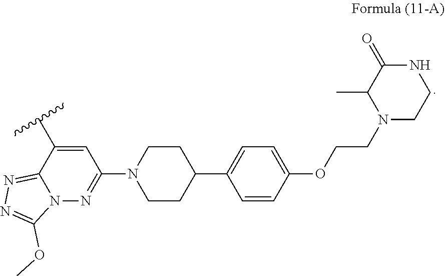 [SMILES: *c1cc(N2CCC(c3ccc(OCCN4CCNC(=O)C4C)cc3)CC2)nn2c(OC)nnc12]

In some embodiments, the second terminus is selected from the group consisting of:

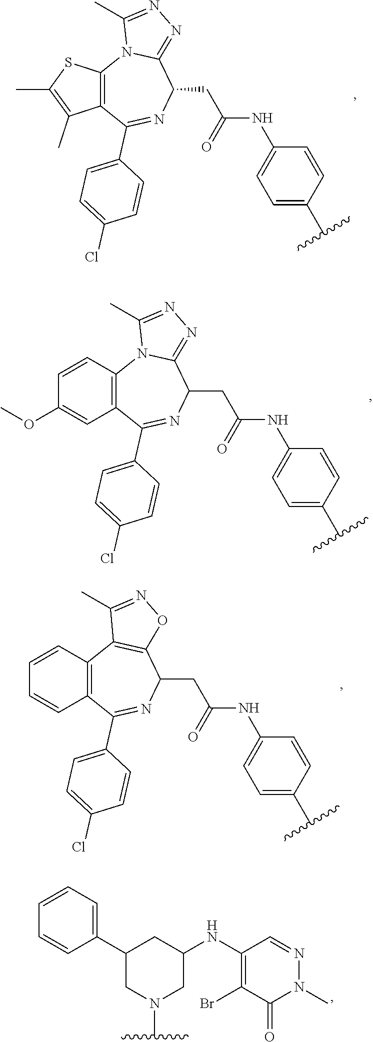

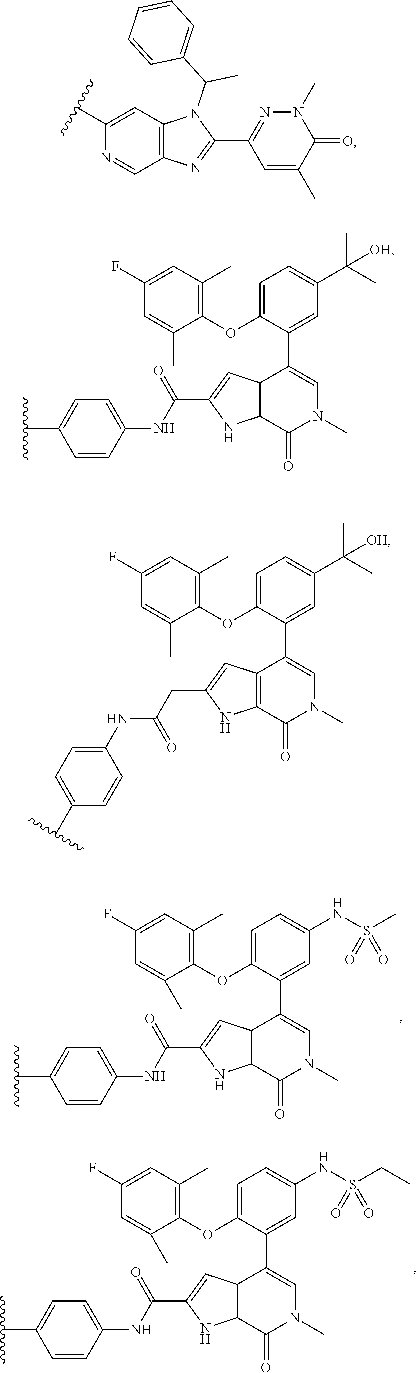

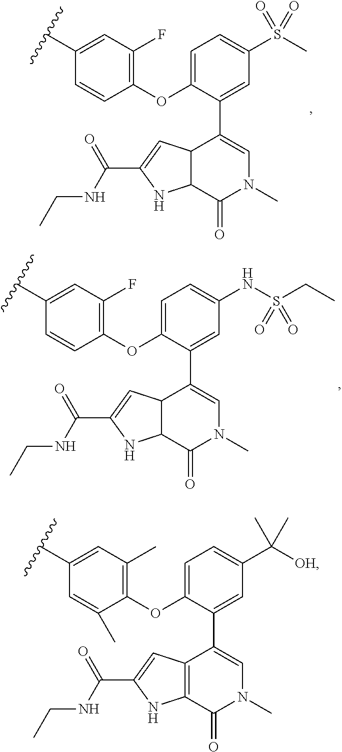

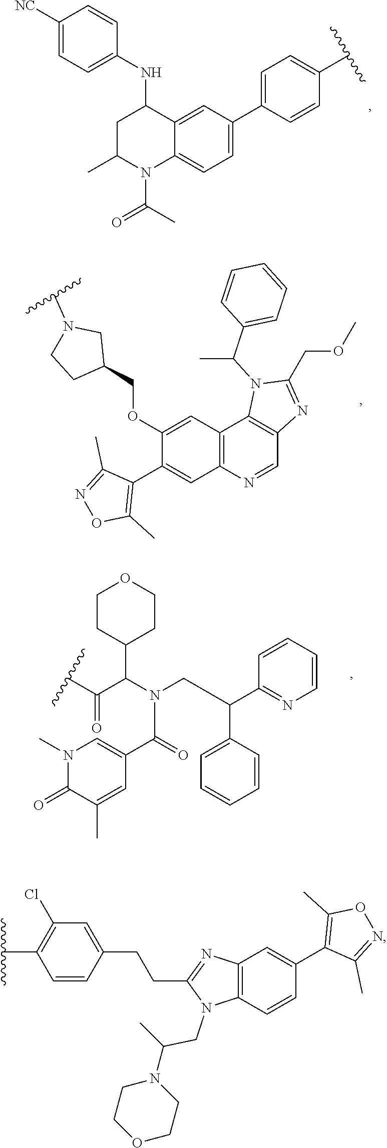 [SMILES: *c1ccc(-c2ccc3c(c2)C(Nc2ccc(C#N)cc2)CC(C)N3C(C)=O)cc1 ; *N1CC[C@H](COc2cc3c(cc2-c2c(C)noc2C)ncc2nc(COC)n(C(C)c4ccccc4)c23)C1 ; *C(=O)C(C1CCOCC1)N(CC(c1ccccc1)c1ccccn1)C(=O)c1cc(C)c(=O)n(C)c1 ; *c1ccc(CCc2nc3cc(-c4c(C)noc4C)ccc3n2CC(C)N2CCOCC2)cc1Cl]

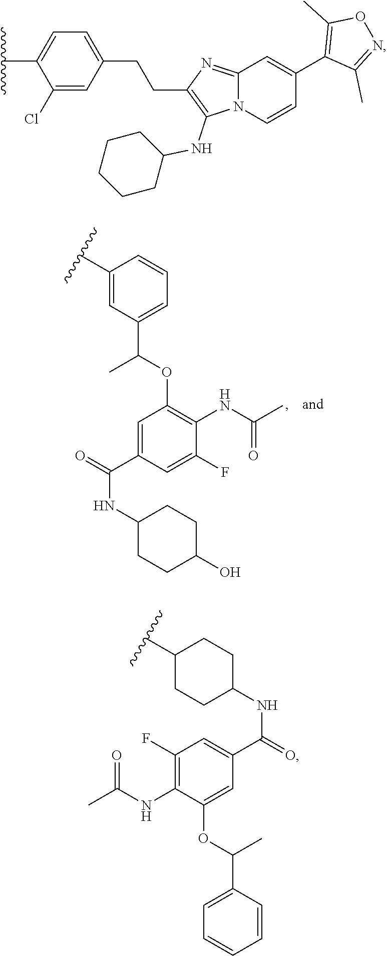 [SMILES: *c1ccc(CCc2nc3cc(-c4c(C)noc4C)ccn3c2NC2CCCCC2)cc1Cl ; *c1cccc(C(C)Oc2cc(C(=O)NC3CCC(O)CC3)cc(F)c2NC(C)=O)c1 ; *C1CCC(NC(=O)c2cc(F)c(NC(C)=O)c(OC(C)c3ccccc3)c2)CC1]

or a pharmaceutically acceptable salt thereof.

In some embodiments, the second terminus is selected from a moiety described in Table 2, or a pharmaceutically acceptable salt thereof.

TABLE 2
Exemplary bromodomain binding moieties.

| Structure | Binder |
|---|---|
| 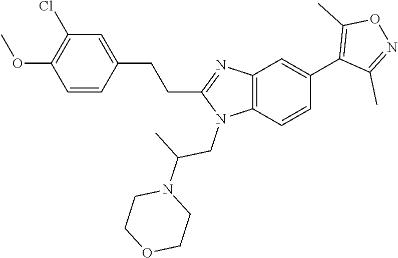 [SMILES: COc1ccc(CCc2nc3cc(-c4c(C)noc4C)ccc3n2CC(C)N2CCOCC2)cc1Cl] | CBP/P300 |
| 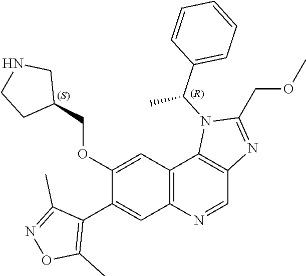 | BET(BD1) |
| 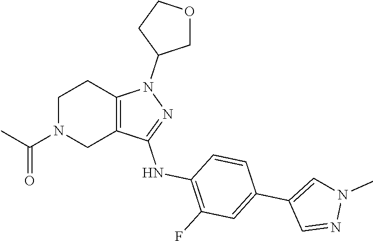 [SMILES: CC(=O)N1CCc2c(c(Nc3ccc(-c4cnn(C)c4)cc3F)nn2C2CCOC2)C1] | PCAF |
| 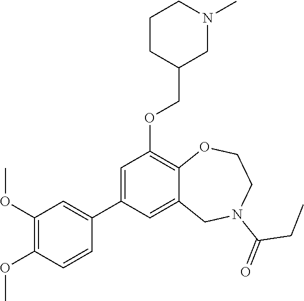 [SMILES: CCC(=O)N1CCOc2c(cc(-c3ccc(OC)c(OC)c3)cc2OCC2CCCN(C)C2)C1] | CBP/P300 |
| 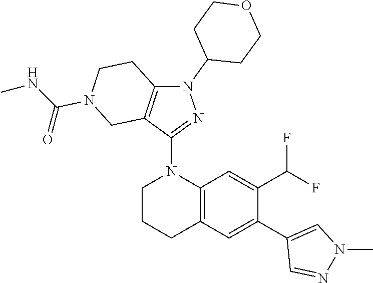 | CBP/P300 |
| 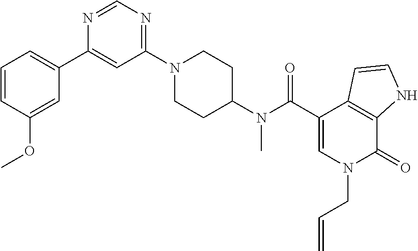 [SMILES: C=CCn1cc(C(=O)N(C)C2CCN(c3cc(-c4cccc(OC)c4)ncn3)CC2)c2cc[nH]c2c1=O] | CECR2 |
| 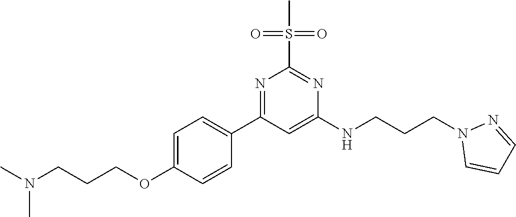 [SMILES: CN(C)CCCOc1ccc(-c2cc(NCCCn3cccn3)nc(S(C)(=O)=O)n2)cc1] | BPTF |
| 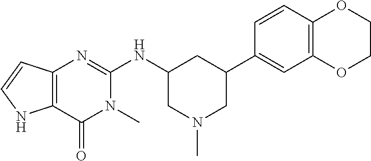 [SMILES: CN1CC(Nc2nc3cc[nH]c3c(=O)n2C)CC(c2ccc3c(c2)OCCO3)C1] | PCAF |
| 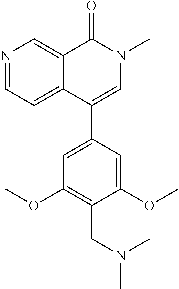 [SMILES: COc1cc(-c2cn(C)c(=O)c3cnccc23)cc(OC)c1CN(C)C] | BRD7/9 |
| 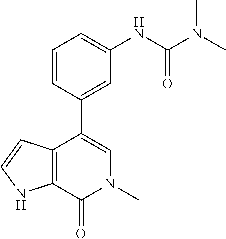 [SMILES: CN(C)C(=O)Nc1cccc(-c2cn(C)c(=O)c3[nH]ccc23)c1] | TAF1 |
| 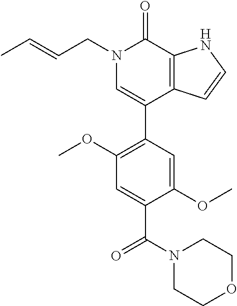 [SMILES: C/C=C/Cn1cc(-c2cc(OC)c(C(=O)N3CCOCC3)cc2OC)c2cc[nH]c2c1=O] | BRD7/9 |
| 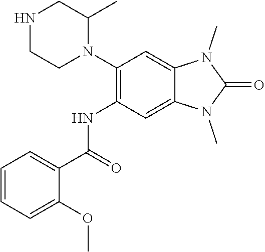 [SMILES: COc1ccccc1C(=O)Nc1cc2c(cc1N1CCNCC1C)n(C)c(=O)n2C] | BRPF |
| 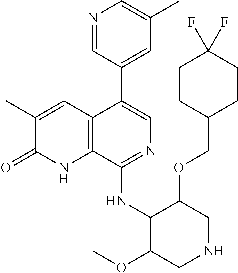 [SMILES: COC1CNCC(OCC2CCC(F)(F)CC2)C1Nc1ncc(-c2cncc(C)c2)c2cc(C)c(=O)[nH]c12] | ATAD2/ATAD2B |
| 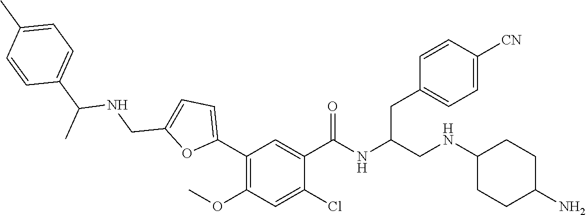 [SMILES: COc1cc(Cl)c(C(=O)NC(CNC2CCC(N)CC2)Cc2ccc(C#N)cc2)cc1-c1ccc(CNC(C)c2ccc(C)cc2)o1] | ATAD2/ATAD2B |
| 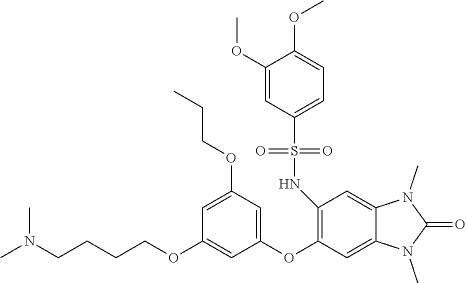 [SMILES: CCCOc1cc(OCCCCN(C)C)cc(Oc2cc3c(cc2NS(=O)(=O)c2ccc(OC)c(OC)c2)n(C)c(=O)n3C)c1] | TRIM24 |
| 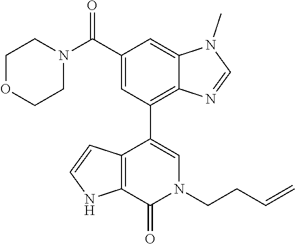 [SMILES: C=CCCn1cc(-c2cc(C(=O)N3CCOCC3)cc3c2ncn3C)c2cc[nH]c2c1=O] | TAF1 |

Oligomeric Backbone—Linker Moiety

The oligomeric backbone is a linker that connects the first terminus and the second terminus and brings the regulatory molecule in proximity to the target gene to modulate gene expression.

The length of the linker depends on the type of regulatory protein and also the target gene. In some embodiments, the linker has a length of less than about 50 Angstroms. In some embodiments, the linker has a length of about 20 to 30 Angstroms.

In some embodiments, the oligomeric backbone comprises between 5 and 50 chain atoms.

In some embodiments, the oligomeric backbone comprises a multimer having 2 to 50 spacing moieties, wherein each spacing moiety is independently selected from the group consisting of —((CR^{3a}R^{3b})ₓ—O)_{y}—, —((CR^{3a}R^{3b})ₓ—NR^{4a})_{y}—, —((CR^{3a}R^{3b})ₓ—CH═CH—(CR^{3a}R^{3b})ₓ—O)_{y}—, optionally substituted C₁-C₁₂ alkyl, optionally substituted C₂-C₁₀ alkenyl, optionally substituted C₂-C₁₀ alkynyl, optionally substituted C₆-C₁₀ arylene, optionally substituted C₃-C₇ cycloalkylene, optionally substituted 5- to 10-membered heteroarylene, optionally substituted 4- to 10-membered heterocycloalkylene, amino acid residue, —O—, —C(O)NR^{4a}—, —NR^{4a}C(O)—, —C(O)—, —NR^{1a}—, —C(O)O—, —S—, —S(O)—, —S(O)₂—, —S(O)₂NR^{4a}—, —NR^{4a}S(O)₂—, and —P(O)OH—, and any combinations thereof, wherein
each x is independently 2-4;
each y is independently 1-10;
each R^{1a} is independently a hydrogen or optionally substituted C₁-C₆ alkyl;
each R^{3a} and R^{3b} is independently selected from hydrogen, optionally substituted alkyl, optionally substituted alkenyl, optionally substituted alkynyl, optionally substituted alkoxy, optionally substituted amino, carboxyl, carboxyl ester, acyl, acyloxy, acyl amino, amino acyl, optionally substituted alkylamide, sulfonyl, optionally substituted thioalkoxy, optionally substituted aryl, optionally substituted heteroaryl, optionally substituted cycloalkyl, and optionally substituted heterocyclyl; and
each R^{4a} is independently a hydrogen or an optionally substituted C₁-C₆ alkyl.

In some embodiments, the oligomeric backbone comprises a multimer having 2 to 50 spacing moieties, wherein each spacing moiety is independently selected from the group consisting of optionally substituted C₁-C₁₂ alkyl, —((CH₂)ₓ—O)_{y}—, —((CH₂)ₓ—NH)_{y}—, —O—, —C(O)NH—, —NH—, and any combinations thereof.

In some embodiments, the oligomeric backbone comprises -(T¹-V¹)ₐ-(T²-V²)_{b}-(T³-V³)_{c}-(T⁴-V⁴)ₐ-(T⁵-V⁵)ₑ-, wherein
a, b, c, d and e are each independently O or 1, and where the sum of a, b, c, d and e is 1 to 5;
T¹, T², T³, T⁴ and T⁵ are each independently selected from an optionally substituted C₁-C₁₂ alkylene, optionally substituted alkenylene, optionally substituted alkynylene, (EA)_{w}, (EDA)ₘ, (PEG)ₙ, (modified PEG)ₙ, (AA)ₚ, —(CR^{2a}OH)ₕ—, optionally substituted C₆-C₁₀ arylene, optionally substituted C₃-C₇ cycloalkylene, optionally substituted 5- to 10-membered heteroarylene, optionally substituted 4- to 10-membered heterocycloalkylene, an acetal group, a disulfide, a hydrazine, a carbohydrate, a beta-lactam, and an ester,
(a) w is an integer from 1 to 20;
(b) m is an integer from 1 to 20;
(c) n is an integer from 1 to 30;
(d) p is an integer from 1 to 20;
(e) h is an integer from 1 to 12;
(f) EA has the following structure

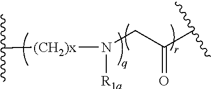

(g) EDA has the following structure:

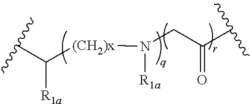

wherein each q is independently an integer from 1 to 6, each x is independently an integer from 1 to 4, and each r is independently 0 or 1;
(h) (PEG)ₙ has the structure of —(CR^{2a}R^{2b}—CR^{2a}R^{2b}—O)ₙ—CR^{2a}R^{2b}—;
(i) (modified PEG)ₙ has the structure of replacing at least one —(CR^{2a}R^{2b}—CR^{2a}R^{2b}—O)— in (PEG)ₙ with —(CH₂—CR^{2a}═CR^{2a}—CH₂—O)— or —(CR^{2a}R^{2b}—CR^{2a}R^{2b}—S)—;
(j) AA is an amino acid residue;
(k) V¹, V², V³, V⁴ and V⁵ are each independently selected from the group consisting of a bond, C(O)—, —NR^{1a}—, —C(O)NR^{1a}—, —NR^{1a}C(O)—, —CONR^{1a}—C₁-C₄ alkyl-, —NR^{1a}C(O)—C₁-C₄ alkyl-, —C(O)O—, —OC(O)—, —O—, —S—, —S(O)—, —S(O)₂—, —S(O)₂NR^{1a}—, —NR^{1a}S(O)₂— and —P(O)OH—;
(l) each Ria is independently hydrogen or and optionally substituted C₁-C₆ alkyl; and
   each R^{2a} and R^{2b} is independently selected from hydrogen, alkyl, substituted alkyl, alkenyl, substituted alkenyl, alkynyl, substituted alkynyl, halogen, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In some embodiments, the a, b, c, d and e are each independently 0 or 1, where the sum of a, b, c, d and e is 1. In some embodiments, the a, b, c, d and e are each independently 0 or 1, where the sum of a, b, c, d and e is 2. In some embodiments, the a, b, c, d and e are each independently 0 or 1, where the sum of a, b, c, d and e is 3. In some embodiments, the a, b, c, d and e are each independently 0 or 1, where the sum of a, b, c, d and e is 4. In some embodiments, the a, b, c, d and e are each independently 0 or 1, where the sum of a, b, c, d and e is 5.

In some embodiments, n is 3-9. In some embodiments, n is 4-8. In some embodiments, n is 5 or 6.

In some embodiments, T¹, T², T³, and T⁴, and T⁵ are each independently selected from C₁-C₁₂ alkyl, substituted C₁-C₁₂ alkyl, (EA)_{w}, (EDA)ₘ, (PEG)ₙ, (modified PEG)ₙ, (AA)ₚ, —(CR^{2a}OH)ₕ—, phenyl, substituted phenyl, piperidin-4-amino (P4A), para-amino-benzyloxycarbonyl (PABC), meta-amino-benzyloxycarbonyl (MABC), para-amino-benzyloxy (PABO), meta-amino-benzyloxy (MABO), para-aminobenzyl, an acetal group, a disulfide, a hydrazine, a carbohydrate, a beta-lactam, an ester, (AA)ₚ-MABC-(AA)ₚ, (AA)ₚ-MABO-(AA)ₚ, (AA)ₚ-PABO-(AA)ₚ and (AA)ₚ-PABC-(AA)ₚ. In some embodiments, piperidin-4-amino (P4A) is

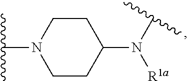

wherein R^{1a} is hydrogen or C₁-C₆ alkyl.

In some embodiments, T¹, T₂, T³, T⁴ and T⁵ are each independently selected from (C₁-C₁₂)alkyl, substituted C₁-C₁₂ alkyl, (EA)_{w}, (EDA)ₘ, (PEG)ₙ, (modified PEG)ₙ, (AA)ₚ, —(CR^{2a}OH)ₕ—, optionally substituted C₆-C₁₀ arylene, 4 to 10-membered heterocycloalkene, optionally substituted 5 to 10-membered heteroarylene. In some embodiments, EA has the following structure:

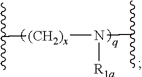

and
EDA has the following structure:

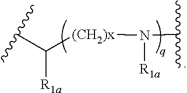

In some embodiments, x is 2-3 and q is 1-3 for EA and EDA. In some embodiments, Ria is hydrogen or C₁-C₆ alkyl.

In some embodiments, T⁴ or T⁵ is an optionally substituted C₆-C₁₀ arylene.

In some embodiments, T⁴ or T⁵ is phenylene or substituted phenylene. In some embodiments, T⁴ or T⁵ is phenylene or phenylene substituted with 1-3 substituents selected from C₁-C₆ alkyl, halogen, OH or amine. In some embodiments, T⁴ or T⁵ is 5 to 10-membered heteroarylene or substituted heteroarylene. In some embodiments, T⁴ or T⁵ is 4 to 10-membered heterocyclene or substituted heterocylcylene. In some embodiments, T⁴ or T⁵ is heteroarylene or heterocylene optionally substituted with 1-3 substituents selected from C₁-C₆ alkyl, halogen, OH or amine.

In some embodiments, T¹, T², T³, T⁴ and T⁵ and V¹, V², V³, V⁴ and V⁵ are selected from the following Table 3.

TABLE 3
Exemplary linker units.

| T¹ | V¹ | T² | V² | T³ | V³ | T⁴ | V⁴ | T⁵ | V⁵ |
|---|---|---|---|---|---|---|---|---|---|
| C₁-C₁₂ | C(O)NR^{1a} | (EA)_{w} | CO | (PEG)ₙ | NR^{1a}CO | — | — | — | — |
| alkylene | | | | | | | | | |
| C₁-C₁₂ | C(O)NR^{1a} | (EA)_{w} | CO | (PEG)ₙ | O | arylene | NR^{1a}CO | — | — |
| alkylene | | | | | | | | | |
| C₁-C₁₂ | C(O)NR^{1a} | (EA)_{w} | CO | (PEG)ₙ | O | Subst. | NR^{1a}CO | — | — |
| alkylene | | | | | | arylene | | | |
| C₁-C₁₂ | C(O)NR^{1a} | (EA)_{w} | CO | (PEG)ₙ | O | NR^{1a}CO | C₁-C₁₂ | Subst. | NR^{1a}CO |
| alkylene | | | | | | | alkyl | arylene | |
| C₁-C₁₂ | C(O)NR^{1a} | (EA)_{w} | CO | C₁-C₁₂ | NR^{1a}CO—C₁- | Subst. | NR¹¹ | — | — |
| alkylene | | | | alkyl | C₄ | arylene | | | |
| | | | | | alkyl | | | | |
| C₁-C₁₂ | C(O)NR^{1a} | (EA)_{w} | CO | (PEG)ₙ | O | Subst. | — | — | — |
| alkylene | | | | | | arylene | | | |
| (PEG)ₙ | C(O)NR^{1a}—C₁- | — | — | — | — | — | — | — | — |
| | C₄ | | | | | | | | |
| | alkyl | | | | | | | | |
| (EA)_{w} | CO | C₁-C₁₂ | C(O)NR^{1a}—C₁- | — | — | — | — | — | — |
| | | alkyl | C₄ | | | | | | |
| | | | alkyl | | | | | | |
| C₁-C₁₂ | C(O)NR^{1a} | (EA)_{w} | CO | (PEG)ₙ | NR^{1a}CO—C₁- | — | — | — | — |
| alkylene | | | | | C₄ | | | | |
| | | | | | alkyl | | | | |
| (EA)_{w} | CO | (PEG)ₙ | O | phenyl | NR^{1a}CO—C₁- | — | — | — | — |
| | | | | | C₄ | | | | |
| | | | | | alkyl | | | | |
| C₁-C₁₂ | C(O)NR^{1a} | (PEG)ₙ | CO | — | — | — | — | — | — |
| alkylene | | | | | | | | | |
| C₁-C₁₂ | C(O)NR^{1a} | (EA)_{w} | CO | modifd. | O | arylene | NR^{1a}CO | — | — |
| alkylene | | | | (PEG)ₙ | | | | | |

In some embodiments, the oligomeric backbone comprises N(R^{1a})(CH₂)ₓN(R^{1b})(CH₂)ₓN—, wherein R^{1a} and R^{1b} are each independently selected from hydrogen or optionally substituted C₁-C₆ alkyl; and each x is independently an integer in the range of 1-6.

In some embodiments, the oligomeric backbone comprises —(CH₂—C(O)N(R^{4a})—(CH₂)_{q}—N(R^{4a})—(CH₂)_{q}—N(R^{4a})C(O)—(CH₂)ₓ—C(O)N(R^{4a})-A-, —(CH₂)ₓ—C(O)N(R^{4a})—(CH₂CH₂O)_{y}(CH₂)ₓ—C(O)N(R^{4a})-A-, or —C(O)N(R^{4a})—(CH₂)_{q}—N(R^{4a})—(CH₂)_{q}—N(R^{4a})C(O)—(CH₂)ₓ-A-; wherein each q is independently an integer from 2 to 10; each x is independently an integer from 1-6; and each A is independently selected from a bond, an optionally substituted C₁-C₁₂ alkyl, an optionally substituted C₆-C₁₀ arylene, optionally substituted C₃-C₇ cycloalkylene, optionally substituted 5 to 10-membered heteroarylene, and optionally substituted 4 to 10-membered heterocycloalkylene.

In some embodiments, the oligomeric backbone comprises —(CH₂CH₂—O)ₓ₁— or —(CH₂CH₂—O)ₓ₂-A-(CH₂CH₂—O)ₓ₃—, wherein A is an optionally substituted 4 to 10-membered heterocycloalkylene or spirocyclene, and each x₁, x₂, and x₃ is independently an integer from 1-15.

In some embodiments, the oligomeric backbone comprises —NR^{4a}—(CH₂CH₂O)_{y}(CH₂)ₓ— or —NR^{4a}—(CH₂)_{q}—C(O)NR^{4a}(CH₂CH₂O)_{y}(CH₂)ₓ—, wherein q is 2-10, x is 1-4, y is 1-50, and each R^{4a} is independently hydrogen or an optionally substituted C₁-C₆ alkyl. In some embodiments, the oligomeric backbone comprises —NR^{4a}—(CH₂CH₂O)_{y}(CH₂)ₓ. In some embodiments, the oligomeric backbone comprises —NR^{4a}—(CH₂)_{q}—C(O)NR^{4a}(CH₂CH₂O)_{y}(CH₂)ₓ—.

In some embodiments, the oligomeric backbone comprises —(CH₂CH₂—O)ₓ₁—, —(CH₂CH₂—O)ₓ₁—(CH₂CH₂)—NH—, —NH—(CH₂CH₂—O)ₓ₁—, —NH—(CH₂CH₂—O)ₓ₁—(CH₂CH₂)—NH—, —(CH₂CH₂—O)ₓ₁—(CH₂CH₂)—NHC(O)—, or —NH—(CH₂CH₂—O)ₓ₁—(CH₂CH₂)—NHC(O)—. In some embodiments, the oligomeric backbone comprises —NH—(CH₂CH₂—O)ₓ₁— or —NH—(CH₂CH₂—O)ₓ₁—(CH₂CH₂)—NH—. In some embodiments, the oligomeric backbone comprises —NH—(CH₂CH₂—O)ₓ₁—. In some embodiments, the oligomeric backbone comprises —NH—(CH₂CH₂—O)ₓ₁—(CH₂CH₂)—NH—.

In some embodiments, the oligomeric backbone comprises polyethylene glycol (PEG). In some embodiments, the oligomeric backbone comprises 1-20 PEG units. In some embodiments, the oligomeric backbone comprises 1, 2, 3, 4, 5, 6, 7, 8, 9, 10, 11, 12, 13, 14, 15, 16, 17, 18, 19, or 20 PEG units.

In some embodiments, A is selected from

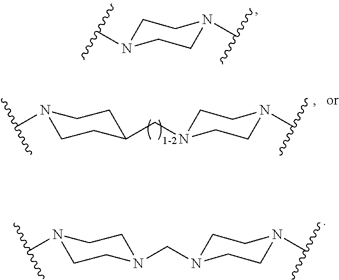

In some embodiments, A is

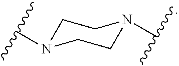 [SMILES: *N1CCN(*)CC1]

In some embodiments, A is

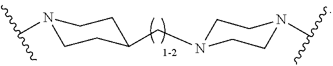

In some embodiments, A is

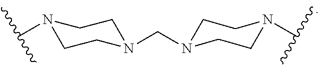 [SMILES: *N1CCN(CN2CCN(*)CC2)CC1]

In some embodiments, A comprises a moiety having the structure:

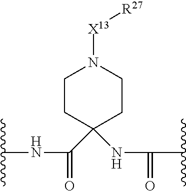

or a pharmaceutically acceptable salt thereof, wherein:
X¹³ is absent or —C(O)—; and
R²⁷ is an optionally substituted C₁-C₅₀ alkyl or optionally substituted C₁-C₅₀ heteroalkyl.

In some embodiments, X¹³ is —C(O)—. In some embodiments, X¹³ is absent.

In some embodiments, R²⁷ is C₁-C₅₀ alkyl. In some embodiments, R²⁷ is C₁-C₄₀ alkyl. In some embodiments, R²⁷ is C₁-C₃₀ alkyl. In some embodiments, R²⁷ is C₁-C₂₀ alkyl. In some embodiments, R²⁷ is C₁-C₁₀ alkyl. In some embodiments, R²⁷ is C₁-C₅₀ heteroalkyl. In some embodiments, R²⁶ is C₁-C₄₀ heteroalkyl. In some embodiments, R²⁷ is C₁-C₃₀ heteroalkyl. In some embodiments, R²⁷ is C₁-C₂₀ heteroalkyl. In some embodiments, R²⁷ is C₁-C₁₀ heteroalkyl. In some embodiments, the heteroalkyl is polyethylene glycol (PEG).

In some embodiments, the oligomeric backbone comprises a moiety having a structure of Formula (C-1):

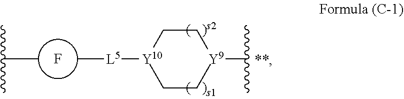

or a pharmaceutically acceptable salt thereof, wherein:
Ring F is absent, arylene, or heterocycloalkylene;
L⁵ is absent, optionally substituted alkylene, or optionally substituted alkynylene;
Y⁹ and Y¹⁰ are each independently CH or N;
s₁ and s₂ are each independently 0-3; and
** denotes attachment to the second terminus.

In some embodiments, Ring F is absent. In some embodiments, Ring F is C₄-C₇ heterocycloalkylene.

In some embodiments, Y⁹ is N. In some embodiments, Y⁹ is CH.

In some embodiments, Y¹⁰ is N. In some embodiments, Y¹⁰ is CH.

In some embodiments, L⁵ is absent.

In some embodiments, L⁵ is alkylene or alkynylene.

In some embodiments, L⁵ is —(CR^{1G}R^{1G})ₓ-(alkylene)₂-(CR^{1G}R^{1G})_{y}—; wherein x and y are each independently 0 or 1; and each R^{1G} is hydrogen or C₁-C₃ alkyl.

In some embodiments, the oligomeric backbone comprises a moiety having a structure of Formula (C-2):

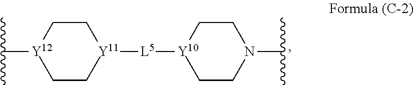

or a pharmaceutically acceptable salt thereof, wherein:
Y¹⁰, Y¹¹ and Y¹² are each independently N or CH.

In some embodiments, each of Y¹¹ and Y¹² is independently N or CH; and Y¹⁰ is N.

In some embodiments, L⁵ is C₁-C₃ alkylene or C₁-C₃ alkynylene. In some embodiments, L⁵ is C₁-C₃ alkylene. In some embodiments, L⁵ is C₁-C₃ alkynylene. In some embodiments, L⁵ is —CH₂—, —CH₂CH₂—, —C≡C—, or —C≡C—C≡C—. In some embodiments, L⁵ is —CH₂— or —CH₂CH₂—. In some embodiments, L⁵ is —C≡C—. In some embodiments, L⁵ is —C≡C—C≡C—.

In some embodiments, the oligomeric backbone comprises a moiety having the structure of Formula (C-3):

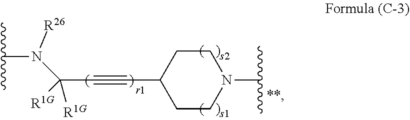

or a pharmaceutically acceptable salt thereof, wherein:
s₁ and s₂ are each independently 0-3;
r₁ is an integer from 1-3;
R²⁶ is an optionally substituted C₁-C₂₀ alkylene or optionally substituted C₂-C₂₀ heteroalkylene;
each R^{1G} is independently hydrogen or C₁-C₃ alkyl; and
** denotes attachment to the second terminus.

In some embodiments, R²⁶ is an optionally substituted C₁-C₂₀ heteroalkylene. In some embodiments, R²⁶ is PEG.

In some embodiments, each R^{1G} is independently hydrogen. In some embodiments, R^{1G} is independently C₁-C₃ alkyl. In some embodiments, the C₁-C₃ alkyl is methyl, ethyl or propyl. In some embodiments, each R^{1G} is independently methyl.

In some embodiments, s₁ and s₂ are each independently is 0, 1, or 2. In some embodiments, s₁ and s₂ are each independently 0. In some embodiments, s₁ and s₂ are each independently 1.

In some embodiments, r₁ is 1 or 2. In some embodiments, r₁ is 1. In some embodiments, r₁ is 2.

In some embodiments, the oligomeric backbone comprises:

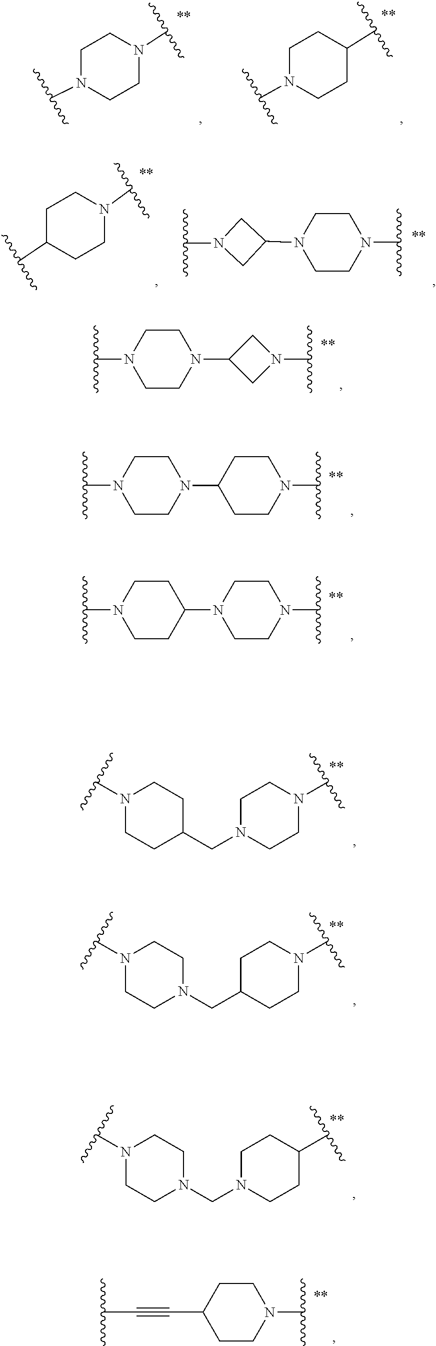

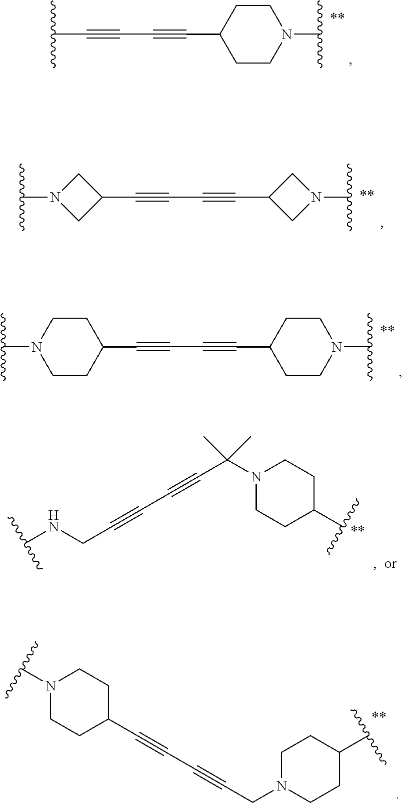 [SMILES: *C#CC#CC1CCN(*)CC1 ; *N1CC(C#CC#CC2CN(*)C2)C1 ; *N1CCC(C#CC#CC2CCN(*)CC2)CC1 ; *NCC#CC#CC(C)(C)N1CCC(*)CC1 ; *C1CCN(CC#CC#CC2CCN(*)CC2)CC1]

In some embodiments, the oligomeric backbone is joined with the first terminus and/or with the second terminus with a group selected from —C(O)—, —NR^{1a}—, —C(O)NR^{1a}—, —NR^{1a}C(O)—, —C(O)NR^{1a}C₁-C₄alkyl-, —NR^{1a}C(O)—C₁-C₄alkyl-, —C(O)O—, —OC(O)—, —O—, —S—, —S(O)—, —S(O)₂—, —S(O)₂NR^{1a}—, —NR^{1a}S(O)₂—, —P(O)OH—, —((CH₂)ₓ—O)—, —((CH₂)_{y}—NR^{1a})—, optionally substituted C₁-C₁₂ alkylene, optionally substituted C₂-C₁₀ alkenylene, optionally substituted C₂-C₁₀ alkynylene, optionally substituted C₆-C₁₀ arylene, optionally substituted C₃-C₇ cycloalkylene, optionally substituted 5 to 10-membered heteroarylene, and optionally substituted 4 to 10-membered heterocycloalkylene, wherein each x is independently 1-4, each y is independently 1-4, and each R^{1a} is independently a hydrogen or optionally substituted C₁-C₆ alkyl.

In some embodiments, the oligomeric backbone is joined with the first terminus with a group selected from —O—, —C(O)—, —NR^{1a}—, C₁-C₁₂ alkyl, —C(O)NR^{1a}—, and —NR^{1a}C(O)—. In some embodiments, the oligomeric backbone is joined with the first terminus with a group selected from —O— or —NR^{1a}—.

In some embodiments, the oligomeric backbone is joined with the second terminus with a group selected from —C(O)—, —NR^{1a}—, —C(O)NR^{1a}—, —NR^{1a}C(O)—, —((CH₂)ₓ—O)—, —((CH₂)_{y}—NR^{1a})—, —O—, optionally substituted C₁-C₁₂ alkyl, optionally substituted C₆-C₁₀ arylene, optionally substituted C₃-C₇ cycloalkylene, optionally substituted 5- to 10-membered heteroarylene, and optionally substituted 4- to 10-membered heterocycloalkylene, wherein each x is independently 1-4, each y is independently 1-4, and each R^{1a} is independently a hydrogen or optionally substituted C₁-C₆ alkyl.

In some embodiments, the oligomeric backbone is joined with the second terminus with a group selected from —O—, —C(O)—, —NR^{1a}—, C₁-C₁₂ alkyl, —C(O)NR^{1a}—, and —NR^{1a}C(O)—. In some embodiments, the oligomeric backbone is joined with the second terminus with a group selected from —O— or —NR^{1a}—. In some embodiments, the oligomeric backbone is joined with the second terminus with —O—. In some embodiments, the oligomeric backbone is joined with the second terminus with —NR^{1a}—. In some embodiments, the oligomeric backbone is joined with the second terminus with —NH—.

In some embodiments, non-limiting examples of the transcription modulator compounds described herein are presented below in Table 4 (next page).

TABLE 4
Compounds of the disclosure.

| Cmpd. | |
|---|---|
| No. | Structure |
| A-1 | 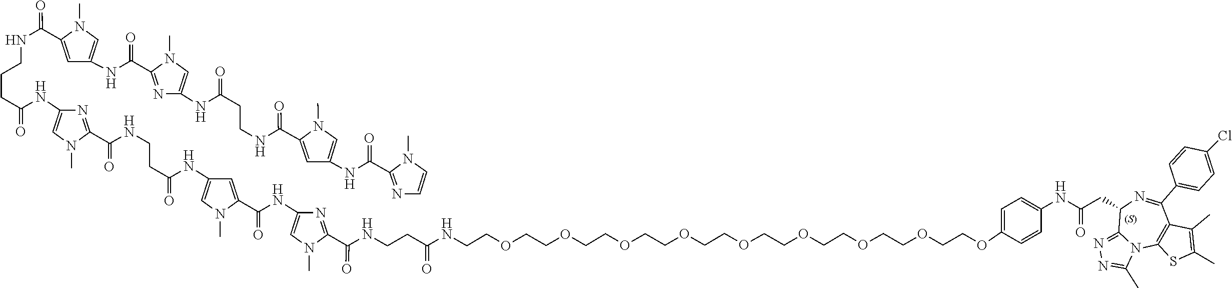 |
| A-2 | 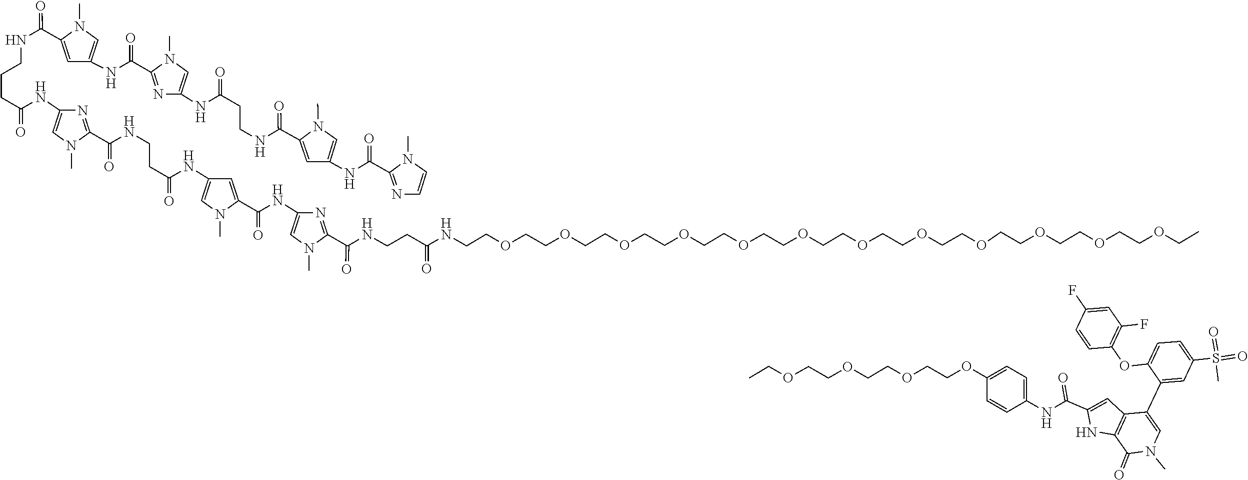 |
| A-3 | 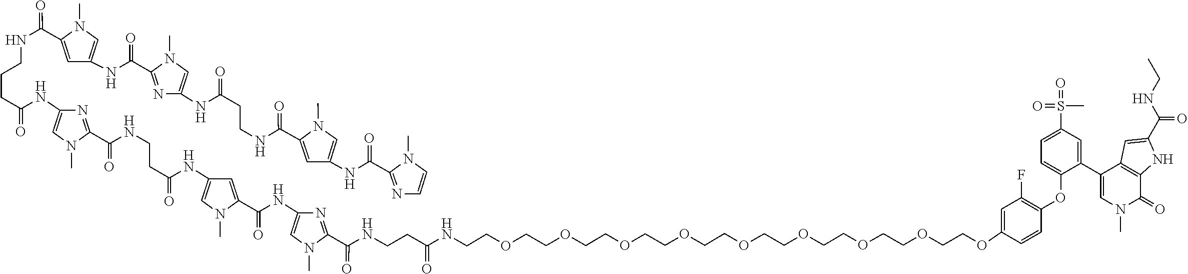 [SMILES: CCNC(=O)c1cc2c(-c3cc(S(C)(=O)=O)ccc3Oc3ccc(OCCOCCOCCOCCOCCOCCOCCOCCOCCNC(=O)CCNC(=O)c4nc(NC(=O)c5cc(NC(=O)CCNC(=O)c6nc(NC(=O)CCCNC(=O)c7cc(NC(=O)c8nc(NC(=O)CCNC(=O)c9cc(NC(=O)c%10nccn%10C)cn9C)cn8C)cn7C)cn6C)cn5C)cn4C)cc3F)cn(C)c(=O)c2[nH]1] |
| A-4 | 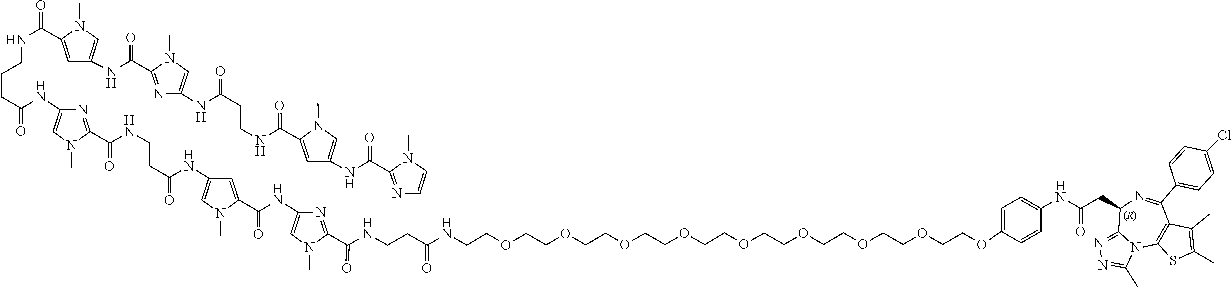 |
| A-5 | 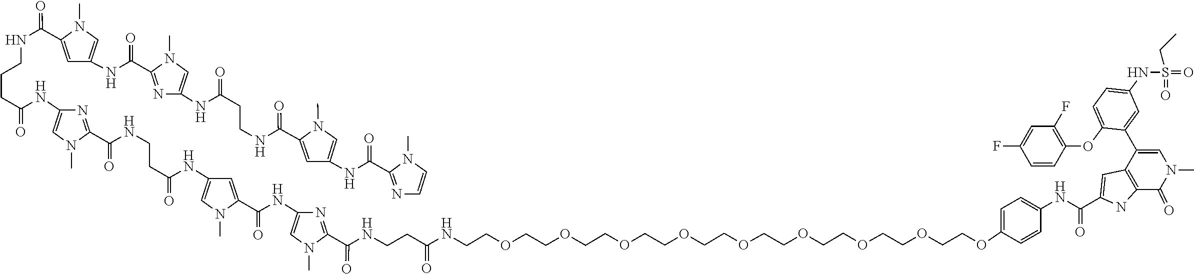 |
| A-6 | 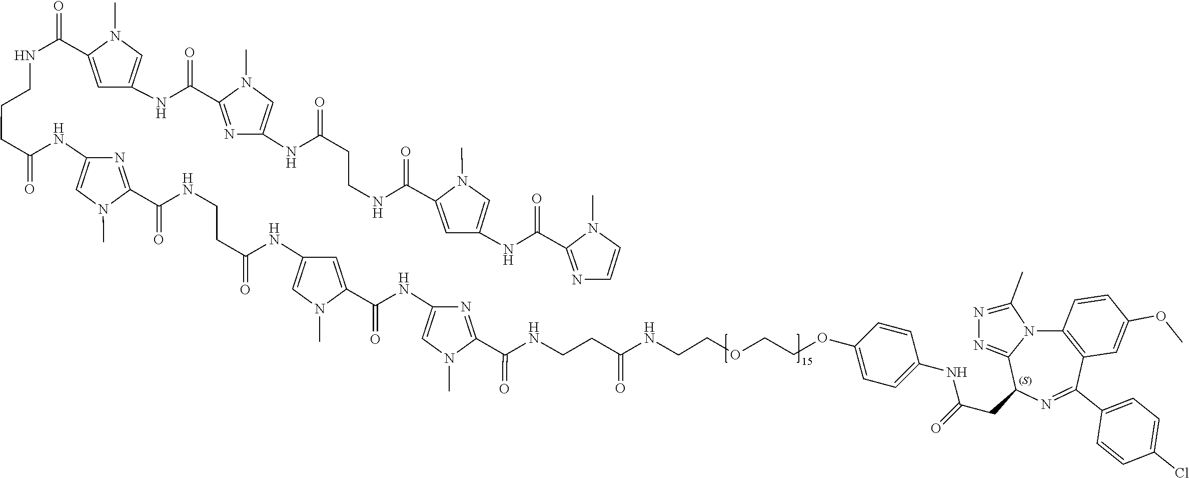 |
| A-7 | 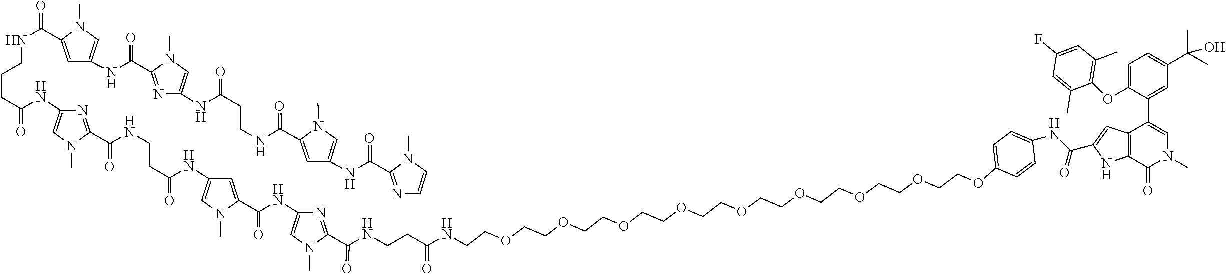 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCOCCNC(=O)CCNC(=O)c4nc(NC(=O)c5cc(NC(=O)CCNC(=O)c6nc(NC(=O)CCCNC(=O)c7cc(NC(=O)c8nc(NC(=O)CCNC(=O)c9cc(NC(=O)c%10nccn%10C)cn9C)cn8C)cn7C)cn6C)cn5C)cn4C)cc3)cc12] |
| A-8 | 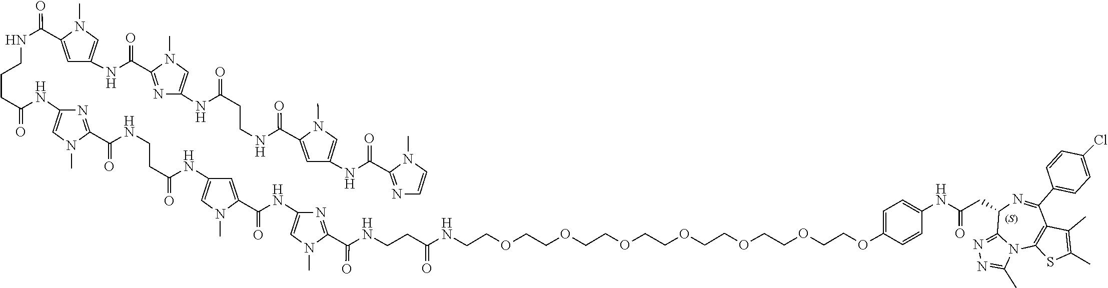 |
| A-9 | 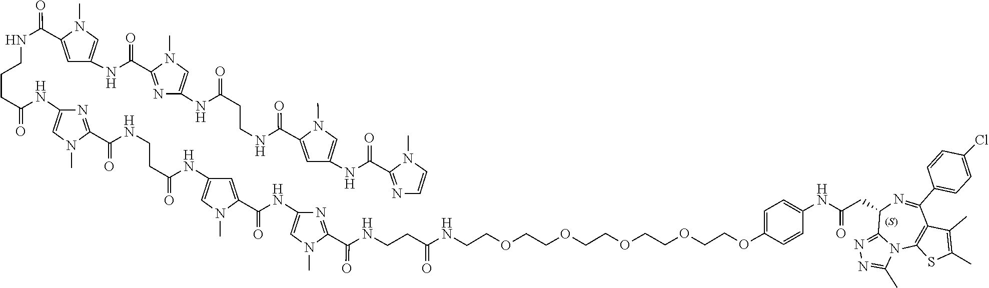 |
| A-10 | 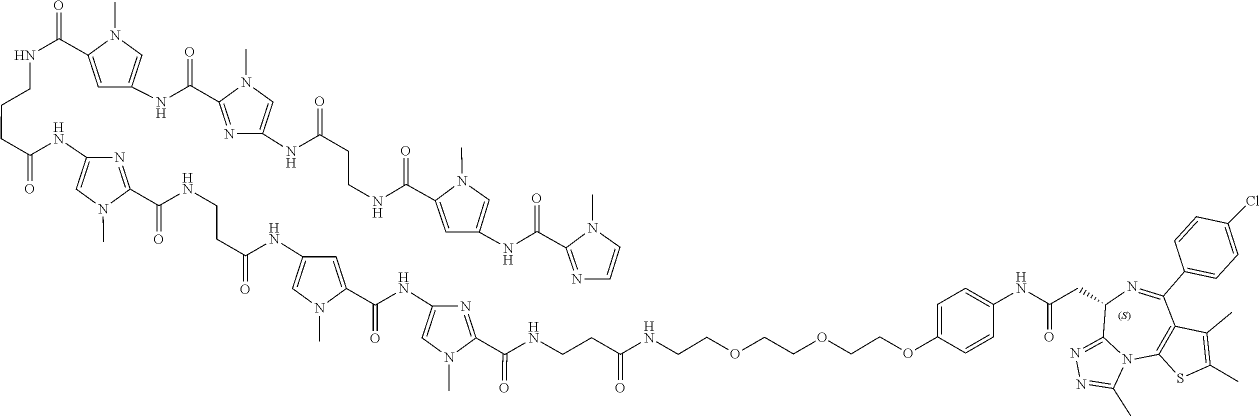 |
| A-11 | 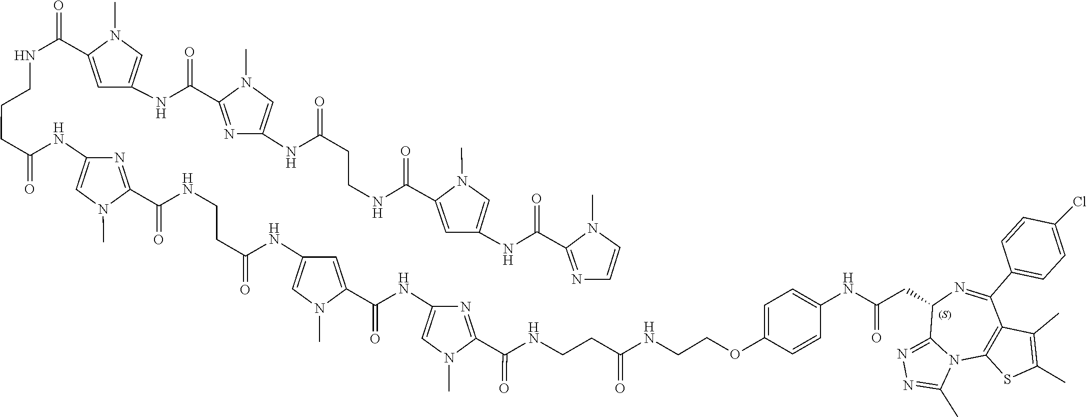 |
| A-12 | 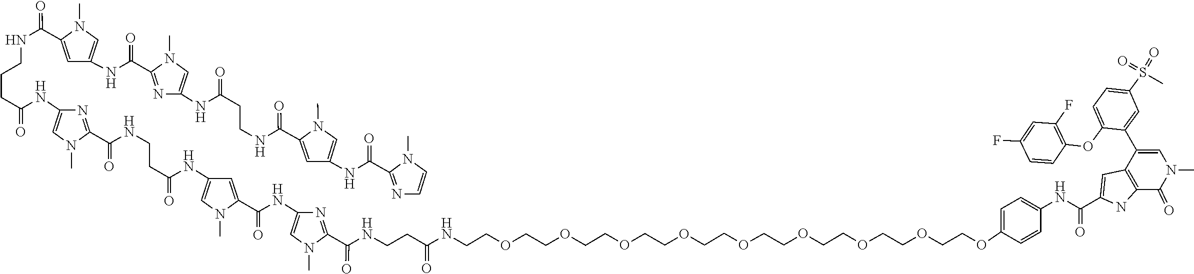 |
| A-13 | 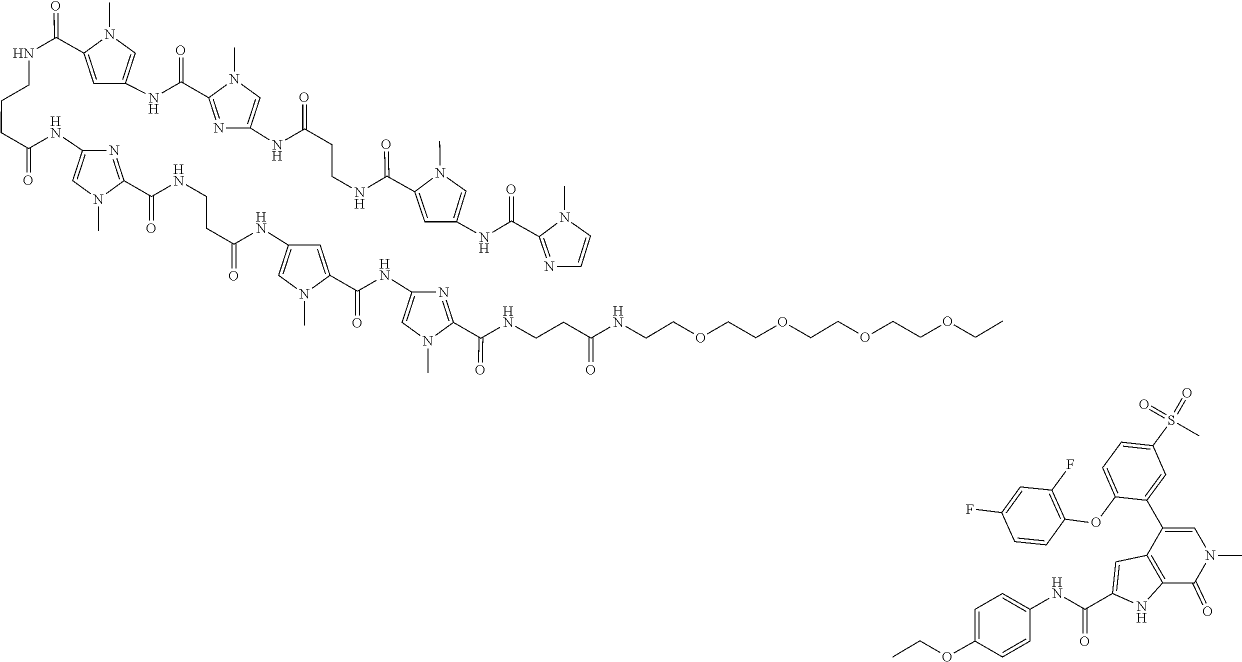 |
| A-14 | 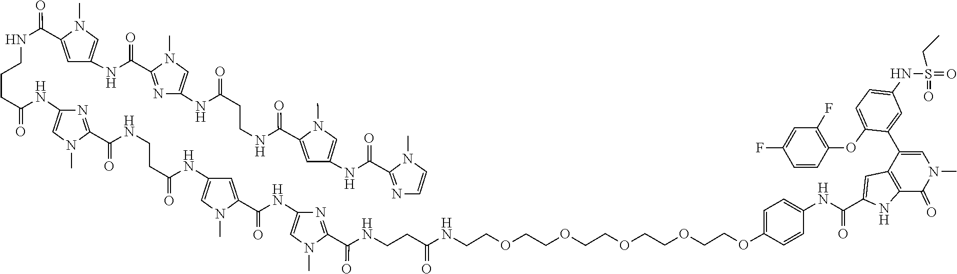 [SMILES: CCS(=O)(=O)Nc1ccc(Oc2ccc(F)cc2F)c(-c2cn(C)c(=O)c3[nH]c(C(=O)Nc4ccc(OCCOCCOCCOCCOCCNC(=O)CCNC(=O)c5nc(NC(=O)c6cc(NC(=O)CCNC(=O)c7nc(NC(=O)CCCNC(=O)c8cc(NC(=O)c9nc(NC(=O)CCNC(=O)c%10cc(NC(=O)c%11nccn%11C)cn%10C)cn9C)cn8C)cn7C)cn6C)cn5C)cc4)cc23)c1] |
| A-15 | 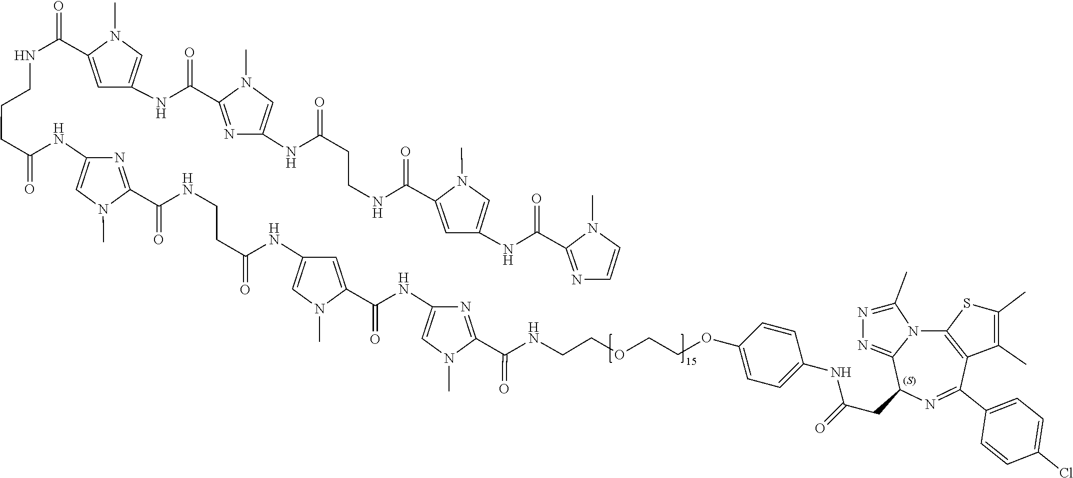 |
| A-16 | 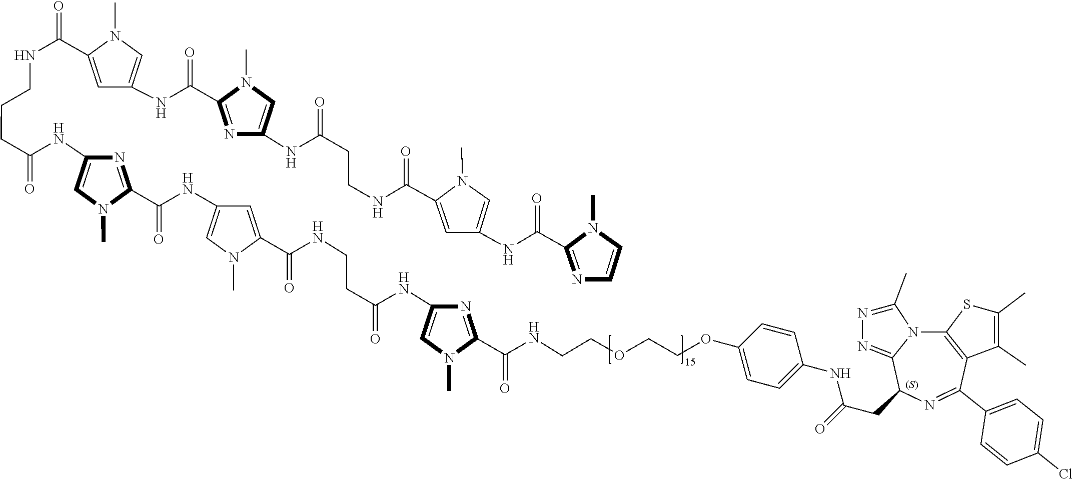 |
| A-17 | 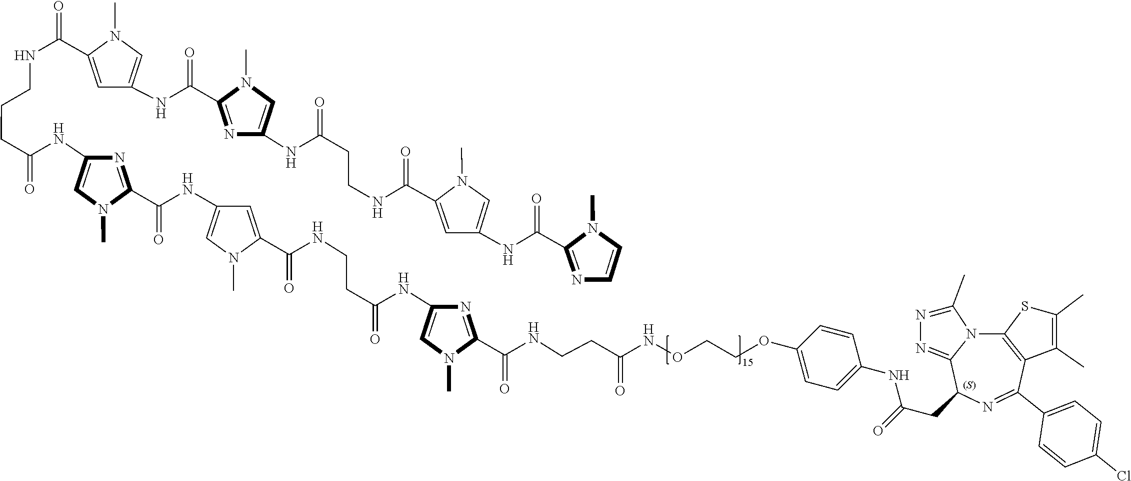 |
| A-18 | 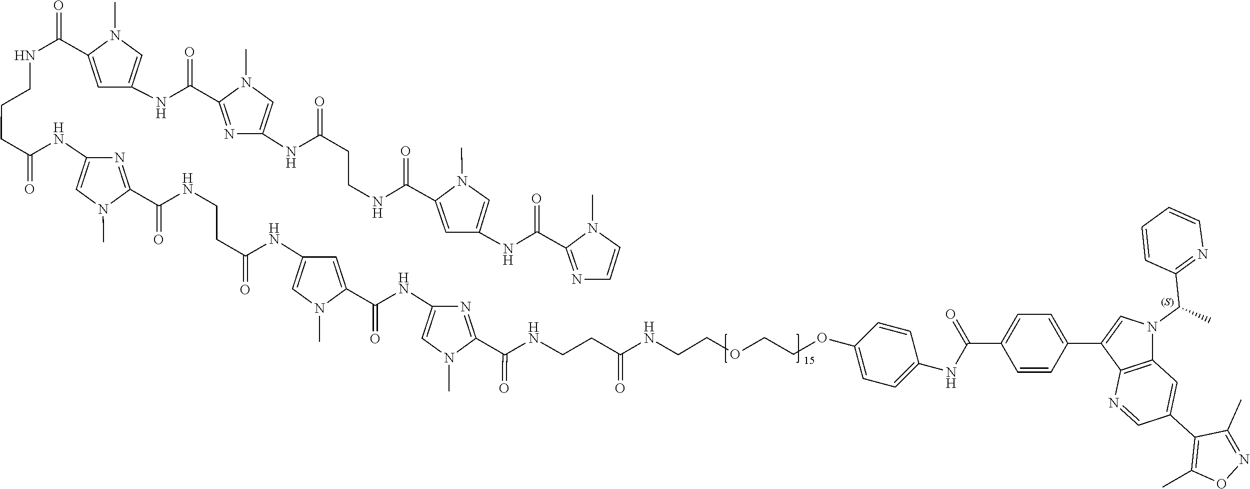 |
| A-19 | 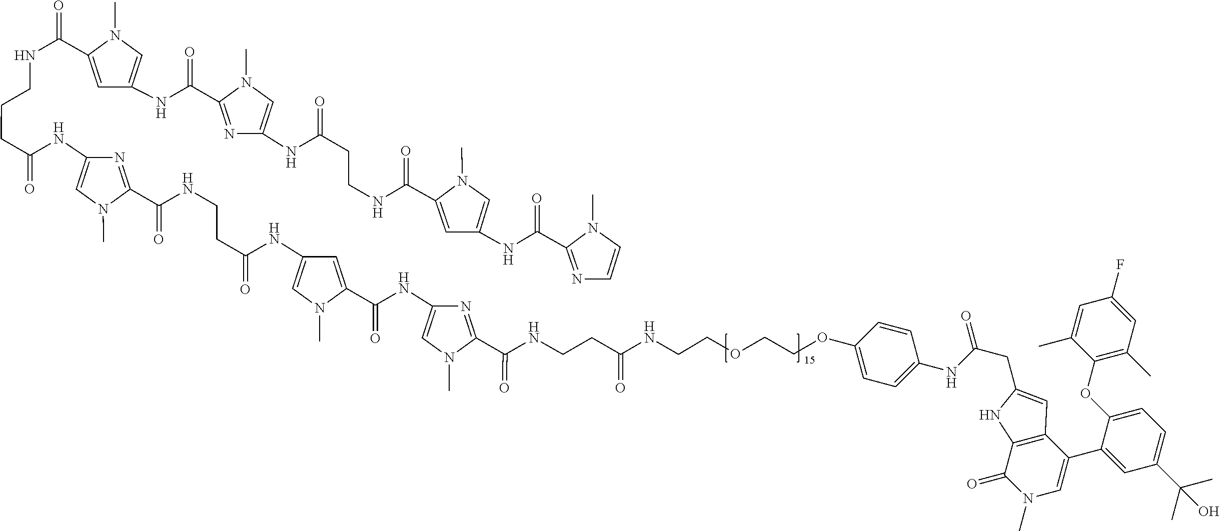 |
| A-20 | 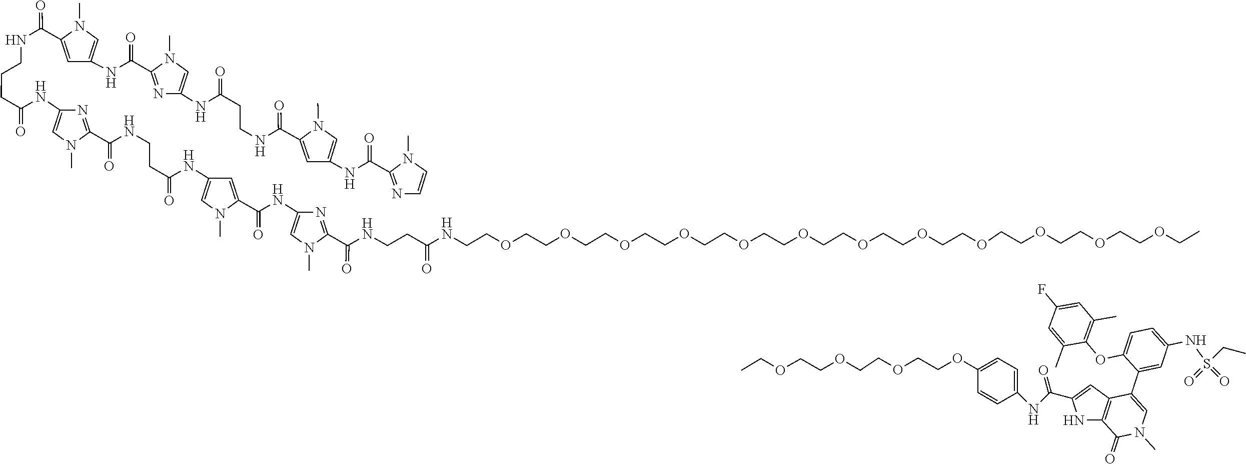 |
| A-21 | 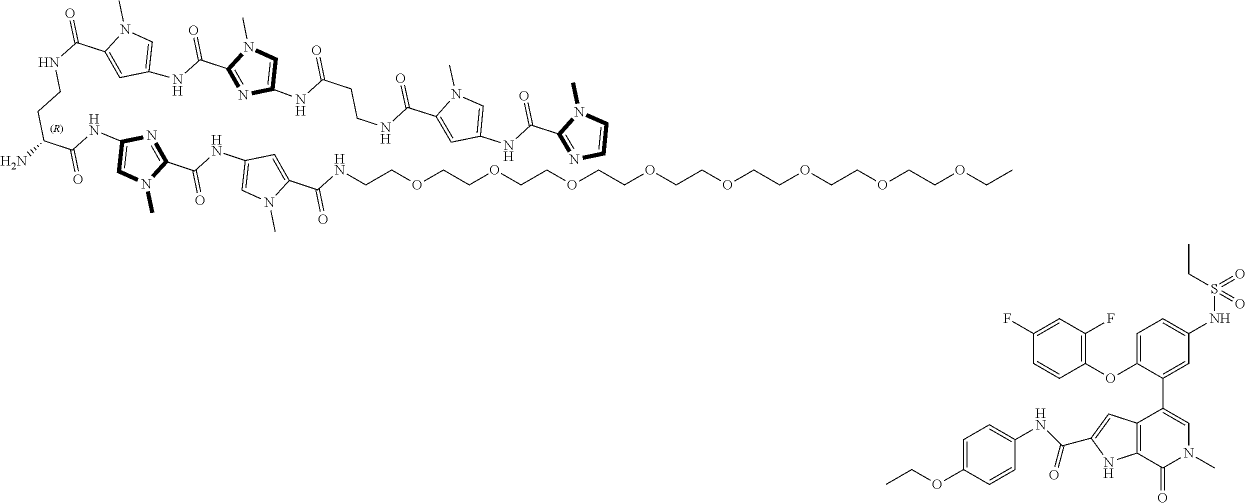 |
| A-22 | 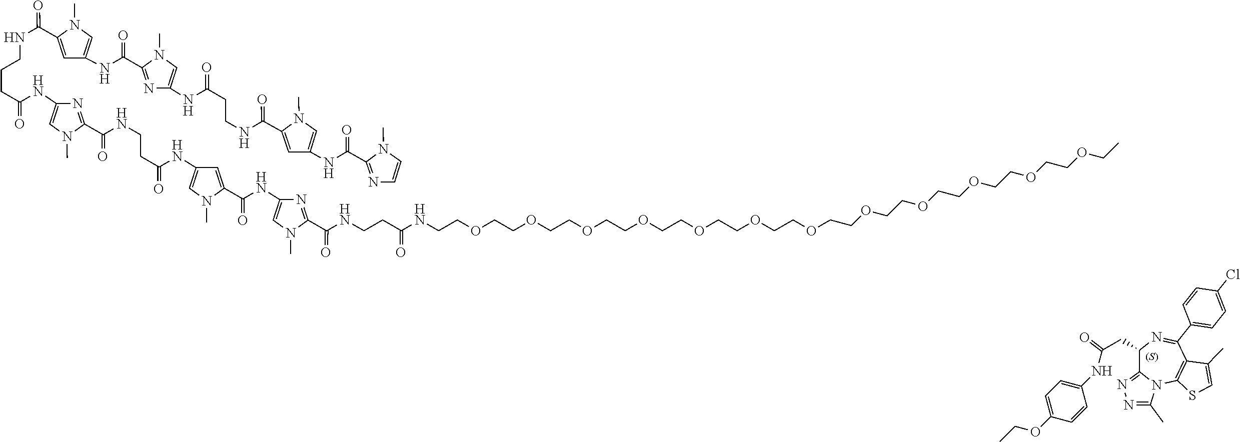 |
| A-23 | 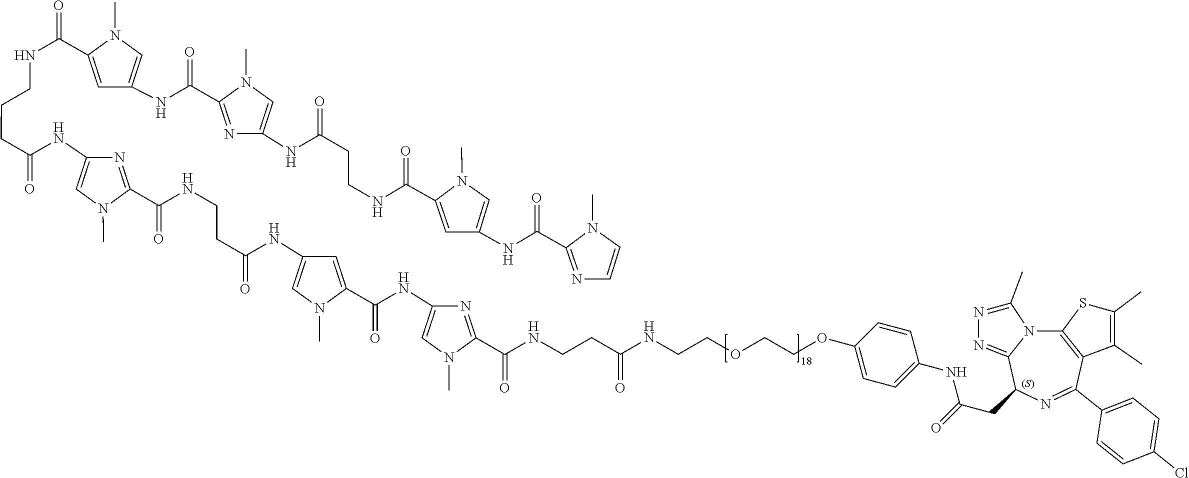 |
| A-24 | 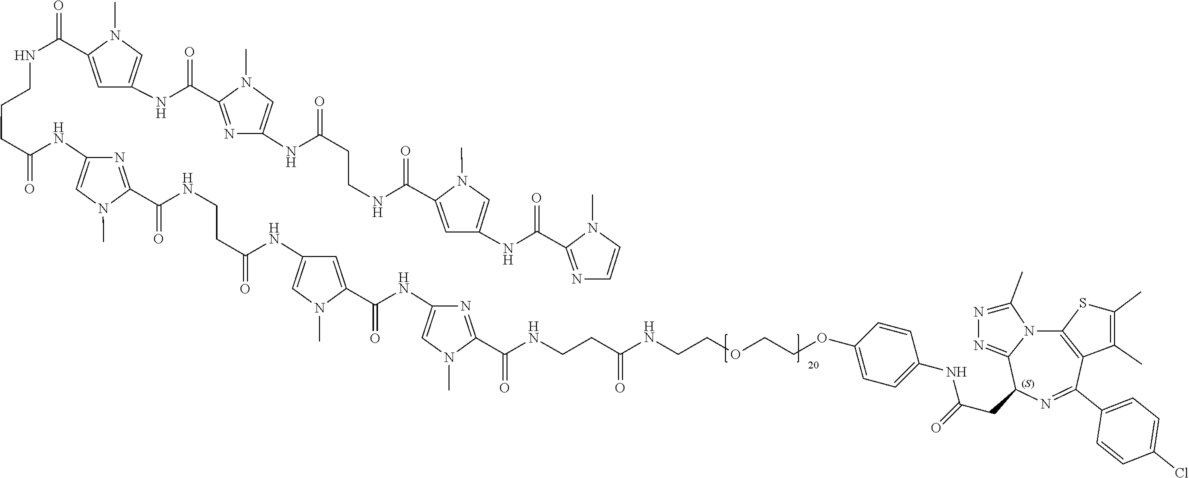 |
| A-25 | 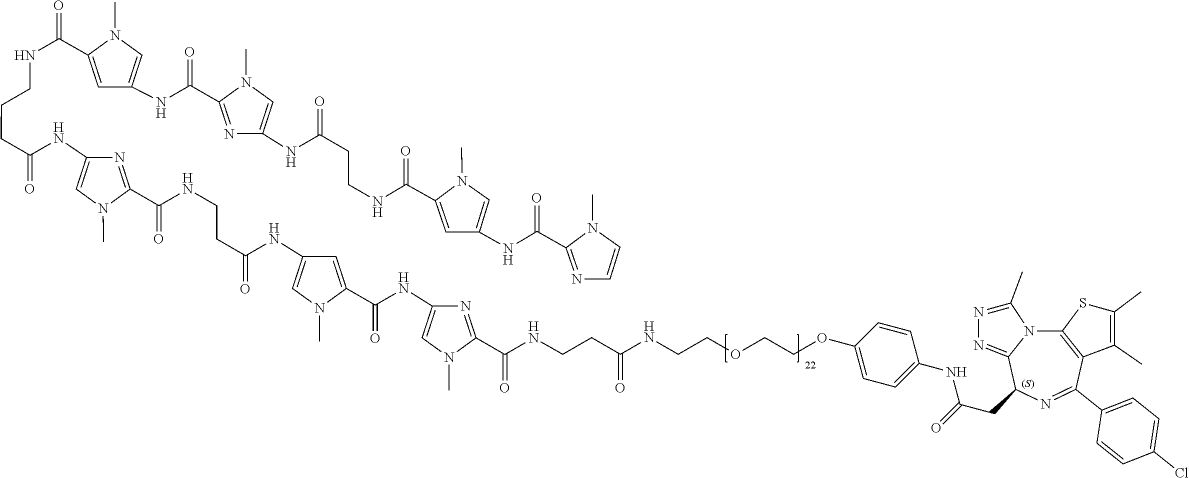 |
| A-26 | 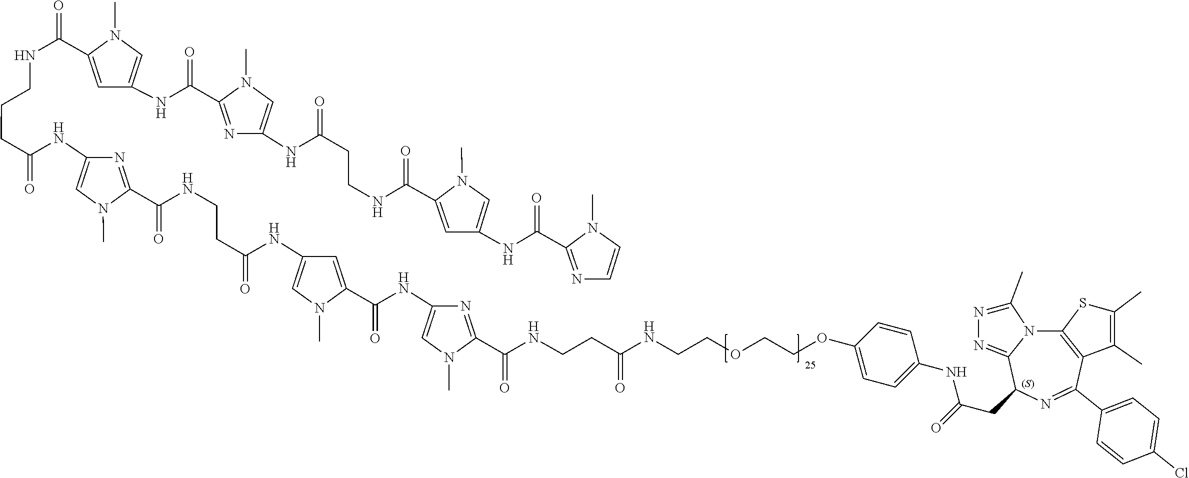 |
| A-27 | 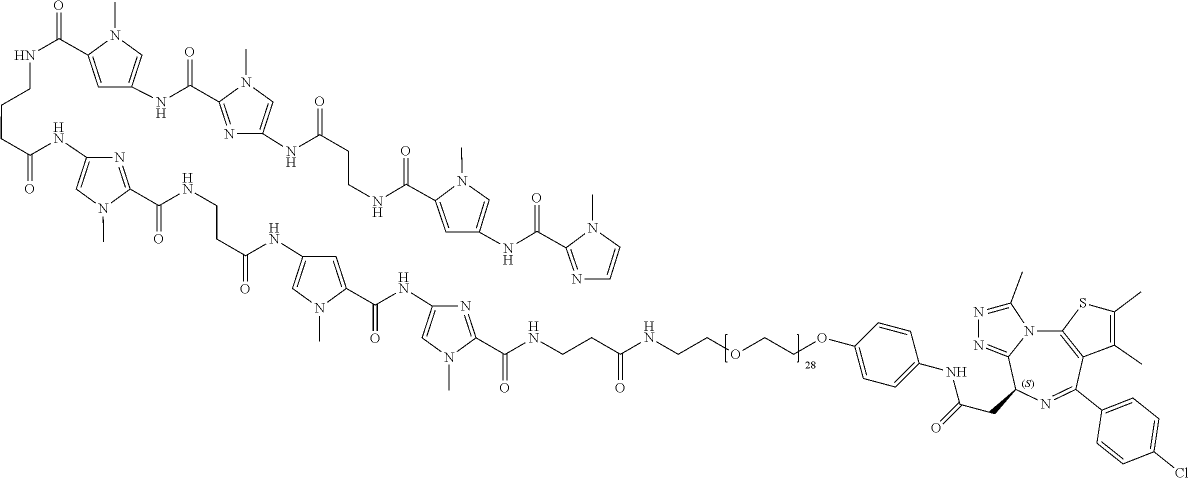 |
| A-28 | 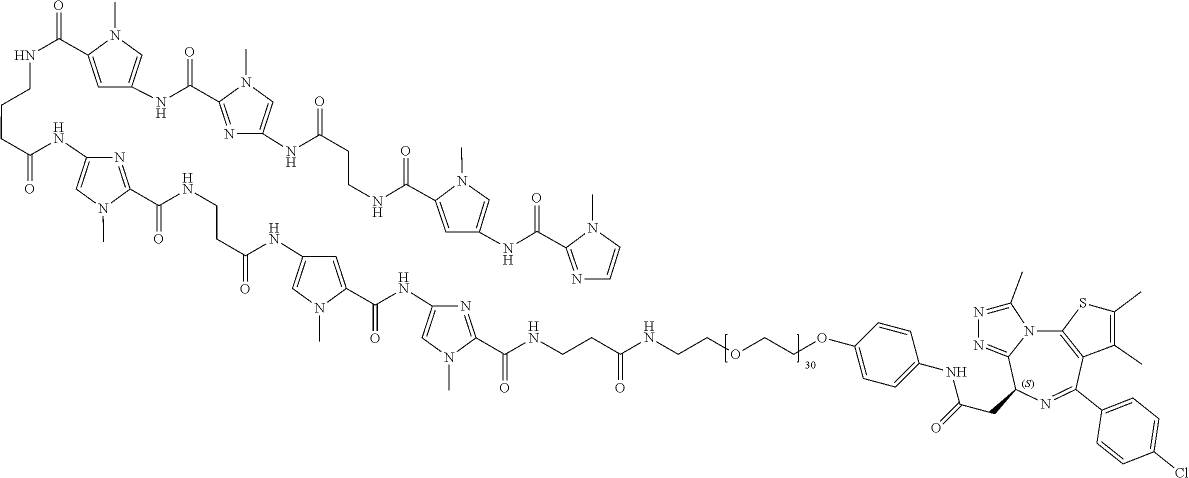 |
| A-29 | 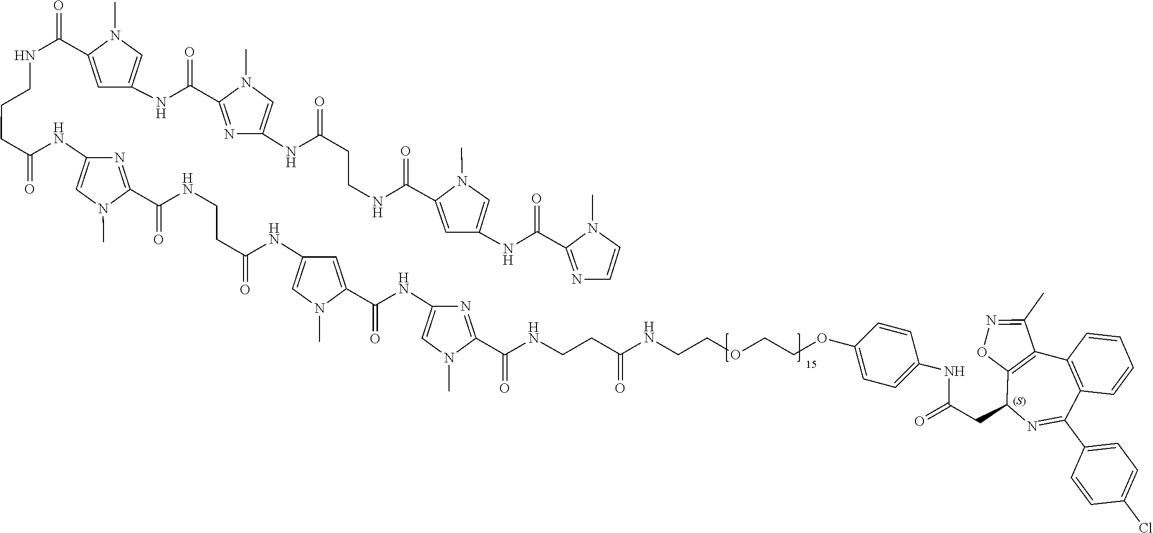 |
| A-30 | 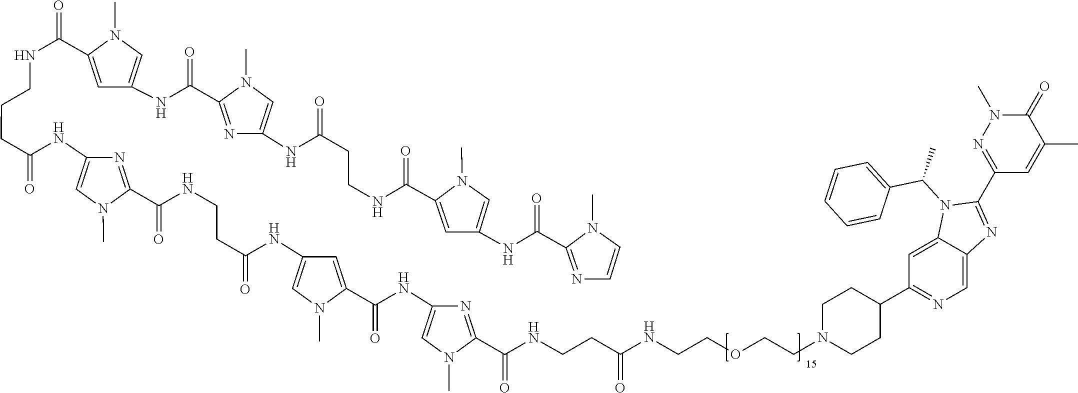 |
| A-31 | 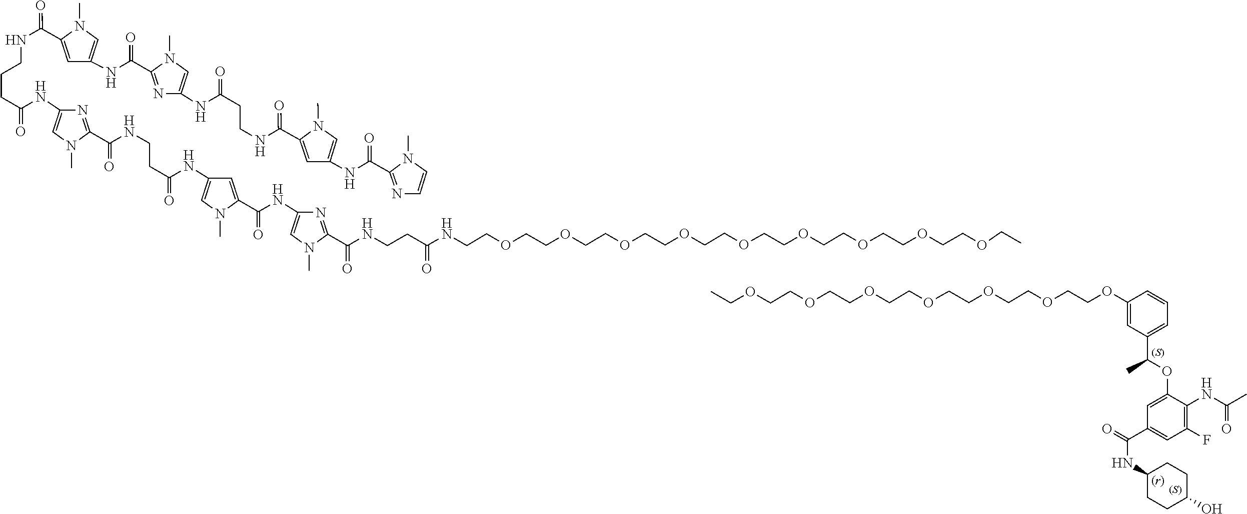 |
| A-32 | 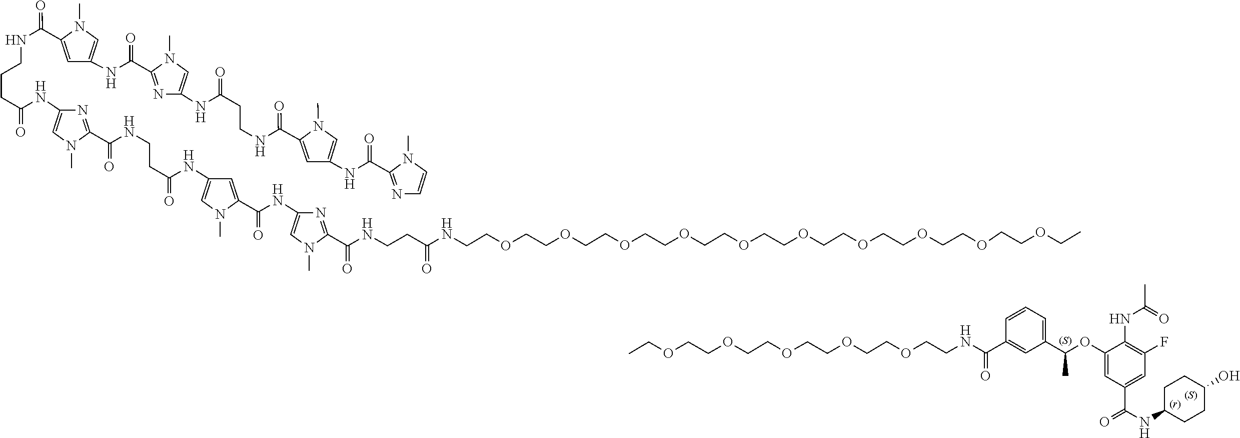 |
| A-33 | 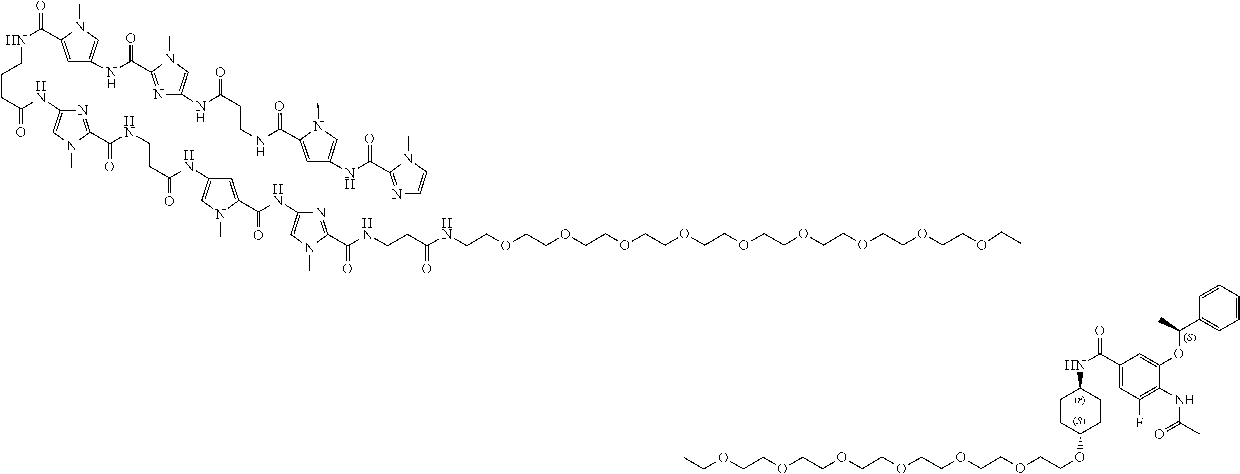 |
| A-34 | 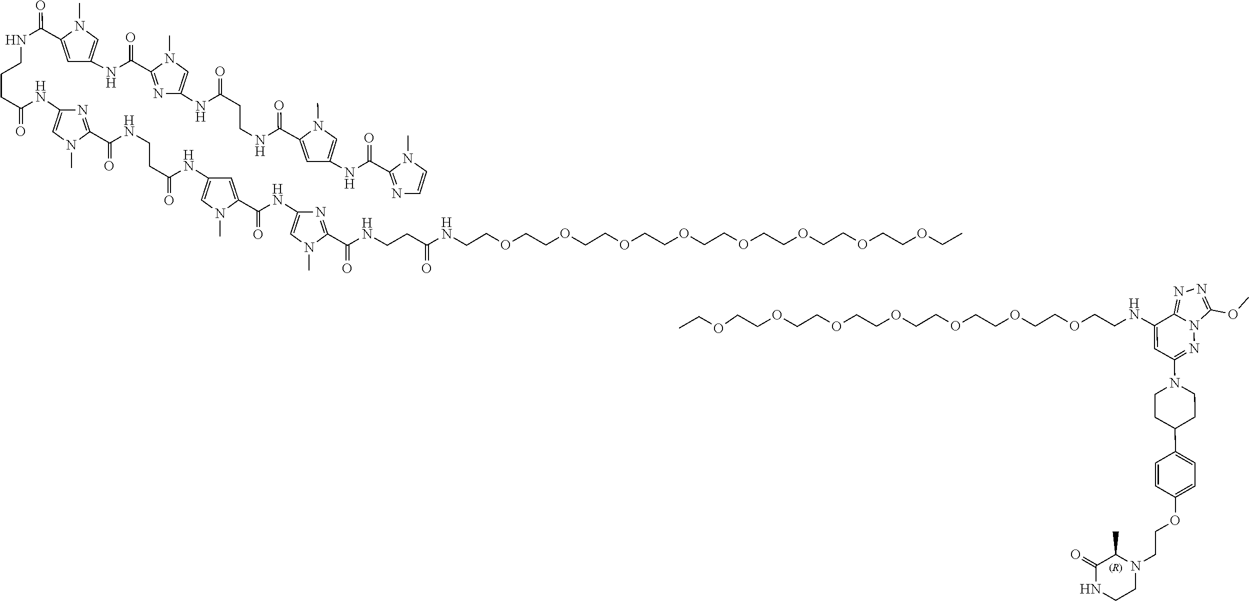 |
| A-35 | 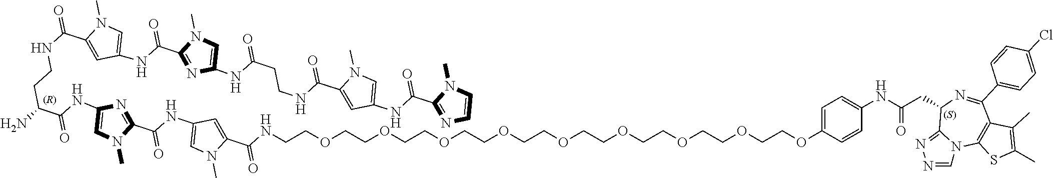 |
| A-36 | 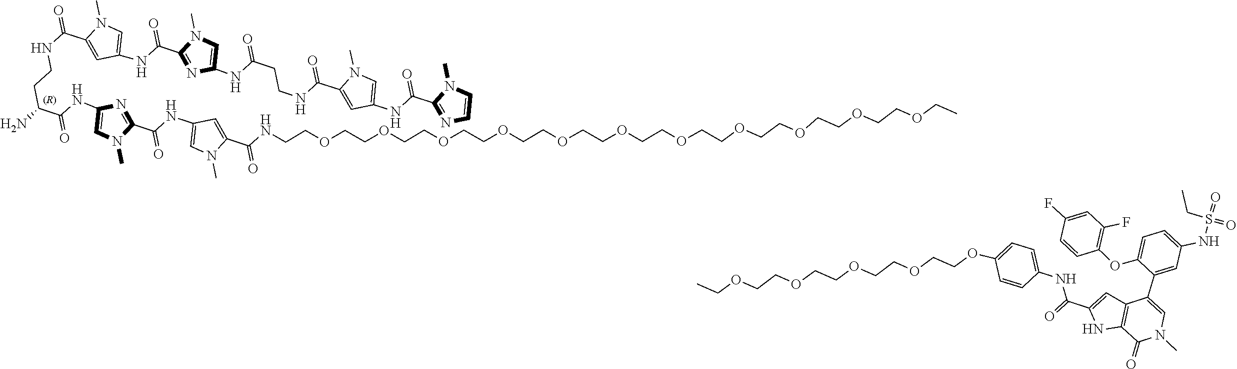 |
| A-37 | 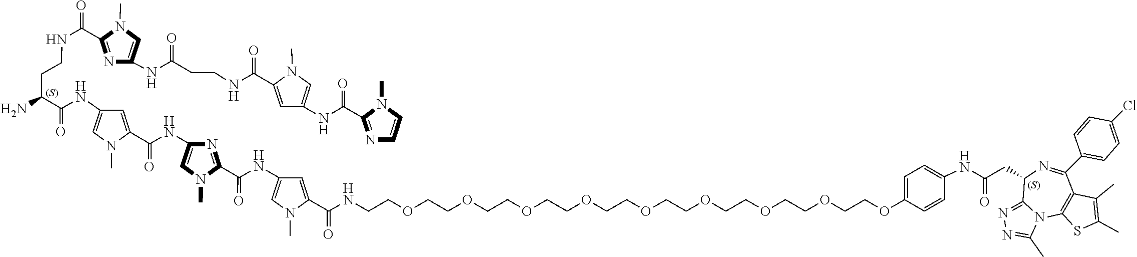 |
| A-38 | 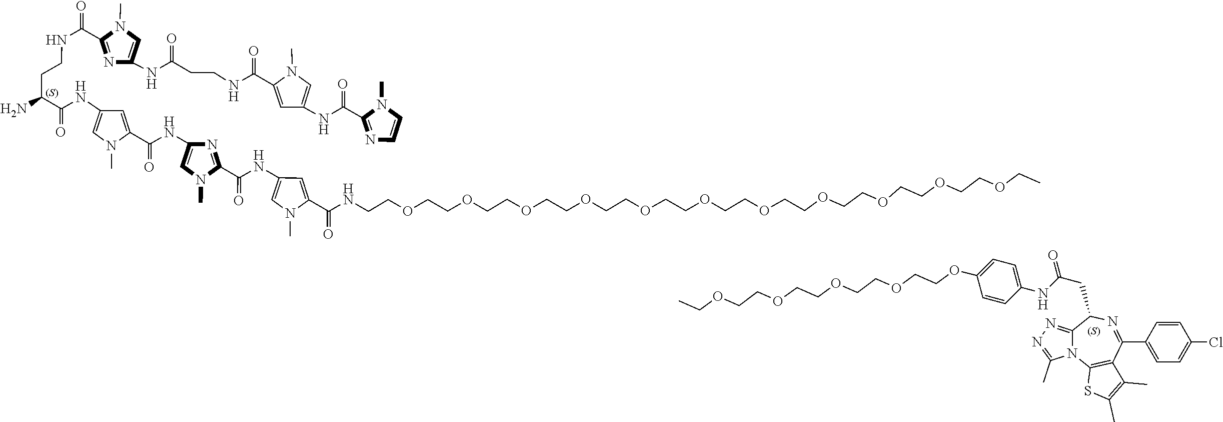 |
| A-39 | 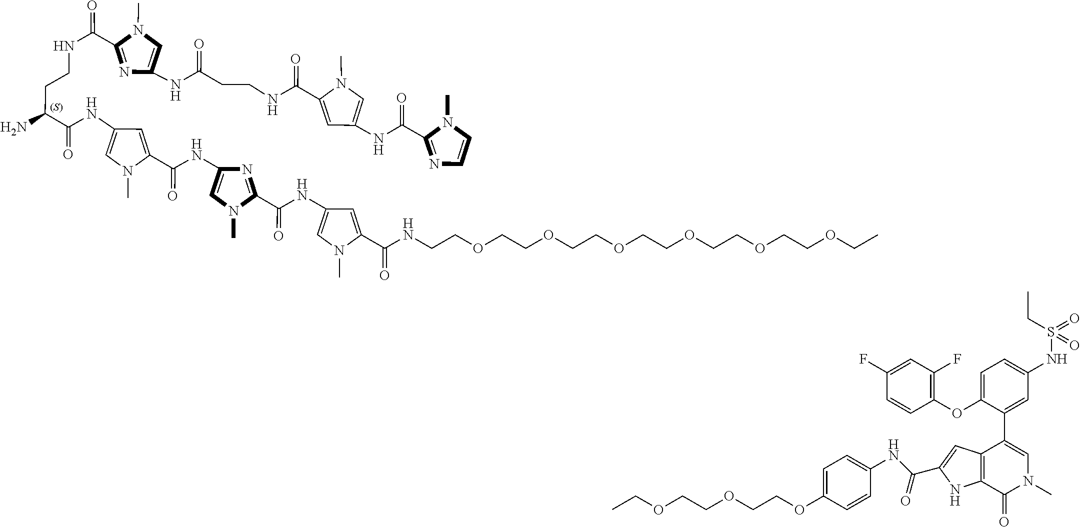 |
| A-40 | 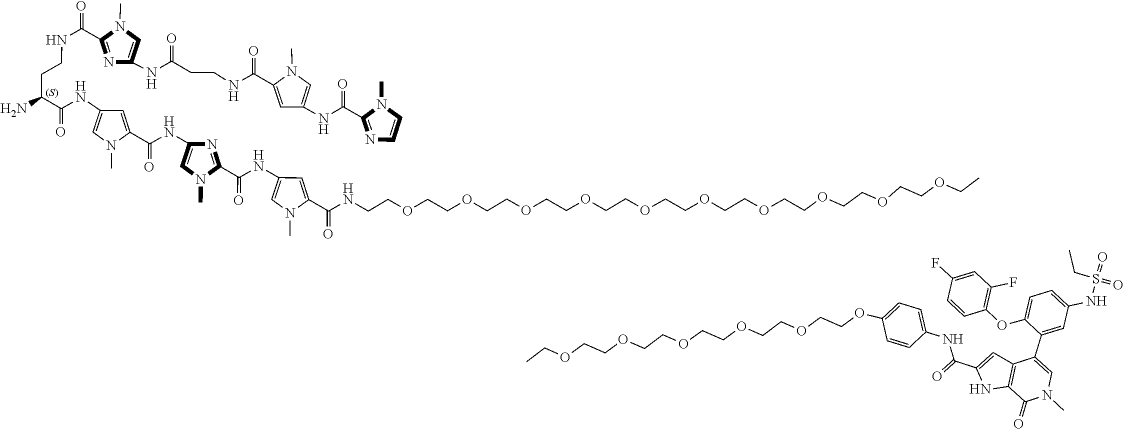 |
| A-41 | 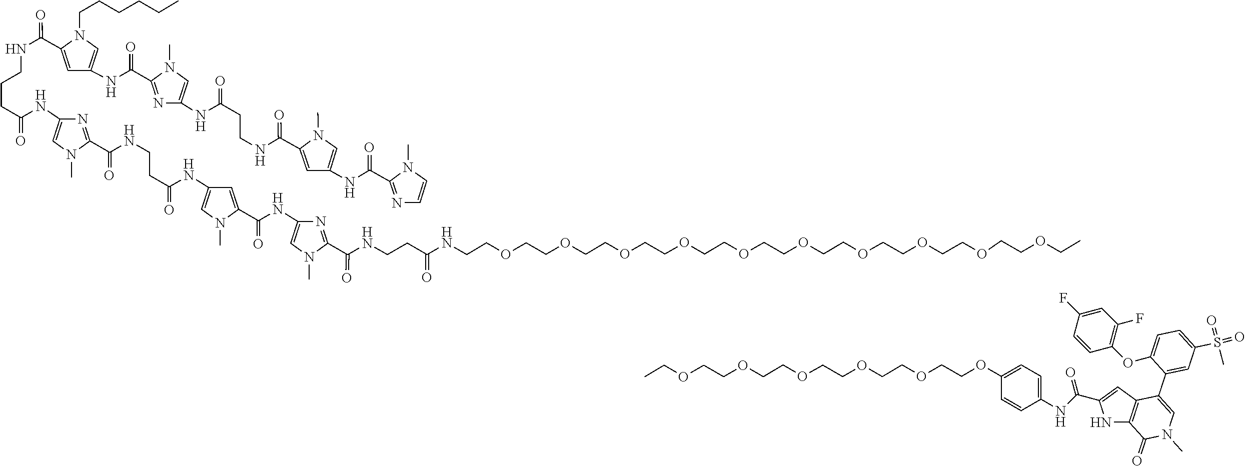 |
| A-42 | 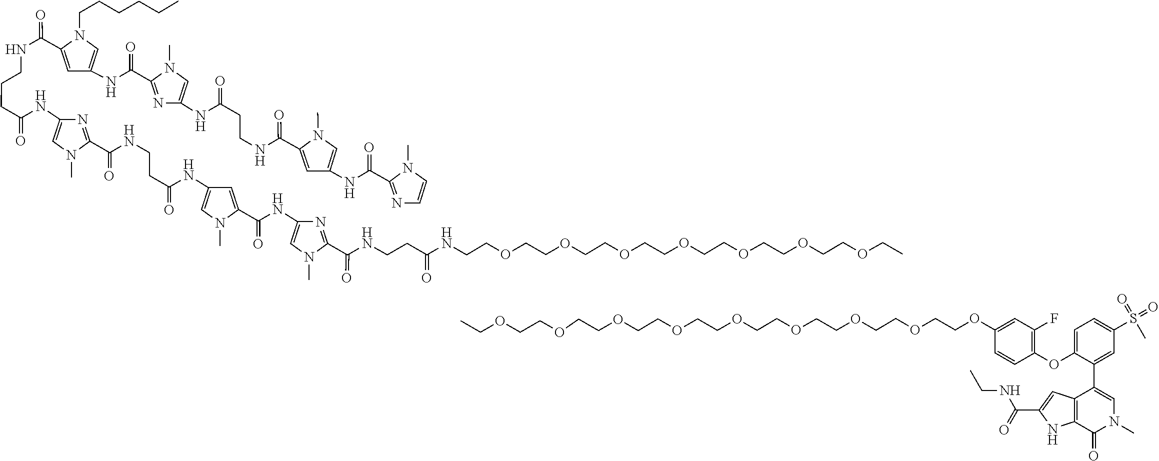 |
| A-43 | 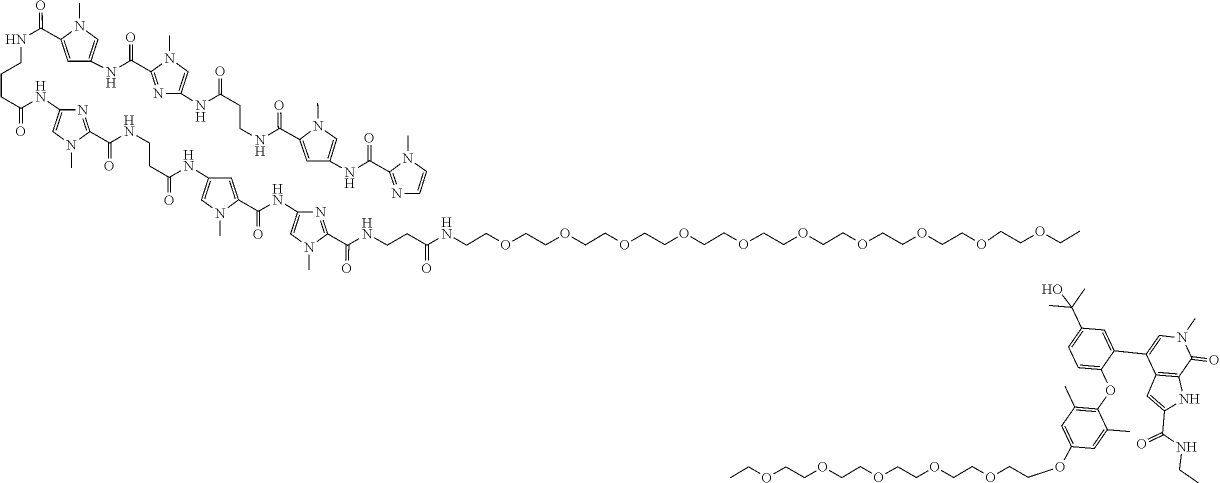 |
| A-44 | 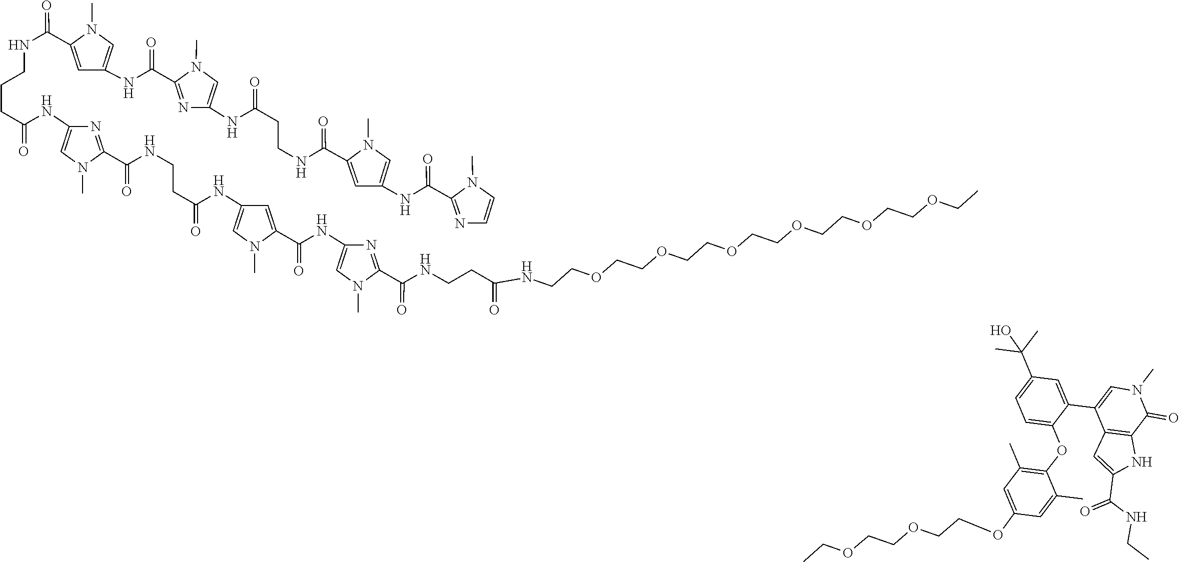 |
| A-45 | 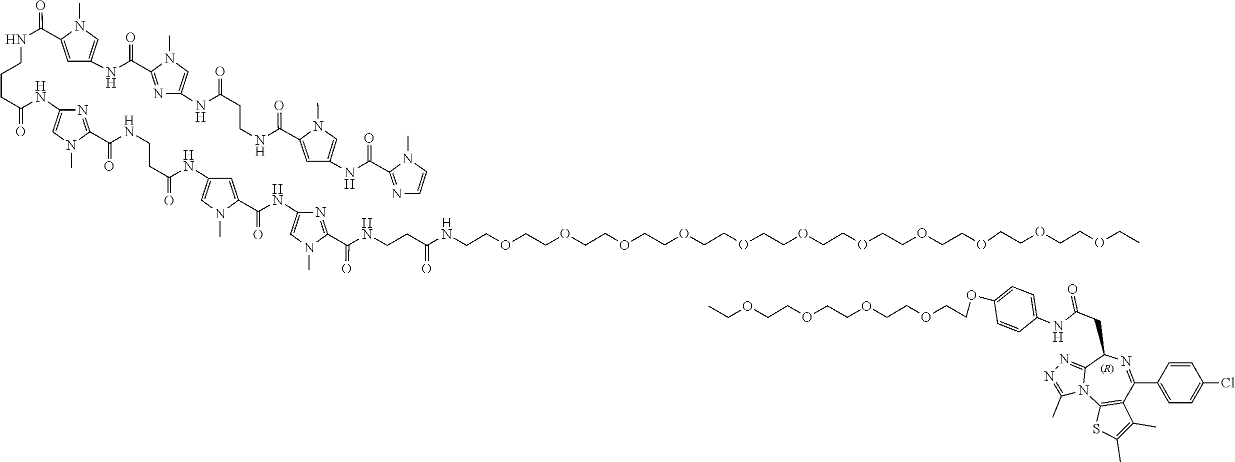 |
| A-46 | 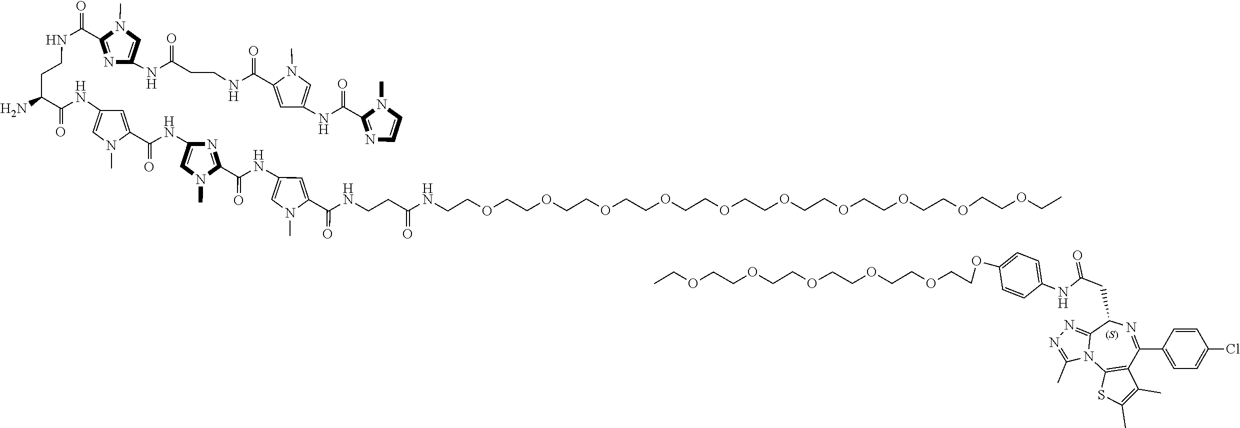 |
| A-47 | 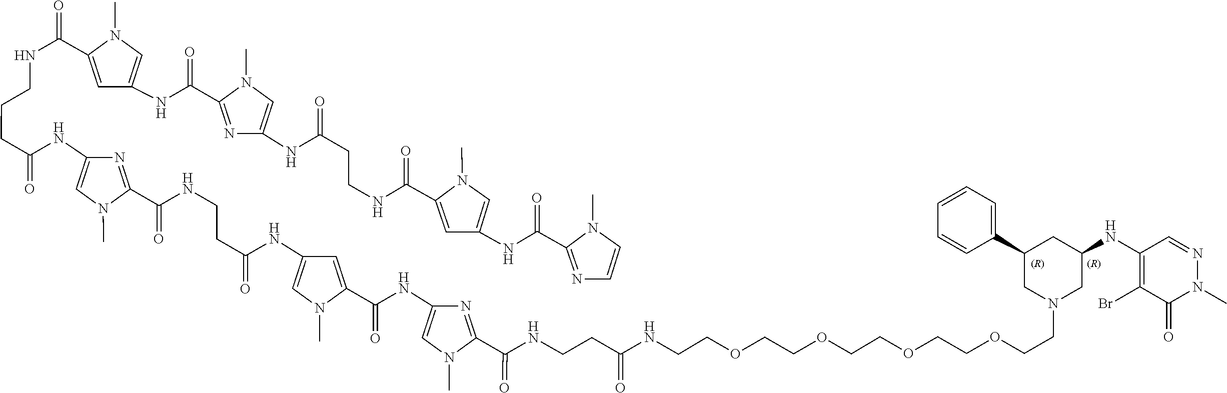 |
| A-48 | 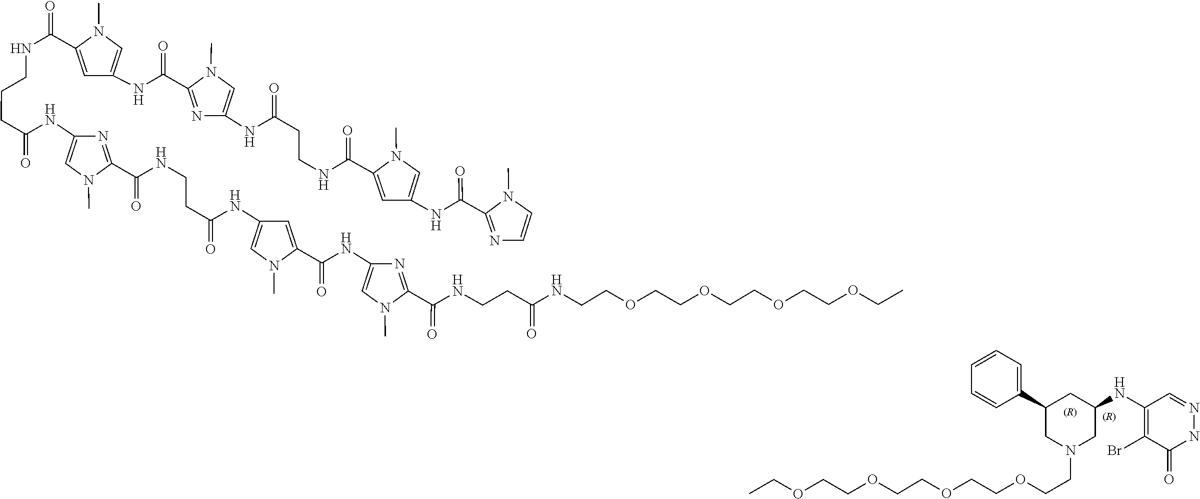 |
| A-49 | 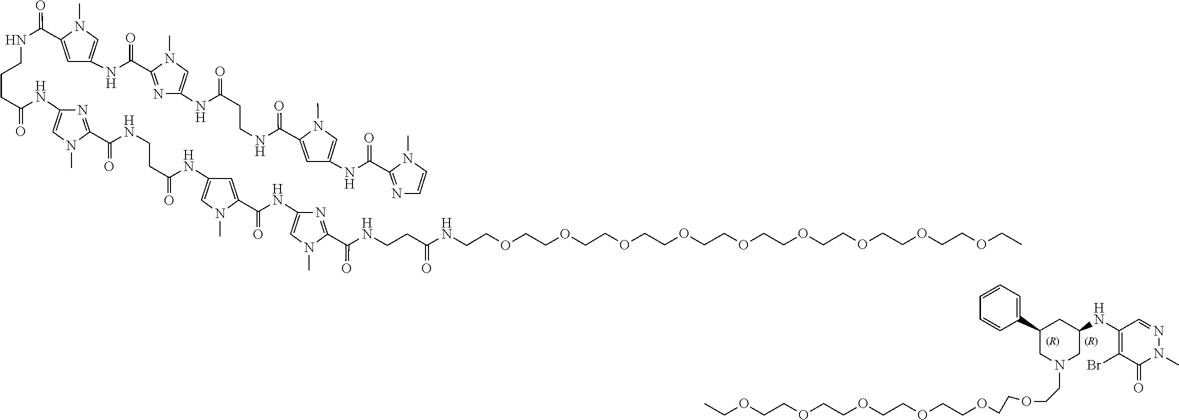 |
| A-50 | 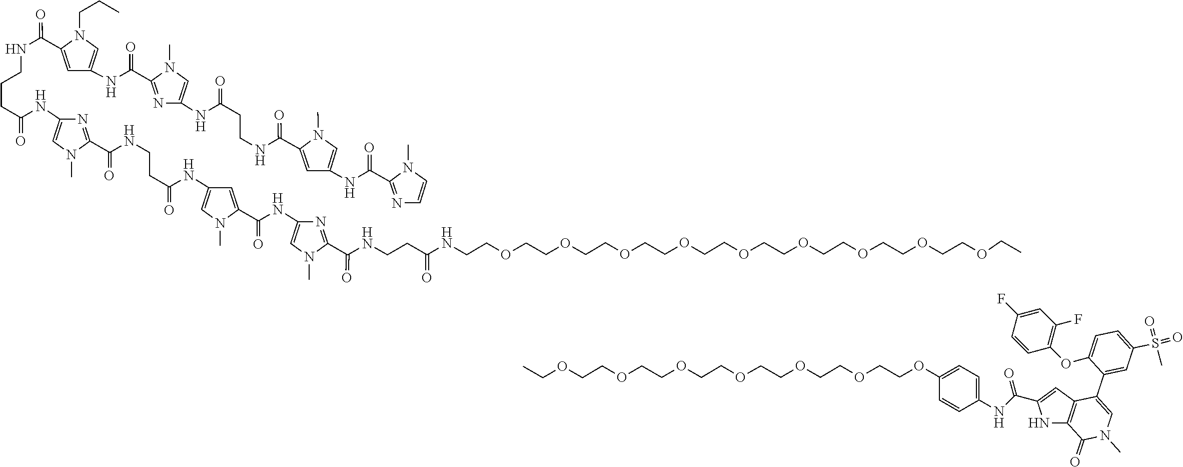 |
| A-51 | 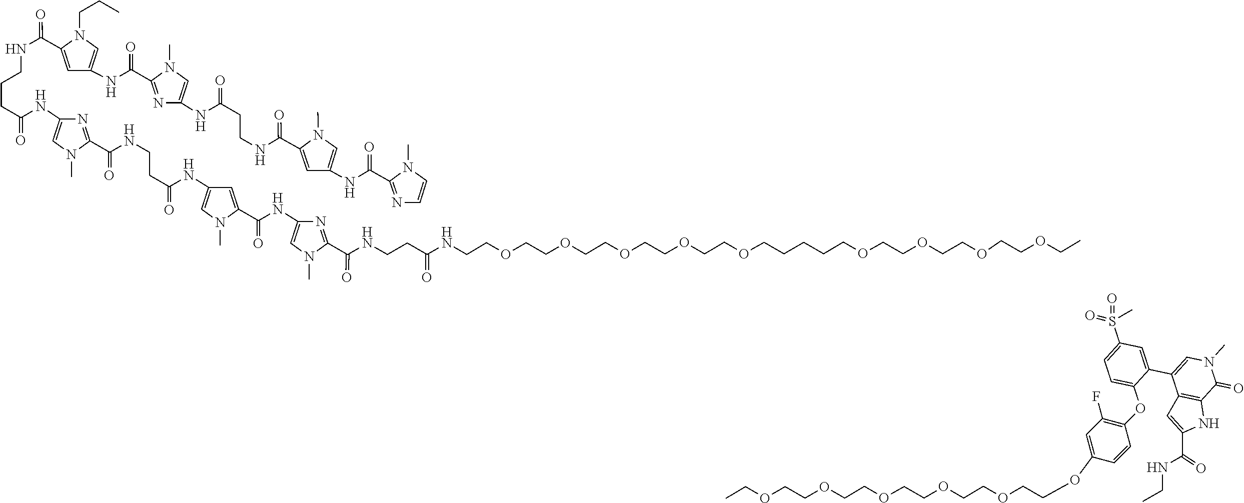 |
| A-52 | 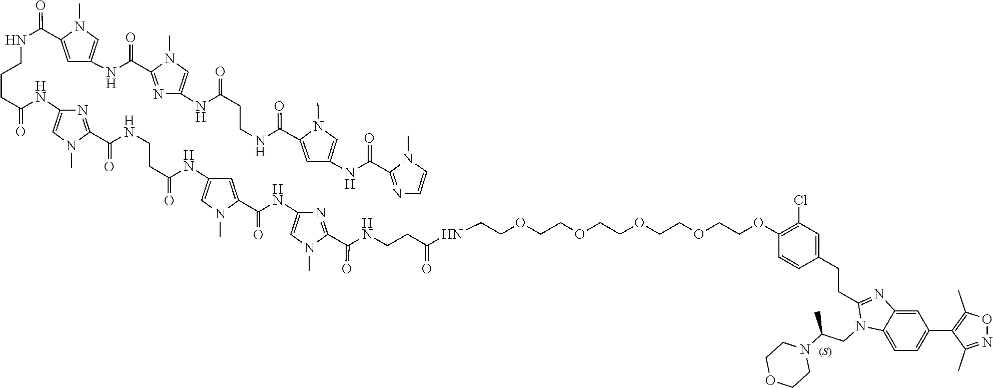 |
| A-53 | 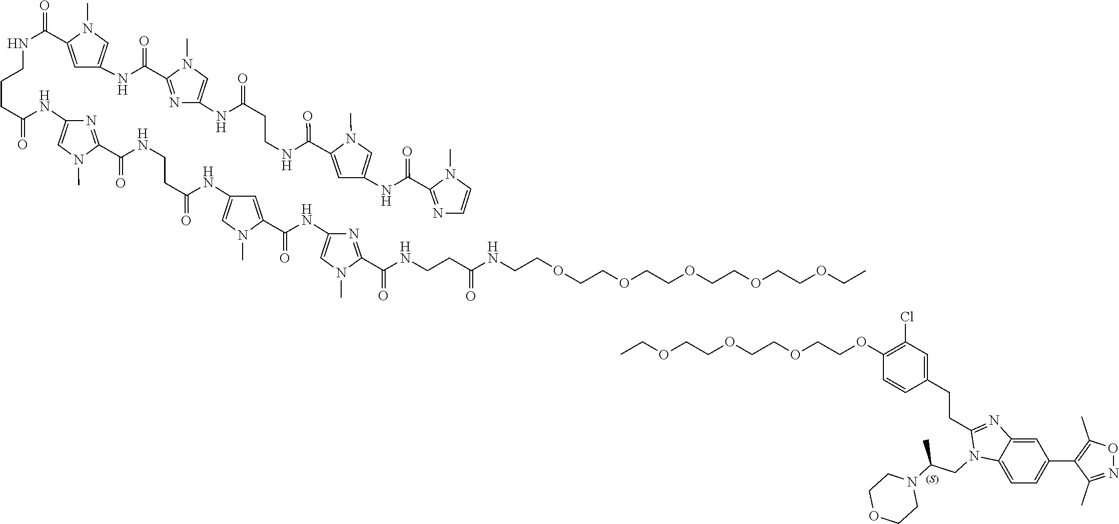 |
| A-54 | 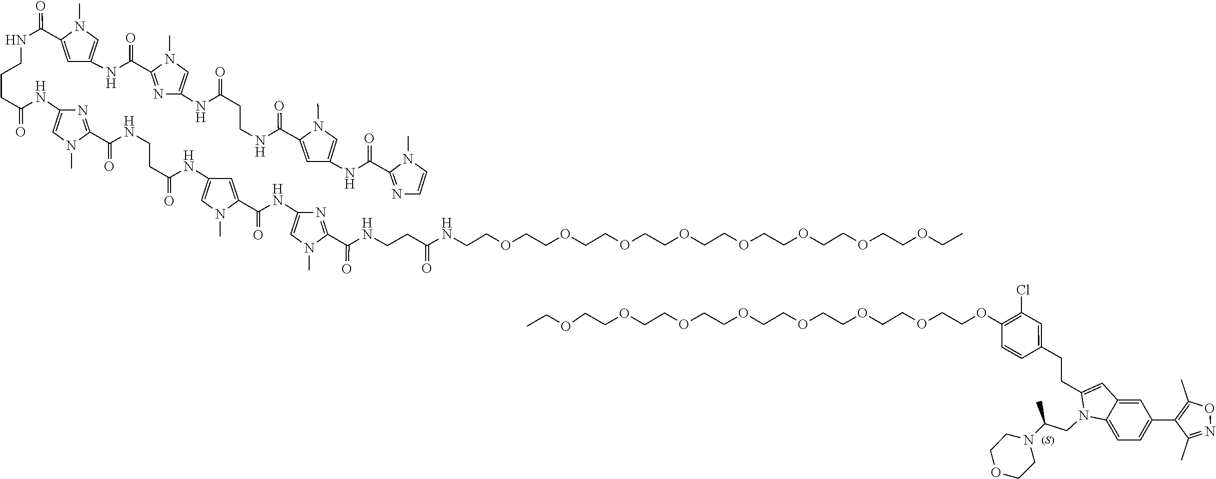 |
| A-55 | 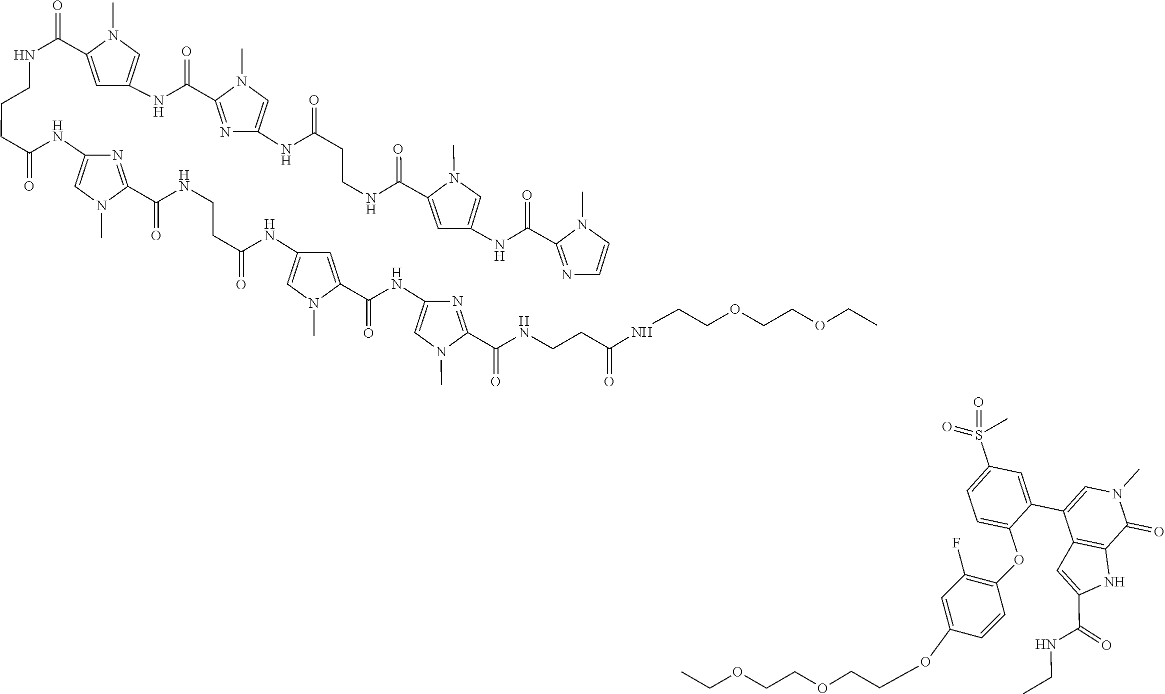 |
| A-56 | 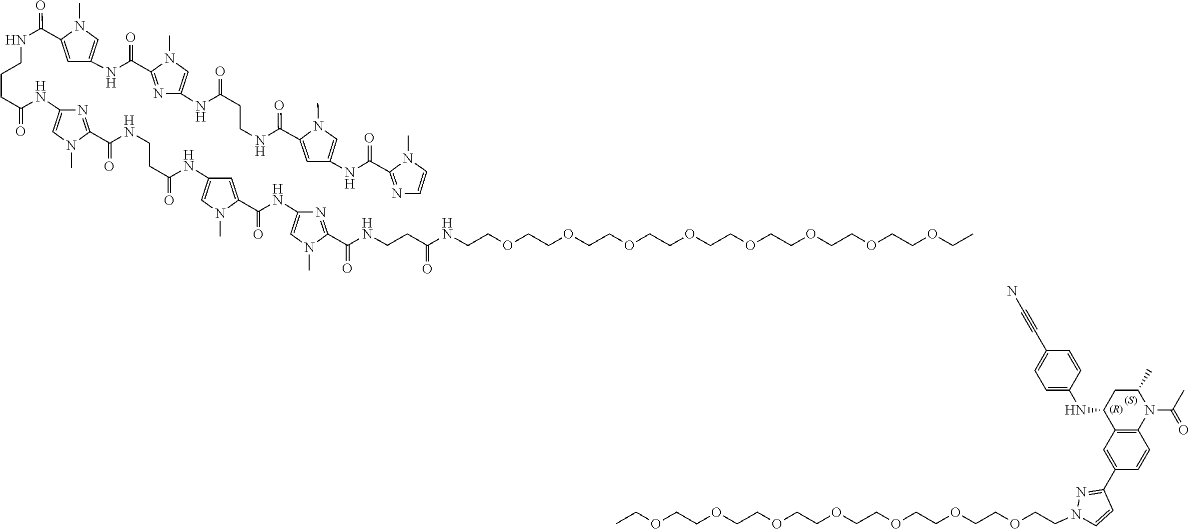 |
| A-57 | 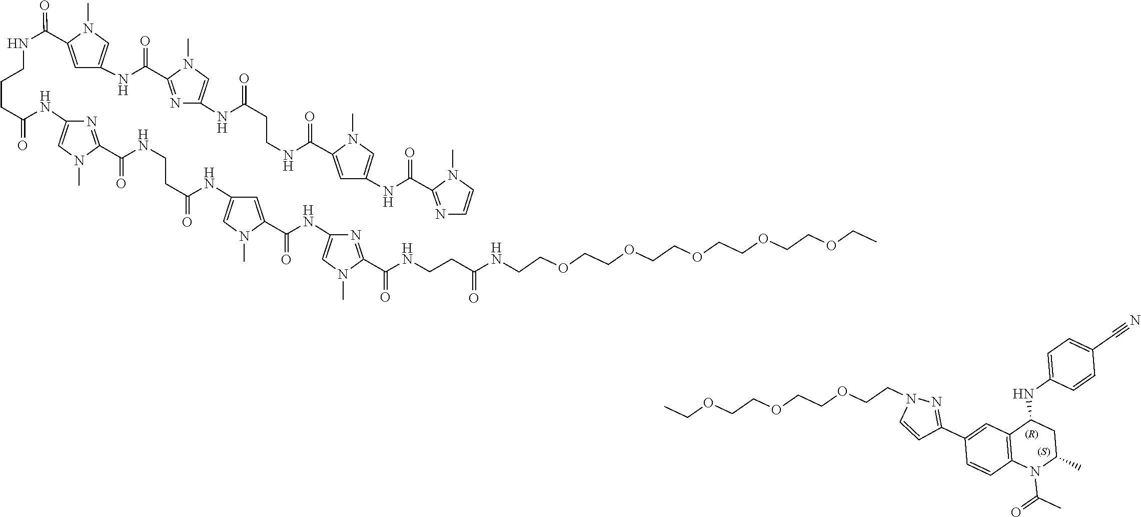 |
| A-58 | 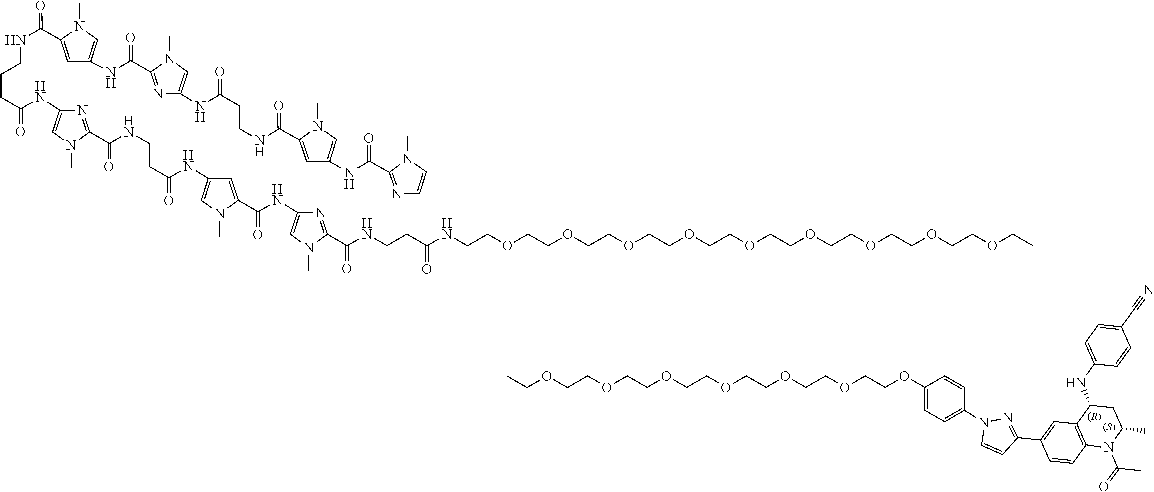 |
| A-59 | 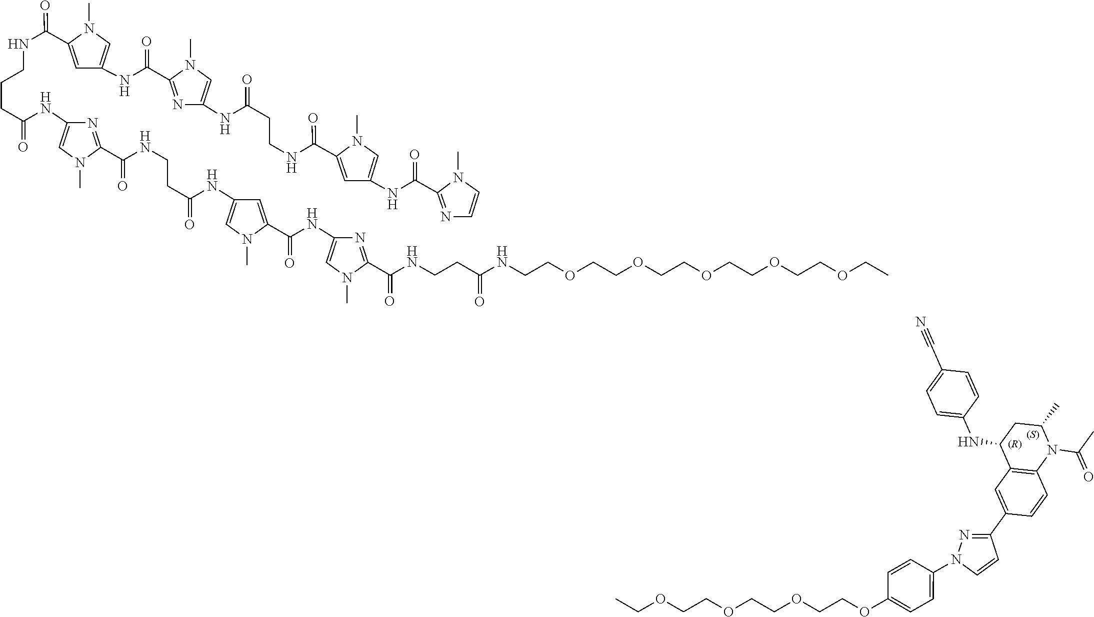 |
| A-60 | 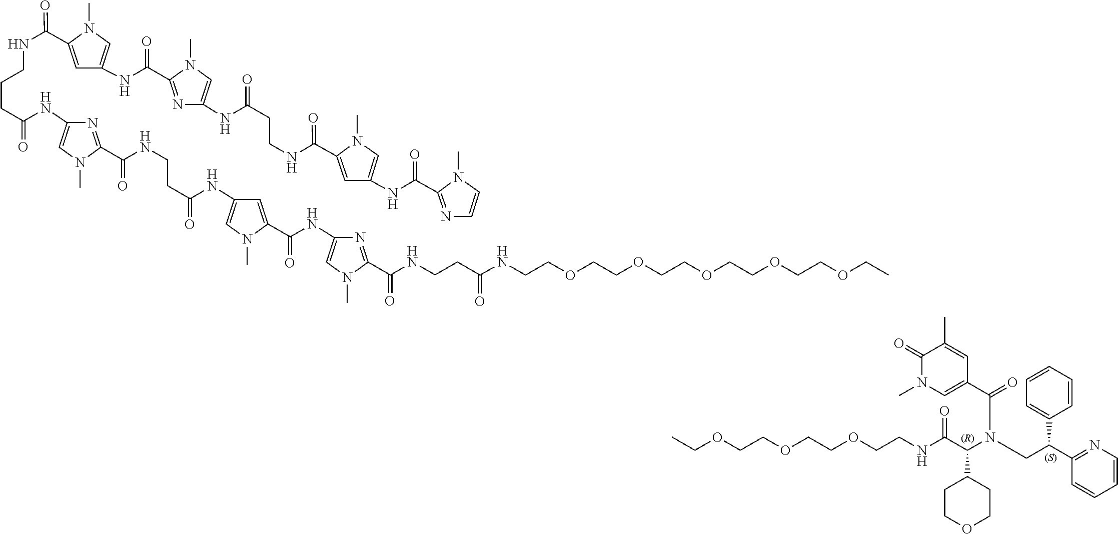 |
| A-61 | 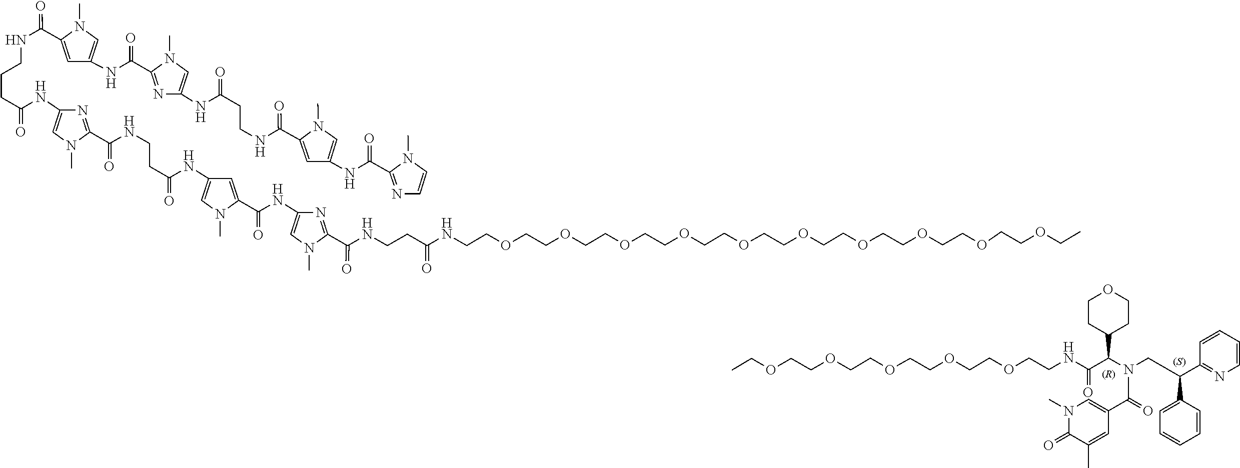 |
| A-62 | 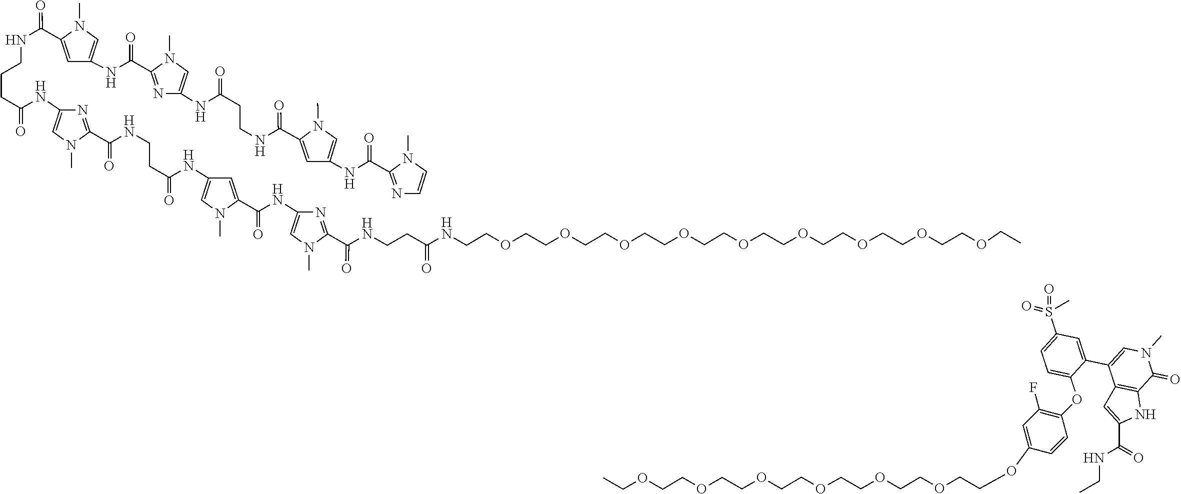 |
| A-63 | 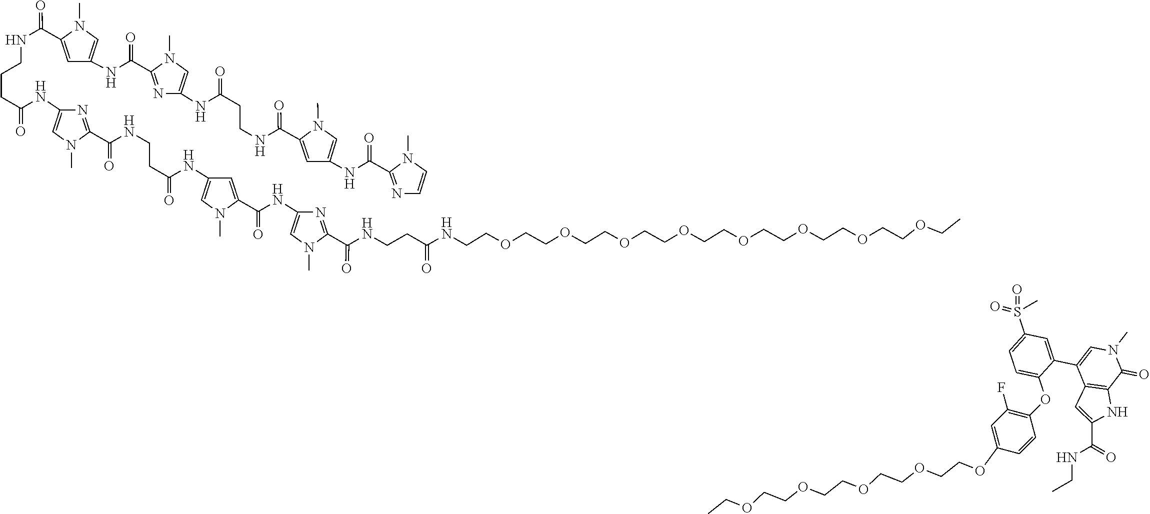 |
| A-64 | 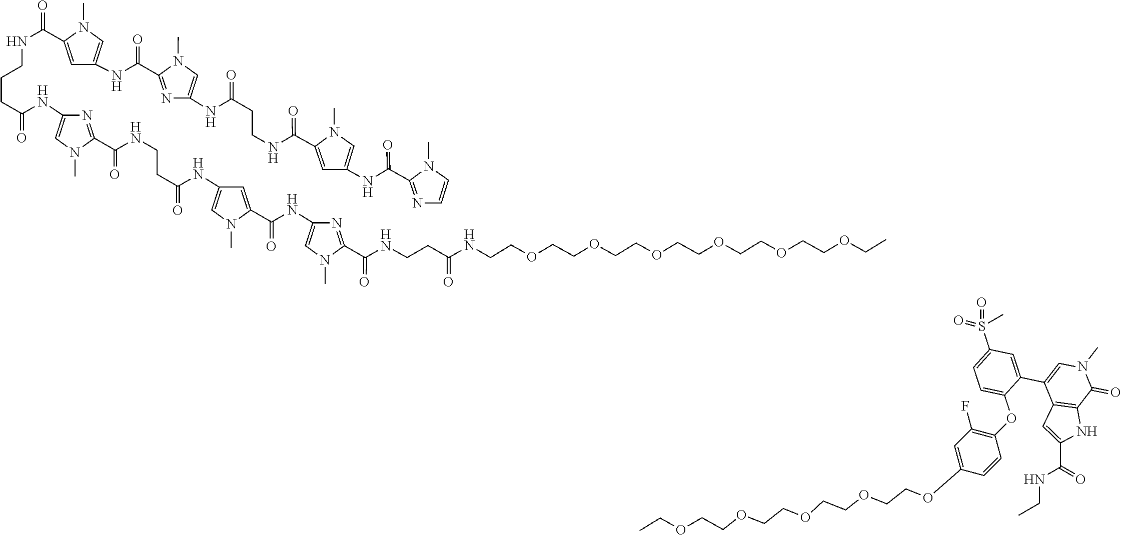 |
| A-65 | 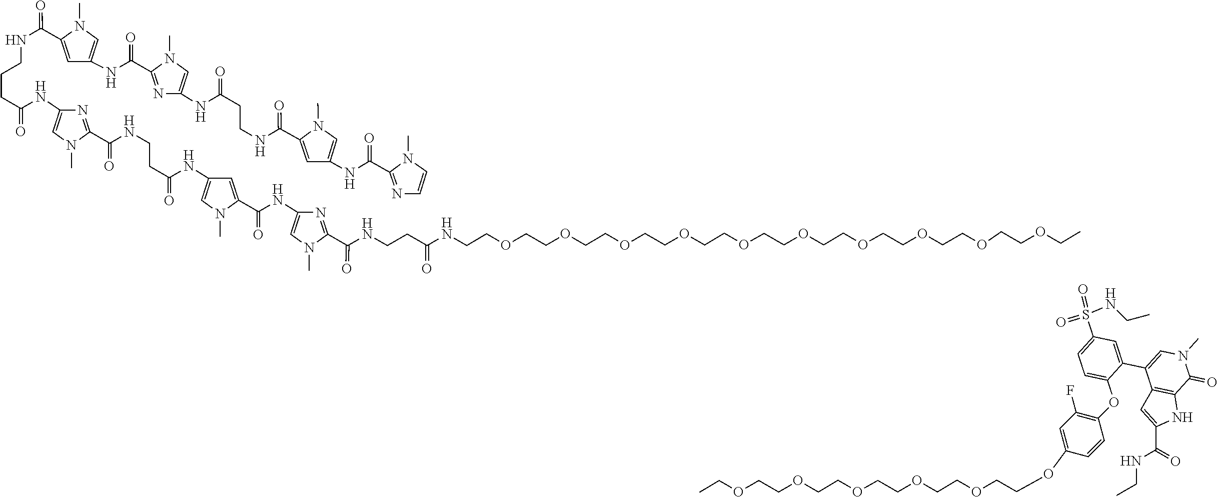 |
| A-66 | 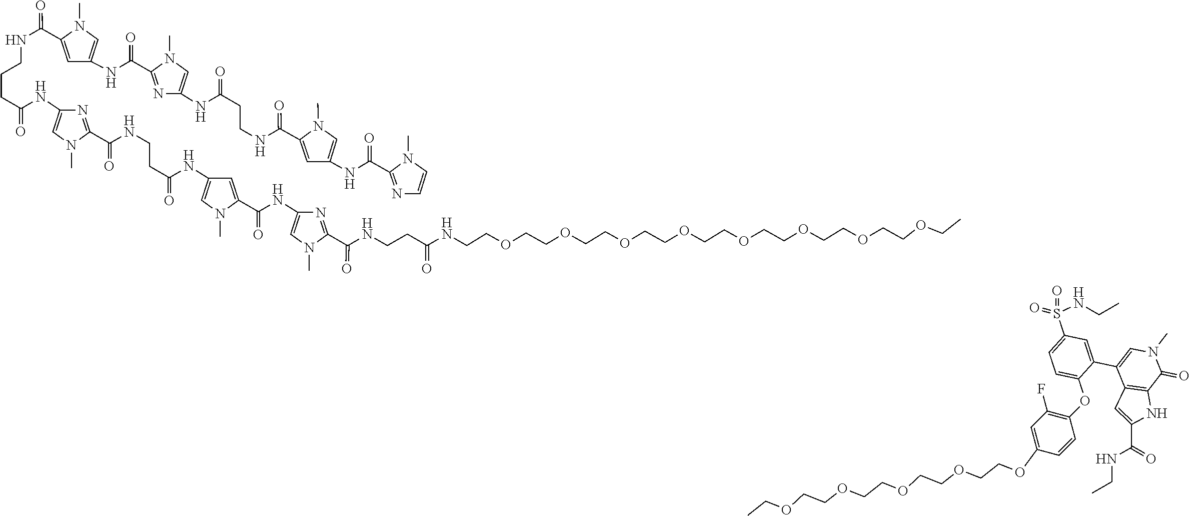 |
| A-67 | 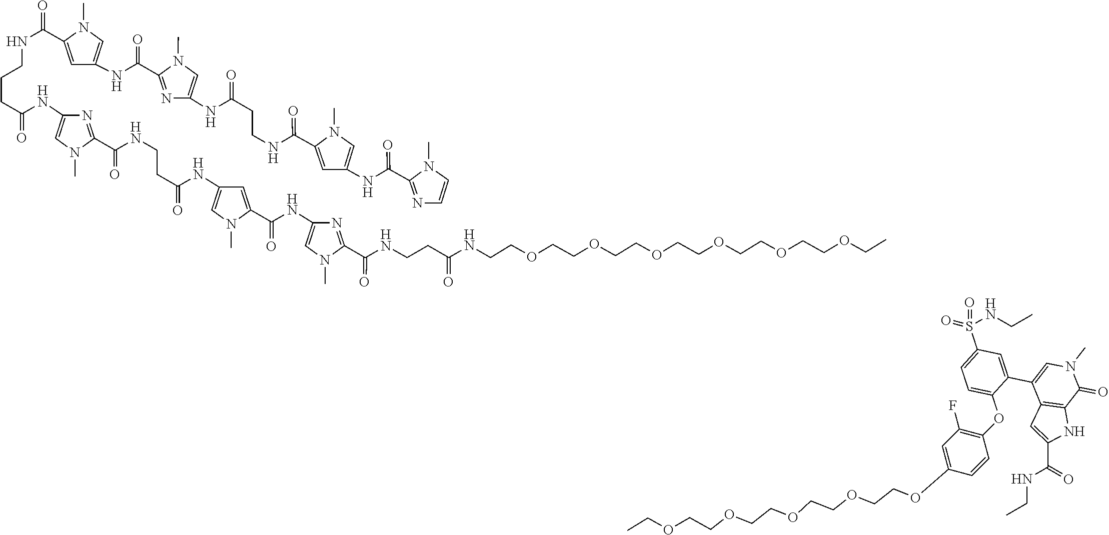 |
| A-68 | 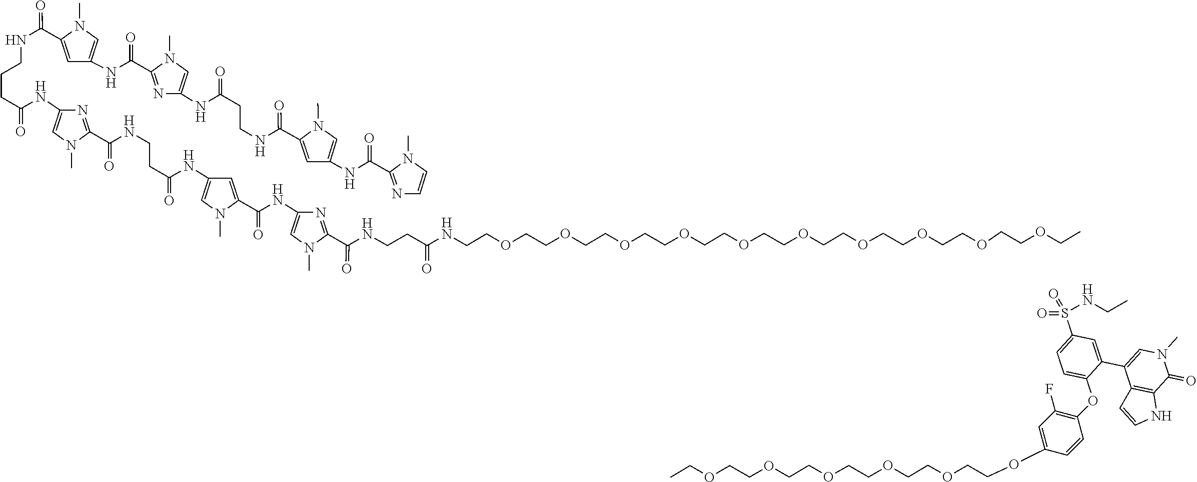 |
| A-69 | 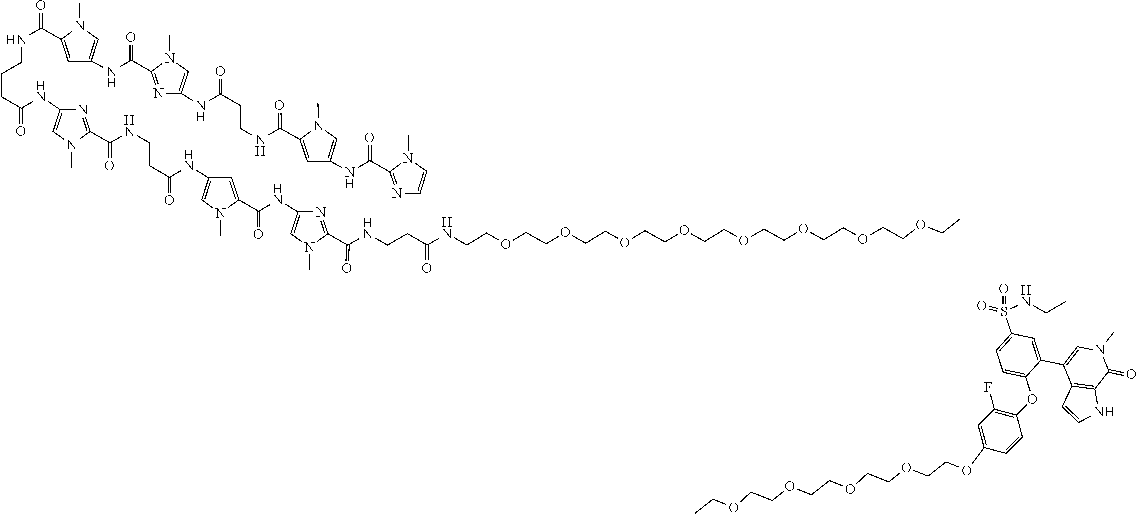 |
| A-70 | 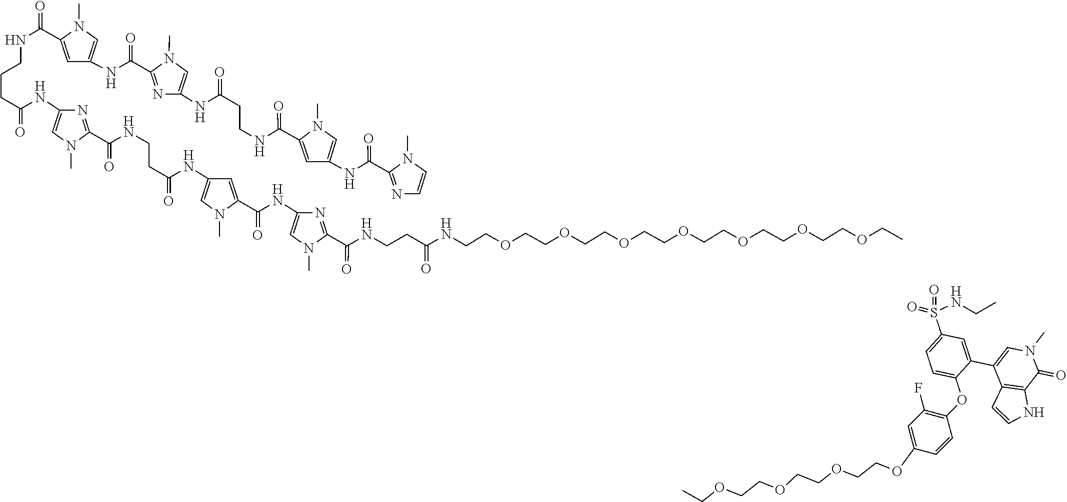 |
| A-71 | 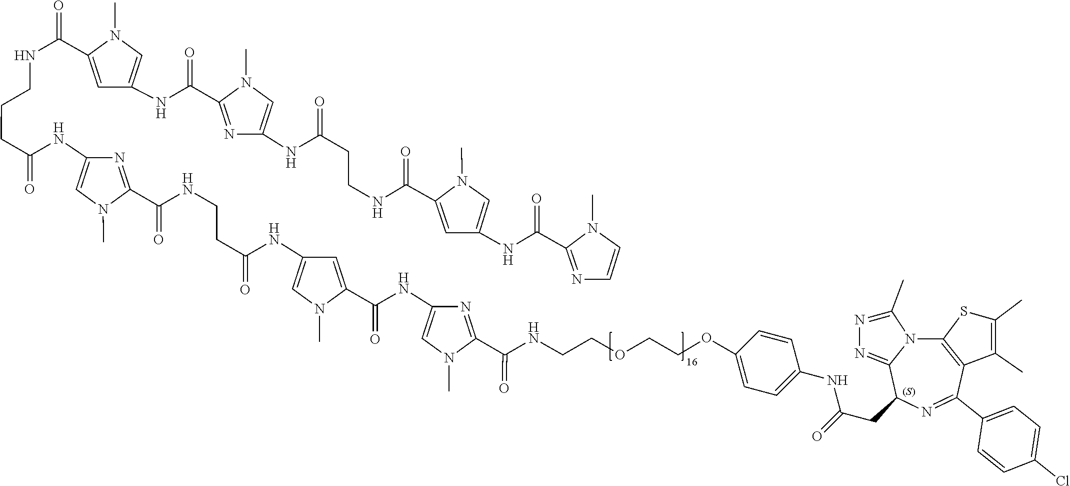 |
| A-72 | 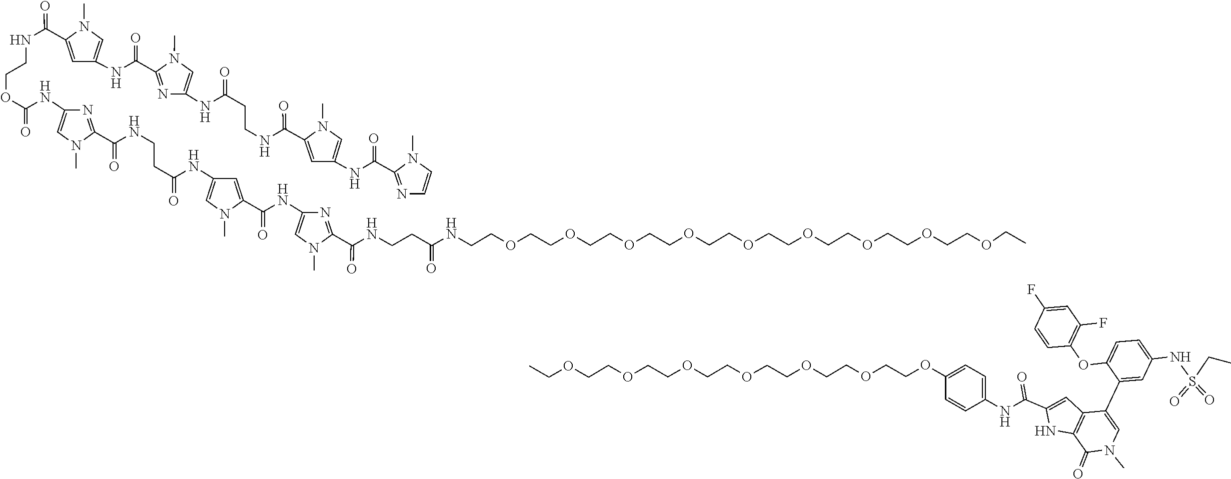 |
| A-73 | 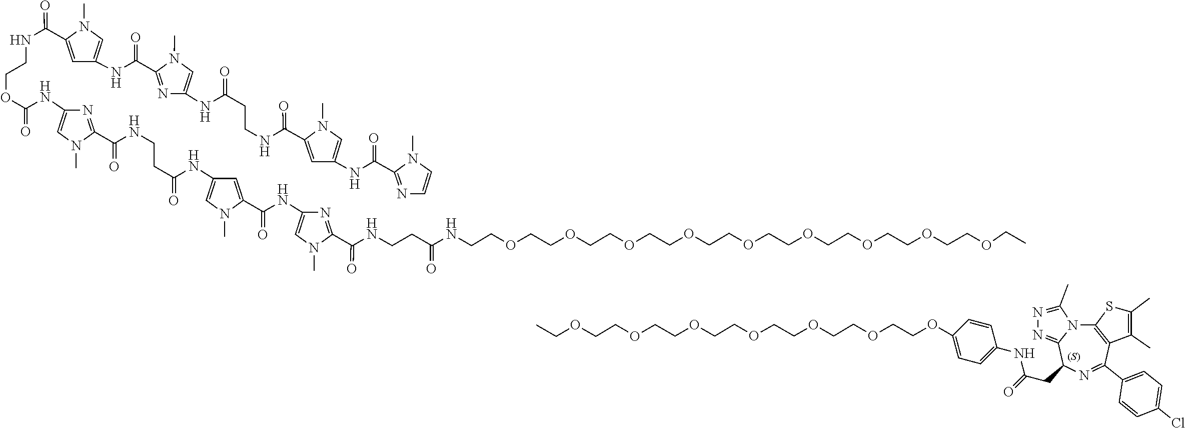 |
| A-74 | 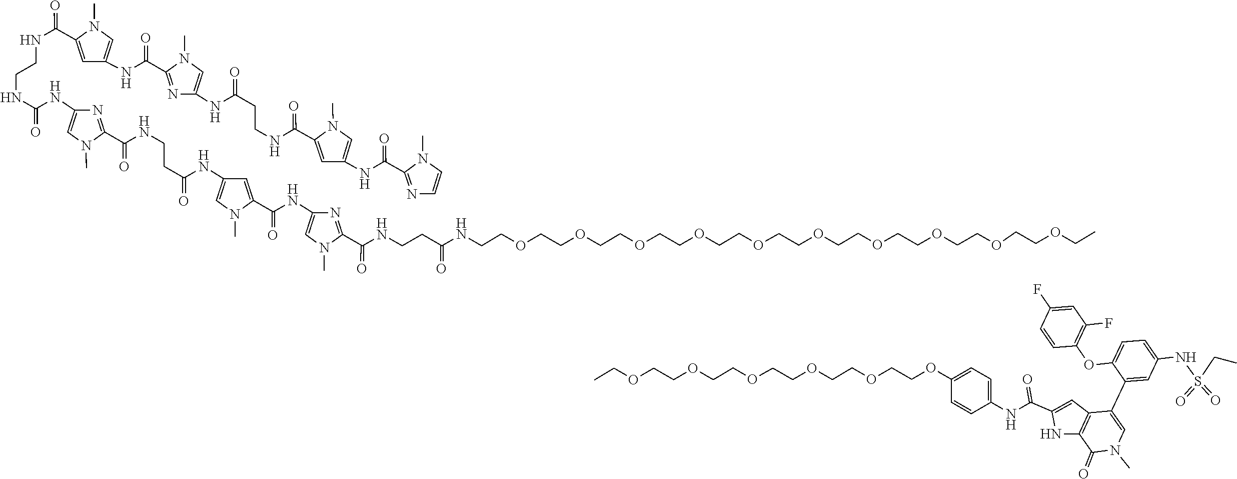 |
| A-75 | 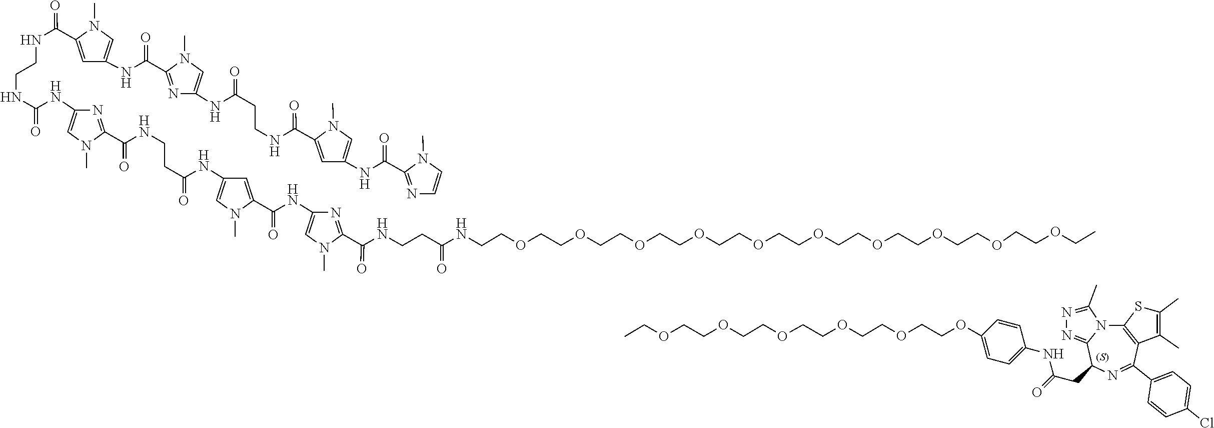 |

As used herein, two embodiments are “mutually exclusive” when one is defined to be something which is different than the other. For example, an embodiment wherein two groups combine to form a cycloalkyl are mutually exclusive with an embodiment in which one group is ethyl and the other group is hydrogen. Similarly, an embodiment wherein one group is CH₂ is mutually exclusive with an embodiment wherein the same group is NH.

Methods of Use

In another aspect, provided herein is a method of treating an individual having an expanded nucleotide repeat disorder, such nucleotide repeat comprising CAG, the method comprising administering a transcriptional modulator molecule having a first terminus, a second terminus, and an oligomeric backbone, wherein
(a) the first terminus comprises a DNA-binding moiety capable of binding a nucleotide repeat comprising CAG;
(b) the second terminus comprises a protein-binding moiety capable of binding to a regulatory molecule that modulates expression of a gene having the expanded nucleotide repeat; and
(c) the oligomeric backbone links the first terminus and the second terminus.

In another aspect, provided herein is a method of decreasing expression of a gene having an expanded nucleotide repeat, such as CAG, in a cell, the method comprising contacting the cell with a transcriptional modulator molecule having a first terminus, a second terminus, and an oligomeric backbone, wherein
(a) the first terminus comprises a DNA-binding moiety capable of binding a nucleotide repeat comprising CAG;
(b) the second terminus comprises a protein-binding moiety capable of binding to a regulatory molecule that modulates expression of a gene having the expanded nucleotide repeat; and
(c) the oligomeric backbone links the first terminus and the second terminus.

In some embodiments, the expanded nucleotide repeat disorder is an expanded CAG repeat disorder.

In some embodiments, the expanded nucleotide repeat disorder is Huntington's disease (HD). In some embodiments, the expanded nucleotide repeat disorder is a Huntington's disease-like syndrome. In some embodiments, the expanded nucleotide repeat disorder is Juvenile Huntington's disease.

In some embodiments, the expanded nucleotide repeat has at least about 36 repeats, at least about 40 repeats, at least about 50 repeats, at least about 60 repeats, at least about 70 repeats, at least about 80 repeats, at least about 90 repeats, at least about 100 repeats, at least about 110 repeats, at least about 120 repeats, or more.

In some embodiments, the expanded nucleotide repeat comprises CAG.

In some embodiments, the method results in reduction of expression of the gene having the expanded nucleotide repeat. In some embodiments, the reduction of expression is at least about 10%, at least about 20%, at least about 30%, at least about 40%, at least about 50%, at least about 60%, at least about 70%, at least about 80%, at least about 90%, at least about 95%, or more, compared to an untreated individual.

In some embodiments, the gene is huntingtin (HTT).

In another aspect, provide herein is a method of treating Huntington's disease (HD) in a patient in need thereof, the method comprising administering to the patient a transcriptional modulator molecule having a first terminus, a second terminus, and an oligomeric backbone, wherein
(a) the first terminus comprises a DNA-binding moiety capable of binding a nucleotide repeat comprising CAG;
(b) the second terminus comprises a protein-binding moiety capable of binding to a regulatory molecule that modulates expression of a gene having the expanded nucleotide repeat; and
(c) the oligomeric backbone links the first terminus and the second terminus; and
   wherein the DNA-binding moiety comprises —NH-Q-C(O)—, wherein Q is an optionally substituted C₆₋₁₀ arylene, optionally substituted 4 to 10-membered heterocyclene, optionally substituted 5 to 10-membered heteroarylene group, or an optionally substituted alkylene group.

In some embodiments, the DNA-binding moiety comprise a polyamide.

In some embodiments, the DNA-binding moiety comprises a polyamide of one or more of the following subunits selected from

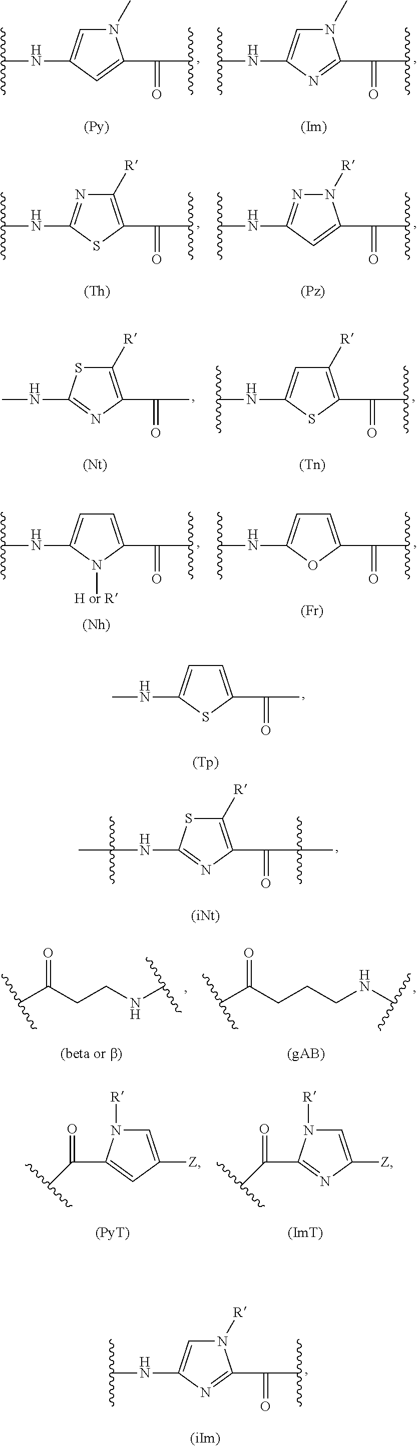

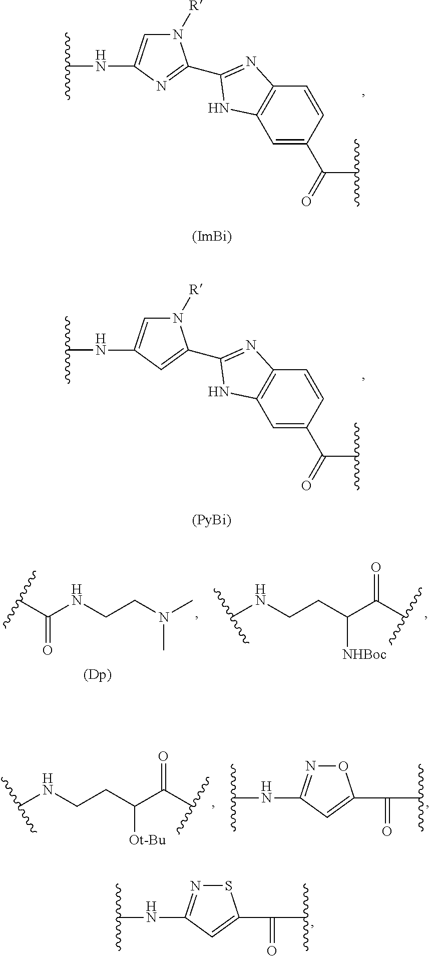

—NH-benzopyrazinylene-C(O)—, —NH-phenylene-C(O)—, —NH-pyridinylene-(C)O—, —NH-piperidinylene-C(O)—, —NH-pyrimidinylene-C(O)—, —NH-anthracenylene-C(O)—, —NH-quinolinylene-C(O)—, and

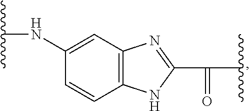

wherein each R′ is independently hydrogen, optionally substituted C₁-C₂₀ alkyl, C₁-C₂₀ heteroalkyl, C₁-C₂₀ haloalkyl, or C₁-C₂₀ alkylamino; and Z is H, NH₂, C₁-C₆ alkyl, C₁-C₆ haloalkyl, or C₁-C₆ alkyl-NH₂.

In some embodiments, the DNA-binding moiety comprises a structure of any one of Formulas (A-1)-(A-13).

In some embodiments, the method reduces one or more symptoms of Huntington's disease.

In some embodiments, the one or more symptoms are selected from chorea, cognitive decline, abnormal libido, abnormal eye movement, abnormal sense of smell, aggression, agitation, anxiety, apathy, bradykinesia, bradyphrenia, clumsiness, delusions, depression, difficulty walking, disinhibition, dystonia, gait imbalance, muscle weakness, hallucinations, hostility, hypokinesia, irritability, memory impairment, myoclonus, obsessive-compulsive behavior, poor fine motor coordination, seizure, speech articulation difficulties, staring gaze, weight loss, abnormal cholesterol metabolism, abnormal cerebral white matter, alcoholism, Babinski sign, caudate atrophy, cerebral atrophy, choking, clonus, degeneration of the striatum, excessive daytime sleepiness, impaired visuospatial constructive cognition, inability to walk, insomnia, mutism, oral-pharyngeal dysphagia, rigidity, suicidal ideation, cerebellar atrophy, dementia, gate ataxia, gliosis, hyperreflexia, neuronal loss, or personality changes.

Pharmaceutical Compositions and Administration

Provided herein, in some embodiments, are compositions comprising a therapeutically effective amount of a transcription modulator molecule described herein (also referred to herein as “a pharmaceutical agent”).

Pharmaceutical compositions may be formulated using one or more physiologically acceptable carriers including excipients and auxiliaries which facilitate processing of the pharmaceutical agent into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa., Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

The compositions and methods of the present disclosure may be utilized to treat an individual in need thereof. In certain embodiments, the individual is a mammal such as a human, or a non-human mammal. When administered to an animal, such as a human, the composition or the pharmaceutical agent, is preferably administered as a pharmaceutical composition comprising, for example, a pharmaceutical agent and a pharmaceutically acceptable carrier or excipient. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration, e.g., routes, such as injection or implantation, that circumvent transport or diffusion through an epithelial barrier, the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule, granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable excipient can contain physiologically acceptable agents that act, for example, to stabilize, increase solubility or to increase the absorption of a compound such as a pharmaceutical agent. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable excipient, including a physiologically acceptable agent, depends, for example, on the route of administration of the composition. The preparation or pharmaceutical composition can be a self-emulsifying drug delivery system or a self microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

A pharmaceutical composition (preparation) can be administered to a subject by any of a number of routes of administration including, for example, orally, for example, drenches as in aqueous or non-aqueous solutions or suspensions, tablets, capsules, including sprinkle capsules and gelatin capsules, boluses, powders, granules, pastes for application to the tongue: absorption through the oral mucosa, e.g., sublingually; anally, rectally or vaginally, for example, as a pessary, cream or foam; parenterally, including intramuscularly, intravenously, subcutaneously or intrathecally as, for example, a sterile solution or suspension; nasally; intraperitoneally; subcutaneously; transdermally, for example, as a patch applied to the skin: and topically, for example, as a cream, ointment or spray applied to the skin, or as an eye drop. The compound may also be formulated for inhalation. In certain embodiments, a compound may be simply dissolved or suspended in sterile water.

A pharmaceutical composition may be a sterile aqueous or non-aqueous solution, suspension or emulsion, e.g., a microemulsion. The excipients described herein are examples and are in no way limiting. An effective amount or therapeutically effective amount refers to an amount of the one or more pharmaceutical agents administered to a subject, either as a single dose or as part of a series of doses, which is effective to produce a desired therapeutic effect.

Subjects may generally be monitored for therapeutic effectiveness using assays and methods suitable for the condition being treated, which assays will be familiar to those having ordinary skill in the art and are described herein. Pharmacokinetics of a pharmaceutical agent, or one or more metabolites thereof, that is administered to a subject may be monitored by determining the level of the pharmaceutical agent or metabolite in a biological fluid, for example, in the blood, blood fraction, e.g., serum, and/or in the urine, and/or other biological sample or biological tissue from the subject. Any method practiced in the art and described herein to detect the agent may be used to measure the level of the pharmaceutical agent or metabolite during a treatment course.

The dose of a pharmaceutical agent described herein for treating a disease or disorder may depend upon the subject's condition, that is, stage of the disease, severity of symptoms caused by the disease, general health status, as well as age, gender, and weight, and other factors apparent to a person skilled in the medical art. Pharmaceutical compositions may be administered in a manner appropriate to the disease to be treated as determined by persons skilled in the medical arts. In addition to the factors described herein and above related to use of pharmaceutical agent for treating a disease or disorder, suitable duration and frequency of administration of the pharmaceutical agent may also be determined or adjusted by such factors as the condition of the patient, the type and severity of the patient's disease, the particular form of the active ingredient, and the method of administration. Optimal doses of an agent may generally be determined using experimental models and/or clinical trials. The optimal dose may depend upon the body mass, weight, or blood volume of the subject. The use of the minimum dose that is sufficient to provide effective therapy is usually preferred. Design and execution of pre-clinical and clinical studies for a pharmaceutical agent, including when administered for prophylactic benefit, described herein are well within the skill of a person skilled in the relevant art. When two or more pharmaceutical agents are administered to treat a disease or disorder, the optimal dose of each pharmaceutical agent may be different, such as less than when either agent is administered alone as a single agent therapy. In certain particular embodiments, two pharmaceutical agents in combination may act synergistically or additively, and either agent may be used in a lesser amount than if administered alone. An amount of a pharmaceutical agent that may be administered per day may be, for example, between about 0.01 mg/kg and 100 mg/kg, e.g., between about 0.1 to 1 mg/kg, between about 1 to 10 mg/kg, between about 10-50 mg/kg, between about 50-100 mg/kg body weight. In other embodiments, the amount of a pharmaceutical agent that may be administered per day is between about 0.01 mg/kg and 1000 mg/kg, between about 100-500 mg/kg, or between about 500-1000 mg/kg body weight. The optimal dose, per day or per course of treatment, may be different for the disease or disorder to be treated and may also vary with the administrative route and therapeutic regimen.

Pharmaceutical compositions comprising a pharmaceutical agent can be formulated in a manner appropriate for the delivery method by using techniques routinely practiced in the art. The composition may be in the form of a solid, e.g., tablet, capsule, semi-solid, e.g., gel, liquid, or gas, e.g., aerosol. In other embodiments, the pharmaceutical composition is administered as a bolus infusion.

Pharmaceutical acceptable excipients are well known in the pharmaceutical art and described, for example, in Rowe et al., Handbook of Pharmaceutical Excipients: A Comprehensive Guide to Uses, Properties, and Safety, 5^{th} Ed., 2006, and in Remington: The Science and Practice of Pharmacy (Gennaro, 21^{st} Ed. Mack Pub. Co., Easton, PA (2005)). Exemplary pharmaceutically acceptable excipients include sterile saline and phosphate buffered saline at physiological pH. Preservatives, stabilizers, dyes, buffers, and the like may be provided in the pharmaceutical composition. In addition, antioxidants and suspending agents may also be used. In general, the type of excipient is selected based on the mode of administration, as well as the chemical composition of the active ingredient(s). Alternatively, compositions described herein may be formulated as a lyophilizate. A composition described herein may be lyophilized or otherwise formulated as a lyophilized product using one or more appropriate excipient solutions for solubilizing and/or diluting the pharmaceutical agent(s) of the composition upon administration. In other embodiments, the pharmaceutical agent may be encapsulated within liposomes using technology known and practiced in the art. In certain particular embodiments, a pharmaceutical agent is not formulated within liposomes for application to a stent that is used for treating highly, though not totally, occluded arteries. Pharmaceutical compositions may be formulated for any appropriate manner of administration described herein and in the art.

A pharmaceutical composition, e.g., for oral administration or for injection, infusion, subcutaneous delivery, intramuscular delivery, intraperitoneal delivery or other method, may be in the form of a liquid. A liquid pharmaceutical composition may include, for example, one or more of the following: a sterile diluent such as water, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral composition can be enclosed in ampoules, disposable syringes or multiple dose vials made of glass or plastic. The use of physiological saline is preferred, and an injectable pharmaceutical composition is preferably sterile. In another embodiment, for treatment of an ophthalmological condition or disease, a liquid pharmaceutical composition may be applied to the eye in the form of eye drops. A liquid pharmaceutical composition may be delivered orally.

For oral formulations, at least one of the pharmaceutical agents described herein can be used alone or in combination with appropriate additives to make tablets, powders, granules or capsules, and if desired, with diluents, buffering agents, moistening agents, preservatives, coloring agents, and flavoring agents. The pharmaceutical agents may be formulated with a buffering agent to provide for protection of the compound from low pH of the gastric environment and/or an enteric coating. A pharmaceutical agent included in a pharmaceutical composition may be formulated for oral delivery with a flavoring agent, e.g., in a liquid, solid or semi-solid formulation and/or with an enteric coating.

A pharmaceutical composition comprising any one of the pharmaceutical agents described herein may be formulated for sustained or slow release, also called timed release or controlled release. Such compositions may generally be prepared using well known technology and administered by, for example, oral, rectal, intradermal, or subcutaneous implantation, or by implantation at the desired target site. Sustained-release formulations may contain the compound dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of pharmaceutical agent contained within a sustained release formulation depends upon the site of implantation, the rate and expected duration of release, and the nature of the condition, disease or disorder to be treated or prevented.

In certain embodiments, the pharmaceutical compositions comprising a pharmaceutical agent are formulated for transdermal, intradermal, or topical administration. The compositions can be administered using a syringe, bandage, transdermal patch, insert, or syringe-like applicator, as a powder/talc or other solid, liquid, spray, aerosol, ointment, foam, cream, gel, paste. This preferably is in the form of a controlled release formulation or sustained release formulation administered topically or injected directly into the skin adjacent to or within the area to be treated, e.g., intradermally or subcutaneously. The active compositions can also be delivered via iontophoresis. Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, thimerosal, and combinations thereof.

Pharmaceutical compositions comprising a pharmaceutical agent can be formulated as emulsions for topical application. An emulsion contains one liquid distributed in the body of a second liquid. The emulsion may be an oil-in-water emulsion or a water-in-oil emulsion. Either or both of the oil phase and the aqueous phase may contain one or more surfactants, emulsifiers, emulsion stabilizers, buffers, and other excipients. The oil phase may contain other oily pharmaceutically approved excipients. Suitable surfactants include, but are not limited to, anionic surfactants, non-ionic surfactants, cationic surfactants, and amphoteric surfactants. Compositions for topical application may also include at least one suitable suspending agent, antioxidant, chelating agent, emollient, or humectant.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. Liquid sprays may be delivered from pressurized packs, for example, via a specially shaped closure. Oil-in-water emulsions can also be used in the compositions, patches, bandages and articles. These systems are semisolid emulsions, micro-emulsions, or foam emulsion systems.

### Definitions

As used herein, the terms below have the meanings indicated.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene,” “alkenylene,” “arylene”, “heteroarylene.”

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “polyamide” refers to polymers of linkable units chemically bound by amide (i.e., CONH) linkages; optionally, polyamides include chemical probes conjugated therewith. Polyamides may be synthesized by stepwise condensation of carboxylic acids (COOH) with amines (RR′NH) using methods known in the art. Alternatively, polyamides may be formed using enzymatic reactions in vitro, or by employing fermentation with microorganisms.

The term “linkable unit” refers to methylimidazoles, methylpyrroles, and straight and branched chain aliphatic functionalities (e.g., methylene, ethylene, propylene, butylene, and the like) which optionally contain nitrogen substituents, and chemical derivatives thereof. The aliphatic functionalities of linkable units can be provided, for example, by condensation of B-alanine or dimethylaminopropylamine during synthesis of the polyamide by methods well known in the art.

The term “linker” or “oligomeric backbone” refers to a chain of at least 10 contiguous atoms. In certain embodiments, the linker contains no more than 20 non-hydrogen atoms. The terms linker and oligomeric backbone can be used interchangeably. In some embodiments, the linker contains no more than 40 non-hydrogen atoms. In some embodiments, the linker contains no more than 60 non-hydrogen atoms. In certain embodiments, the linker contains atoms chosen from C, H, N, O, and S. In some embodiments, every non-hydrogen atom is chemically bonded either to 2 neighboring atoms in the linker, or one neighboring atom in the linker and a terminus of the linker. In some embodiments, the linker forms an amide bond with at least one of the two other groups to which it is attached. In certain embodiments, the linker forms an ester or ether bond with at least one of the two other groups to which it is attached. In some embodiments, the linker forms a thioester or thioether bond with at least one of the two other groups to which it is attached. In some embodiments, the linker forms a direct carbon-carbon bond with at least one of the two other groups to which it is attached. In some embodiments, the linker forms an amine or amide bond with at least one of the two other groups to which it is attached. In some embodiments, the linker comprises —(CH₂OCH₂)— units. In some embodiments, the linker comprises —(CH(CH₃)OCH₂)— units. In some embodiments, the linker comprises —(CH₂NR_{N}CH₂) units, for R_{N}═C₁₋₄alkyl. In some embodiments, the linker comprises an arylene, cycloalkylene, or heterocycloalkylene moiety.

The term “spacer” refers to a chain of at least 5 contiguous atoms. In some embodiments, the spacer contains no more than 10 non-hydrogen atoms. In some embodiments, the spacer contains atoms chosen from C, H, N, O, and S. In some embodiments, the spacer forms amide bonds with the two other groups to which it is attached. In certain embodiments, the spacer comprises —(CH₂OCH₂)— units. In some embodiments, the spacer comprises —(CH₂NR_{N}CH₂)— units, for R_{N}═C₁₋₄alkyl. In some embodiments, the spacer contains at least one positive charge at physiological pH.

The term “turn component” refers to a chain of about 4 to 10 contiguous atoms. In some embodiments, the turn component contains atoms chosen from C, H, N, O, and S. In some embodiments, the turn component forms amide bonds with the two other groups to which it is attached. In some embodiments, the turn component contains at least one positive charge at physiological pH.

The terms “nucleic acid and “nucleotide” refer to ribonucleotide and deoxyribonucleotide, and analogs thereof, well known in the art.

The term “oligonucleotide sequence” refers to a plurality of nucleic acids having a defined sequence and length (e.g., 2, 3, 4, 5, 6, or even more nucleotides). The term “oligonucleotide repeat sequence” refers to a contiguous expansion of oligonucleotide sequences.

The term “transcription,” well known in the art, refers to the synthesis of RNA (i.e., ribonucleic acid) by DNA-directed RNA polymerase. The term “modulate transcription” refers to a change in transcriptional level which can be measured by methods well known in the art, for example, assay of mRNA, the product of transcription. In certain embodiments, modulation is an increase in transcription. In other embodiments, modulation is a decrease in transcription

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 8 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amide,” as used herein, alone in combination, refers to —C(O)NRR′, wherein R and R′ are independently chosen from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted. Amides may be formed by direct condensation of carboxylic acids with amines, or by using acid chlorides. In addition, coupling reagents are known in the art, including carbodiimide-based compounds such as DCC and EDCI.

The term “amino,” as used herein, alone or in combination, refers to —NRR′, wherein R and R′ are independently chosen from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl. The term “arylene” embraces aromatic groups such as phenylene, naphthylene, anthracenylene, and phenanthrylene.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical, dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or combinations thereof, fully saturated or containing from 1 to 3 degrees of unsaturation, consisting of the stated number of carbon atoms and from one to three heteroatoms chosen from N, O, and S, and wherein the N and S atoms may optionally be oxidized and the N heteroatom may optionally be quaternized. The heteroatom(s) may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom chosen from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently chosen from nitrogen, oxygen, and sulfur. In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include tetrhydroisoquinoline, aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds or molecules of any one of the formulas disclosed herein.

The term “isocyanato” refers to a —NCO group.

The term “isothiocyanato” refers to a —NCS group.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “absent,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: alkyl, alkenyl, alkynyl, alkanoyl, heteroalkyl, heterocycloalkyl, haloalkyl, haloalkenyl, haloalkynyl, perhaloalkyl, perhaloalkoxy, cycloalkyl, phenyl, aryl, aryloxy, alkoxy, haloalkoxy, oxo, acyloxy, carbonyl, carboxyl, alkylcarbonyl, carboxyester, carboxamido, cyano, hydrogen, halogen, hydroxy, amino, lower alkylamino, arylamino, amido, nitro, thiol, alkylthio, haloalkylthio, perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, carbamate, and urea. Where structurally feasible, two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with”.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁—C)alkyl, nitro, 0-carbamyl, N-carbamyl, 0-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)— may be attached to the parent moiety at either the carbon or the nitrogen.

Asymmetric centers exist in the compounds or molecules disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and 1-isomers, and mixtures thereof. Individual stereoisomers of compounds or molecules can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds or molecules of particular stereochemistry are either commercially available or can be made and resolved by techniques known in the art. Additionally, the compounds or molecules disclosed herein may exist as geometric isomers. The present disclosure includes all cis, trans, syn, anti, entgegen (E), and zusammen (Z) isomers as well as the appropriate mixtures thereof. Additionally, compounds or molecules may exist as tautomers; all tautomeric isomers are provided by this disclosure. Additionally, the compounds or molecules disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the amount of active ingredients used in the treatment of a disease or disorder or on the effecting of a clinical endpoint.

The term “therapeutically acceptable” refers to those compounds or molecules (or salts, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “contacting” refers to bringing the compound (e.g. a transcription molecular molecule of the present disclosure) into proximity of the desired target gene. The contacting may result in the binding to or result in a conformational change of the target moiety.

The compounds or molecules disclosed herein can exist as therapeutically acceptable salts. The present disclosure includes compounds or molecules listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound or molecule in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

Basic addition salts can be prepared during the final isolation and purification of the compounds or molecules by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, and piperazine.

Other carrier materials and modes of administration known in the pharmaceutical art may also be used. Pharmaceutical compositions of the disclosure may be prepared by any of the well-known techniques of pharmacy, such as effective formulation and administration procedures. Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

The amount of active ingredient that may be combined with the carrier materials to produce a single dosage form will vary depending upon the host treated and the particular mode of administration.

The compounds or molecules can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. In addition, the route of administration may vary depending on the condition and its severity. The above considerations concerning effective formulations and administration procedures are well known in the art and are described in standard textbooks.

Included in the present disclosure are salts, particularly pharmaceutically acceptable salts, of the compounds described herein. The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

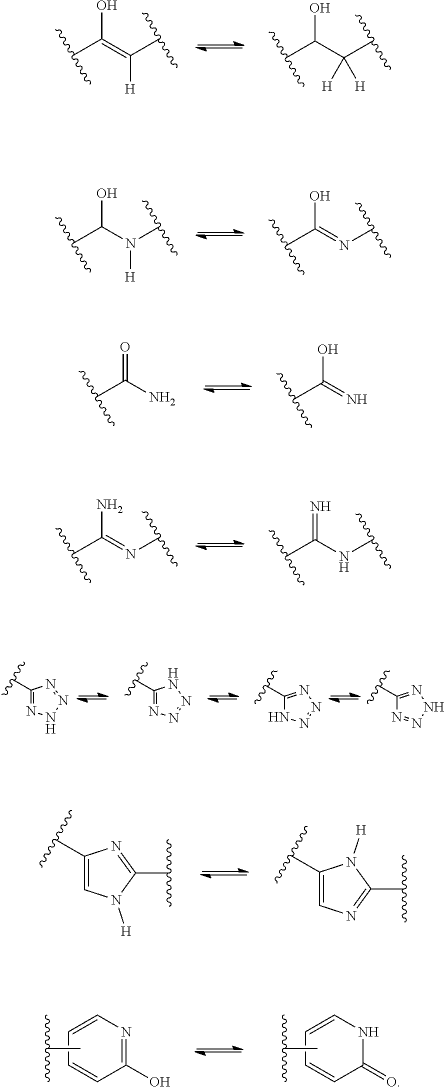

The compounds disclosed herein, in some embodiments, are used in different enriched isotopic forms, e.g., enriched in the content of ²H, ³H, ¹¹C, ³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ^{14F}, ^{15F}, ^{16F}, ^{17F}, ^{18F}, ³³S, ³⁴S, ³⁵S, ³⁶S ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention. In some embodiments, where isotopic variations are illustrated, the remaining atoms of the compound may optionally contain unnatural portions of atomic isotopes.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

The compounds described herein may in some cases exist as diastereomers, enantiomers, or other stereoisomeric forms. Where absolute stereochemistry is not specified, the compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will be known to those skilled in the art.

Compound Synthesis

Compounds of the present disclosure can be prepared using methods illustrated in general synthetic schemes and experimental procedures detailed below. General synthetic schemes and experimental procedures are presented for purposes of illustration and are not intended to be limiting. Starting materials used to prepare compounds of the present disclosure are commercially available or can be prepared using routine methods known in the art.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

The following examples are intended to illustrate but not limit the disclosed embodiments. Scheme A describes the steps involved for preparing the polyamide, attaching the polyamide to the oligomeric backbone, and then attaching the ligand to the other end of the oligomeric backbone. The transcription modulator molecule such as those listed in Table 4 can be prepared using the synthesis.

### List of Abbreviation

Ac₂O=acetic anhydride; AcCl=acetyl chloride; AcOH=acetic acid; AIBN=azobisisobutyronitrile; aq.=aqueous; Bu₃SnH=tributyltin hydride; CD₃OD=deuterated methanol; CDCl₃=deuterated chloroform; CDI=1,1′-Carbonyldiimidazole; DBU=1,8-diazabicyclo[5.4.0]undec-7-ene; DCM=dichloromethane; DEAD=diethyl azodicarboxylate; DIBAL-H=di-iso-butyl aluminium hydride; DIEA=DIPEA=N,N-diisopropylethylamine; DMAP=4-dimethylaminopyridine; DMF=N,N-dimethylformamide; DMSO-d₆=deuterated dimethyl sulfoxide; DMSO=dimethyl sulfoxide; DPPA=diphenylphosphoryl azide; EDC.HCl=EDCI·HCl=1-ethyl-3-(3-dimethylaminopropyl)carbodiimide hydrochloride; Et₂O=diethyl ether; EtOAc=ethyl acetate; EtOH=ethanol; h=hour; HATU=2-(1H-7-azabenzotriazol-1-yl)-1,1,3,3-tetramethyl uronium hexafluorophosphate methanaminium; HMDS=hexamethyldisilazane; HOBT=1-hydroxybenzotriazole; i-PrOH=isopropanol; LAH=lithium aluminium hydride; LiHMDS=Lithium bis(trimethylsilyl)amide; MeCN=acetonitrile; MeOH=methanol; MP-carbonate resin=macroporous triethylammonium methylpolystyrene carbonate resin; MsC1=mesyl chloride; MTBE=methyl tertiary butyl ether; MW=microwave irradiation; n-BuLi=n-butyllithium; NaHMDS=Sodium bis(trimethylsilyl)amide; NaOMe=sodium methoxide; NaOtBu=sodium t-butoxide; NBS=N-bromosuccinimide; NCS=N-chlorosuccinimide; NMP=N-Methyl-2-pyrrolidone; Pd(Ph₃)₄=tetrakis(triphenylphosphine)palladium(0); Pd₂(dba)₃=tris(dibenzylideneacetone)dipalladium(0); PdCl₂(PPh₃)₂=bis(triphenylphosphine)palladium(II) dichloride; PG=protecting group; prep-HPLC=preparative high-performance liquid chromatography; PyBop=(benzotriazol-1-yloxy)-tripyrrolidinophosphonium hexafluorophosphate; Pyr=pyridine; RT=room temperature; RuPhos=2-dicyclohexylphosphino-2′,6′-diisopropoxybiphenyl; sat.=saturated; ss=saturated solution; t-BuOH=tert-butanol; T3P=Propylphosphonic Anhydride; TBS=TBDMS=tert-butyldimethylsilyl; TBSC1=TBDMSCl=tert-butyldimethylchlorosilane; TEA=Et₃N=triethylamine; TFA=trifluoroacetic acid; TFAA=trifluoroacetic anhydride; THF=tetrahydrofuran; Tol=toluene; TsC1=tosyl chloride; XPhos=2-dicyclohexylphosphino-2′,4′,6′-triisopropylbiphenyl.

### Synthesis of Representative Polyamides

### Example 1. Synthesis of DNA-Binding Moiety PA-004

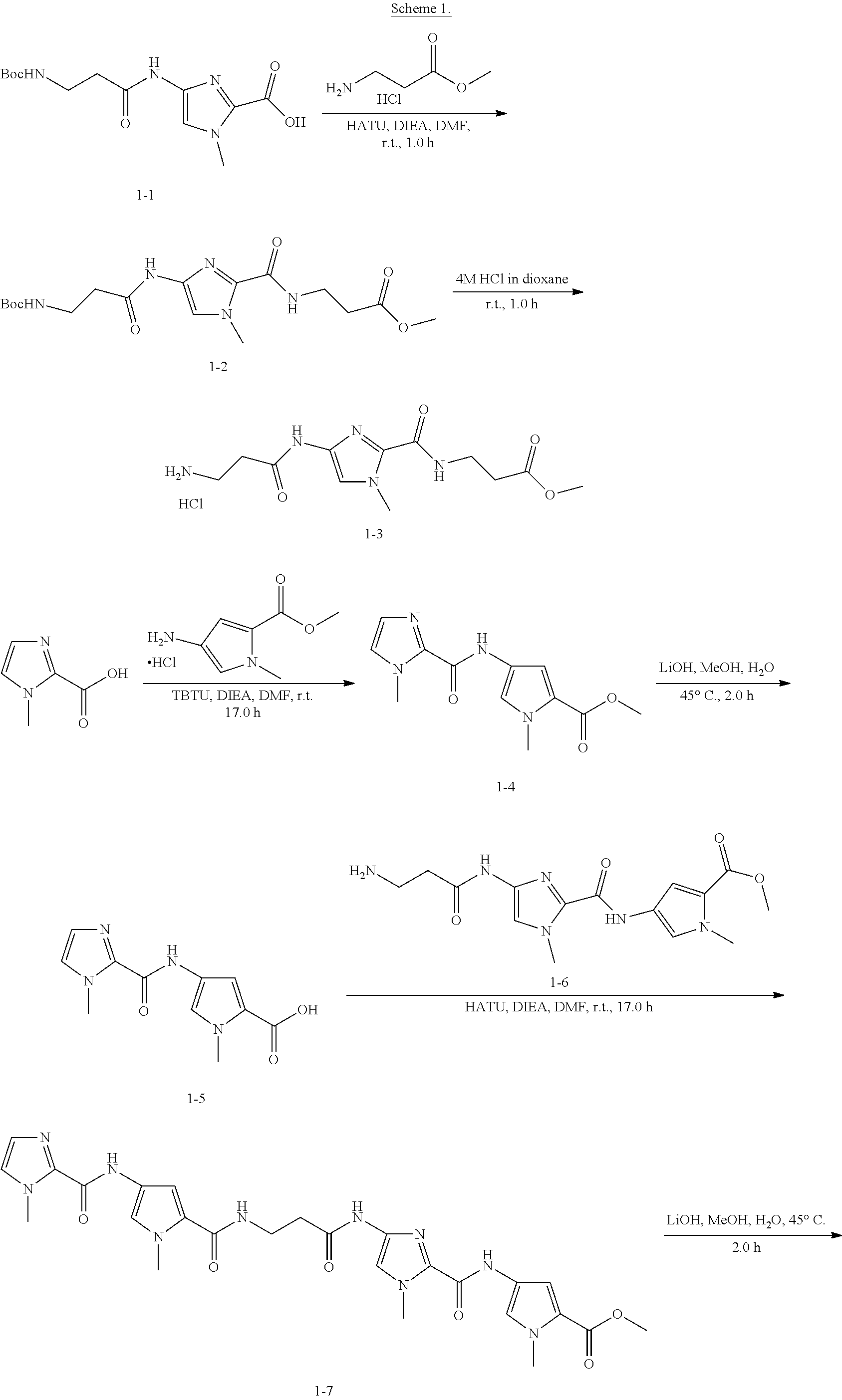

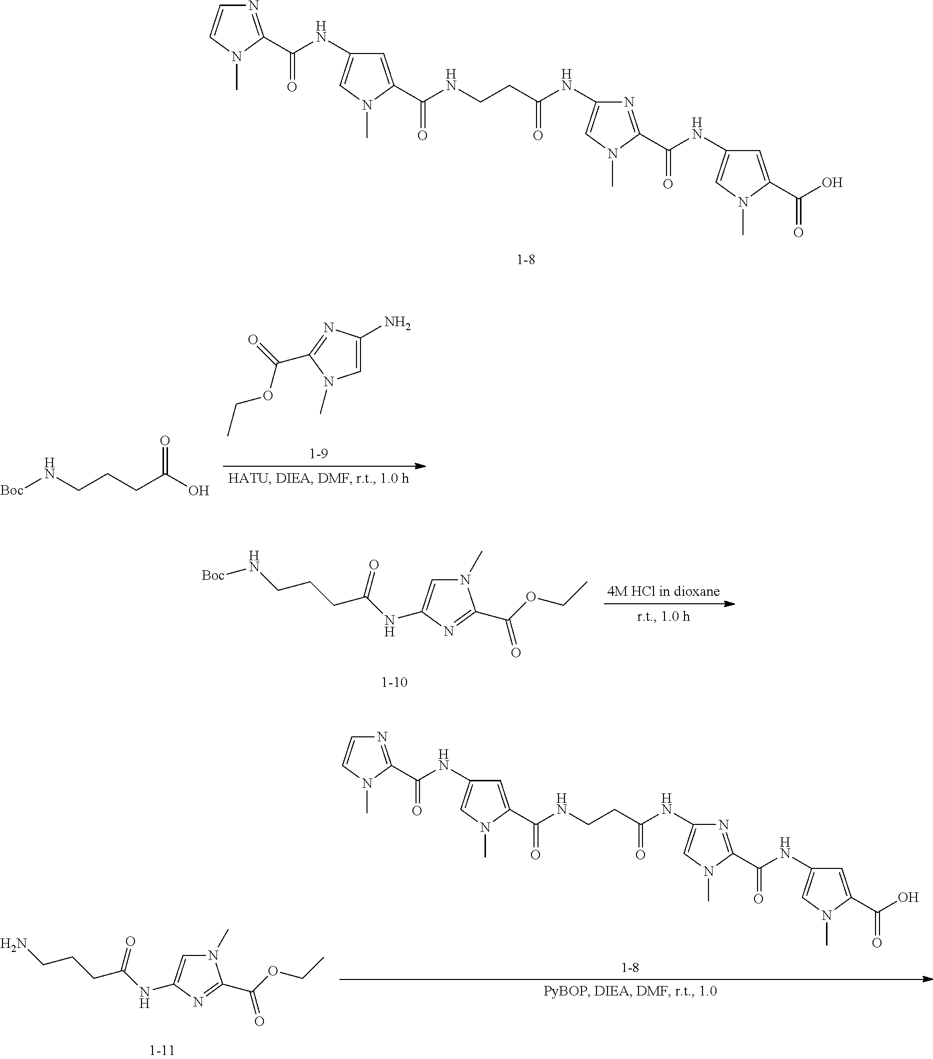 [SMILES: Cn1cc(NC(=O)c2nc(NC(=O)CCNC(=O)c3cc(NC(=O)c4nccn4C)cn3C)cn2C)cc1C(=O)O ; CCOC(=O)c1nc(N)cn1C ; CC(C)(C)OC(=O)NCCCC(=O)O ; CCOC(=O)c1nc(NC(=O)CCCNC(=O)OC(C)(C)C)cn1C ; CCOC(=O)c1nc(NC(=O)CCCN)cn1C ; Cn1cc(NC(=O)c2nc(NC(=O)CCNC(=O)c3cc(NC(=O)c4nccn4C)cn3C)cn2C)cc1C(=O)O]

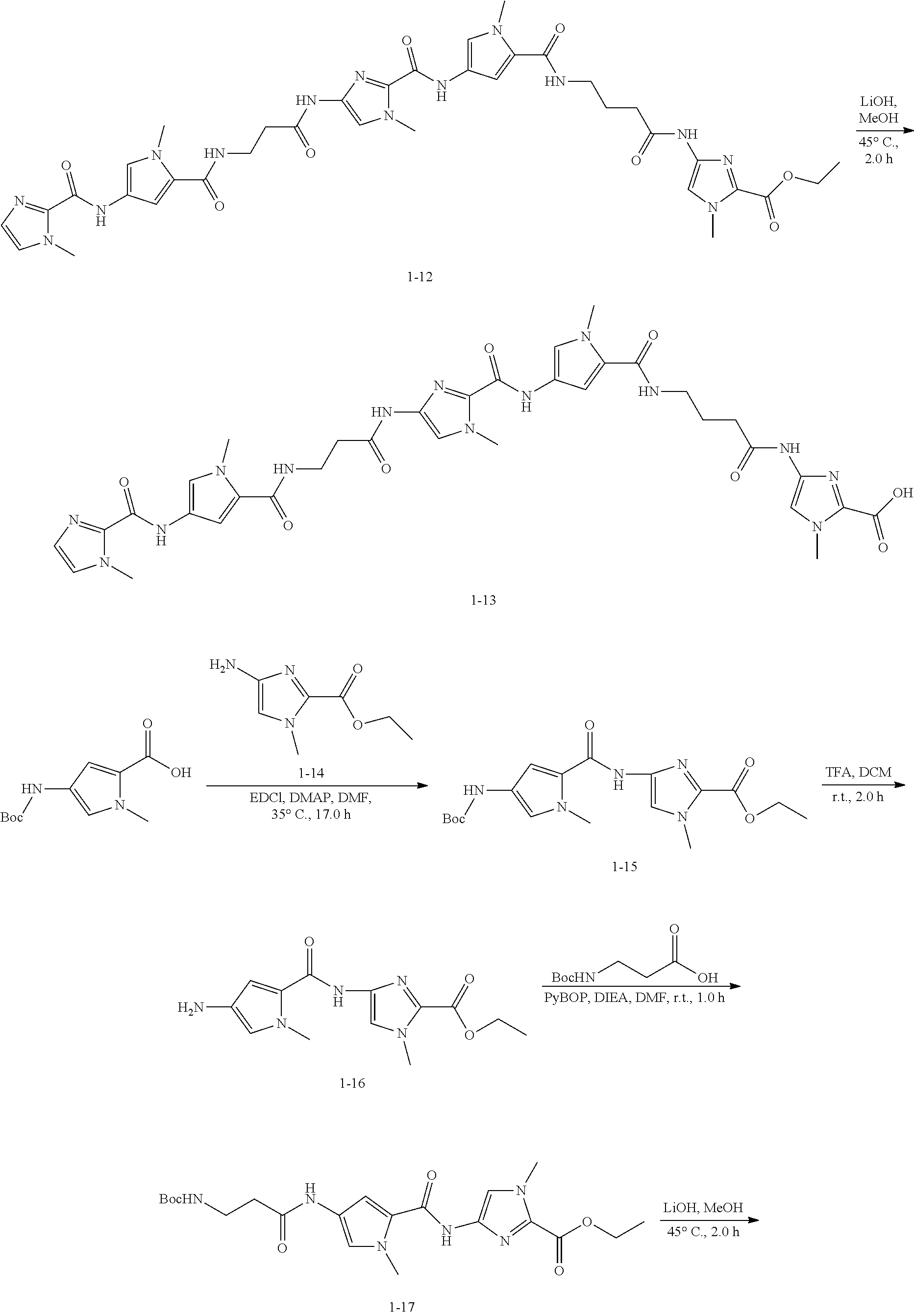

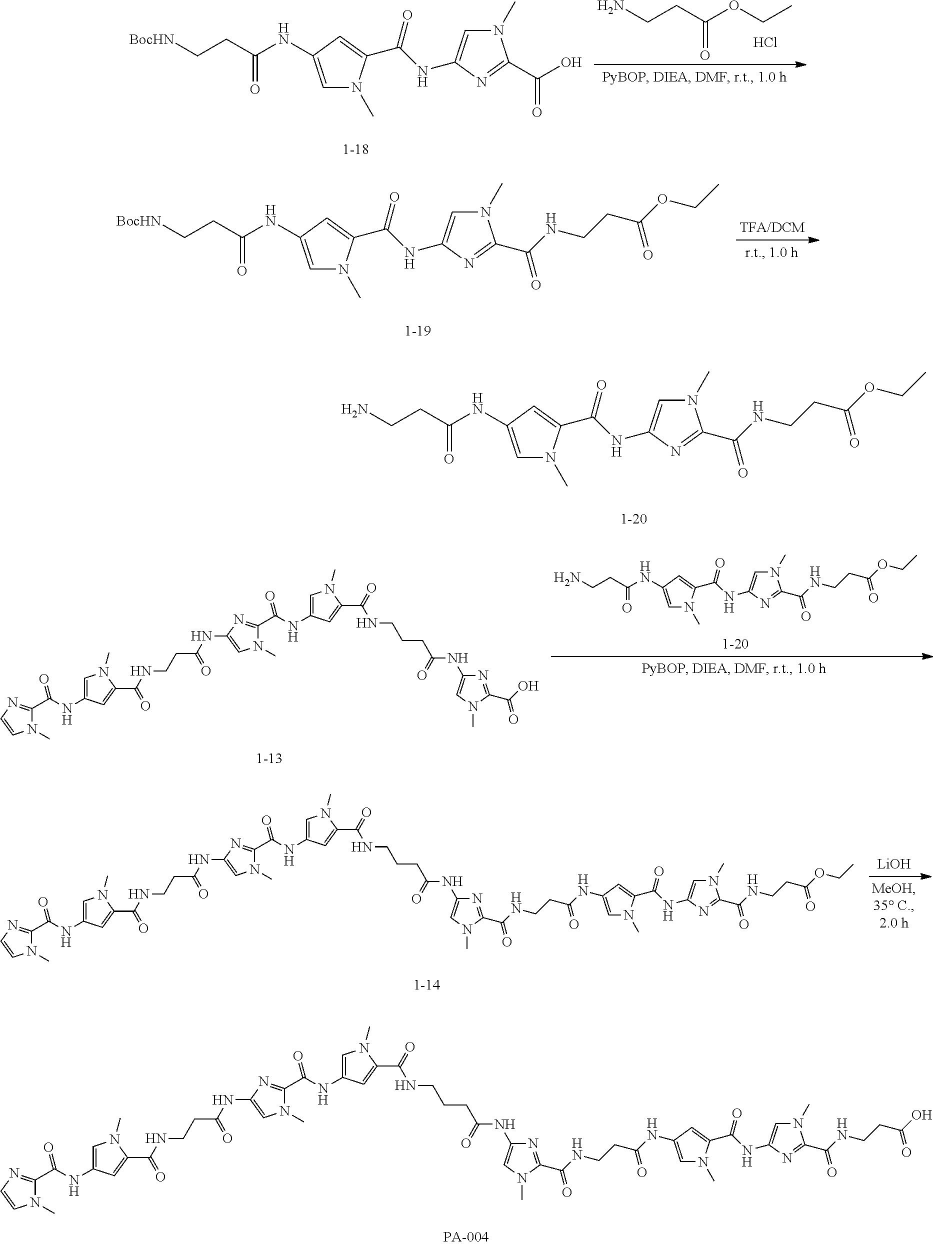

### Step 1: Synthesis of methyl 3-[(4-[3-[(tert-butoxycarbonyl)amino]propanamido]-1-methylimidazol-2-yl)formamido]propanoate

Into a 1000 ml flask was added 4-[3-[(tert-butoxycarbonyl)amino]propanamido]-1-methylimidazole-2-carboxylic acid (11.00 g, 35.22 mmol, 1.00 equiv), DMF (300.00 mL), the mixture was cooled to 0° C., then HATU (20.09 g, 52.83 mmol, 1.50 equiv), DIEA (18.21 g, 140.88 mmol, 4.00 equiv) was added dropwise, the mixture was stirred for 10 mins, methyl 3-aminopropanoate (3.63 g, 35.22 mmol, 1.00 equiv) was added in portions. The reaction was stirred at room temperature for 1.0 h. The reaction mixture was poured into water/ice (600 mL), the solid was filtered out and dried under vacuum. The aqueous phase was extracted by EA (3×200 mL), the organic phases were combined and washed by H₂O (1×200 mL) and NaCl (1×200 mL), dried over anhydrous Na₂SO₄. After filtration, the filtrate was concentrated under reduced pressure. The residue was purified by silica gel column, eluted with pure EA. The fractions were combined and concentrated. Methyl 3-[(4-[3-[(tert-butoxycarbonyl)amino]propanamido]-1-methylimidazol-2-yl)formamido]propanoate (13.00 g, 87.95%) was obtained as a yellow solid. LC/MS: mass calcd. For C₁₇H₂₇N₅O₆: 397.20, found: 398.20 [M+H]⁺.

### Step 2: Synthesis of methyl 3-[[4-(3-aminopropanamido)-1-methylimidazol-2-yl]formamido]propanoate hydrochloride

The procedure was the same as methyl 4-[4-(3-aminopropanamido)-1-methylimidazole-2-amido]-1-methylpyrrole-2-carboxylate hydrochloride, but the reaction time was 1.0 h. 11.00 g of methyl 3-[(4-[3-[(tert-butoxycarbonyl)amino]propanamido]-1-methylimidazol-2-yl)formamido]propanoate was used, 11.00 g crude of desired product was obtained as yellow oil. LC/MS: mass calcd. For C₁₂H₁₉N₅O₄: 297.14, found: 298.20 [M+H]⁺.

### Step 3: Synthesis of Methyl 1-methyl-4-(1-methylimidazole-2-amido)pyrrole-2-carboxylate

To a stirred solution of 1-methylimidazole-2-carboxylic acid (10.00 g, 79.29 mmol, 7.00 equiv) in DMF (150.00 mL) was added TBTU (38.19 g, 118.94 mmol, 1.50 equiv), methyl 4-amino-1-methylpyrrole-2-carboxylate hydrochloride (16.63 g, 87.24 mmol, 1.10 equiv) and DIEA (30.74 g, 237.88 mmol, 3.00 equiv) in portions at 0° C. The resulting mixture was stirred for 17.0 h at room temperature. The reaction was poured into water/Ice (450 mL). The precipitated solids were collected by filtration and washed with H₂O (3×50 mL), dried under vacuum. Methyl 1-methyl-4-(1-methylimidazole-2-amido)pyrrole-2-carboxylate (16.5 g, 78.37%) was obtained as a white solid. LC/MS: mass calcd. For C₁₂H₁₄N₄O₃: 262.11, found: 263.15 [M+H]⁺.

### Step 4: Synthesis of 1-methyl-4-(1-methylimidazole-2-amido)pyrrole-2-carboxylic acid

The procedure was the same as 4-[3-[(tert-butoxycarbonyl)amino]propanamido]-1-methylimidazole-2-carboxylic acid. 16.50 g of methyl 1-methyl-4-(1-methylimidazole-2-amido)pyrrole-2-carboxylate was used, 12.00 g of 1-methyl-4-(1-methylimidazole-2-amido)pyrrole-2-carboxylic acid (76.84% yield) was obtained as white solid. LC/MS: mass calcd. For C₁₁H₁₂N₄O₃: 248.09, found: 249.10 [M+H]⁺.

### Step 5: Synthesis of methyl 1-methyl-4-[1-methyl-4-(3-[[1-methyl-4-(1-methylimidazole-2-amido)pyrrol-2-yl]formamido]propanamido)imidazole-2-amido]pyrrole-2-carboxylate

The procedure was the same as ethyl 3-[(4-[3-[(tert-butoxycarbonyl)amino]propanamido]-1-methylimidazol-2-yl)formamido]propanoate. 9.00 g of 1-methyl-4-(1-methylimidazole-2-amido)pyrrole-2-carboxylic acid was used, 14.00 g of desired product (63.54% yield) was obtained yellow solid. LC/MS: mass calcd. For C₂₆H₃₀N₁₀O₆: 578.23, found: 579.10 [M+H]⁺.

### Step 6: Synthesis of 1-methyl-4-[1-methyl-4-(3-[[1-methyl-4-(1-methylimidazole-2-amido)pyrrol-2-yl]formamido]propanamido)imidazole-2-affordamido]pyrrole-2-carboxylic acid

The procedure was the same as 4-[3-[(Tert-butoxycarbonyl)amino]propanamido]-1-methylimidazole-2-carboxylic acid. 14.00 g of methyl 1-methyl-4-[1-methyl-4-(3-[[1-methyl-4-(1-methylimidazole-2-amido) pyrrol-2-yl]formamido]propanamido)imidazole-2-amido]pyrrole-2-yl]formamidocarboxylate was used, 12.00 g of desired product (81.49% yield) was obtained as yellow solid. LC/MS: mass calcd. For C₂₅H₂₈N₁₀O₆: 564.22, found: 565.15[M+H]⁺.

### Step 7: Synthesis of ethyl 4-{4-[(tert-butoxycarbonyl)amino]butanamido}-1-methylimidazole-2-carboxylate

The procedure was the same as ethyl 4-[3-[(tert-butoxycarbonyl)amino]propanamido]-1-methylimidazole-2-carboxylate. 7.80 g of 4-[(tert-butoxycarbonyl)amino]butanoic acid was obtained, 11.00 g of desired product was obtained as a pink solid (80.70% yield). LC/MS: mass calcd. For C₁₆H₂₆N₄O₅: 354.19, found: 355.15[M+H]⁺.

### Step 8: Synthesis of ethyl 4-(4-aminobutanamido)-1-methylimidazole-2-carboxylate

The procedure was the same as methyl 4-[4-(3-aminopropanamido)-1-methylimidazole-2-amido]-1-methylpyrrole-2-carboxylate hydrochloride. 9.40 g of ethyl 4-{4-[(tert-butoxycarbonyl)amino]butanamido}-1-methylimidazole-2-carboxylate was used, 6.20 g of desired product was obtained as a white solid (90.89% yield). LCMS: mass calcd. For C₁₁H₁₈N₄O₃: 254.14, found: 255.15[M+H]⁺.

### Step 9: Synthesis of ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate

To a stirred solution of 1-methyl-4-[1-methyl-4-(3-{[1-methyl-4-(1-methylimidazole-2-amido)pyrrol-2-yl]formamido}propanamido)imidazole-2-amido]pyrrole-2-carboxylic acid (18.20 g, 32.24 mmol, 1.00 equiv) in DMF (250.00 mL) was added DIEA (12.50 g, 96.71 mmol, 3.00 equiv), ethyl 4-(4-aminobutanamido)-1-methylimidazole-2-carboxylate (9.02 g, 35.46 mmol, 1.10 equiv) and PyBOP (20.13 g, 38.68 mmol, 1.20 equiv) at 0° C. The resulting mixture was stirred for 1.0 h at room temperature. The reaction was poured into ice/water (800 mL). The precipitated solids were collected by filtration and washed with H₂O (3×200 mL), dried under vacuum. 24.70 g of ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate was obtained as a yellow solid (95.74% yield). LC/MS: mass calcd. For C₃₆H₄₄N₁₄O₈: 800.35, found: 801.30[M+H]⁺.

### Step 10: Synthesis of 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylic acid

The procedure was the same as 4-[3-[(tert-butoxycarbonyl)amino]propanamido]-1-methylimidazole-2-carboxylic acid. 24.00 g of ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate was used, 23.10 g of desired product was obtained as a yellow solid (99.36% yield). LC/MS: mass calcd. For C₃₄H₄₀N₁₄O₈: 772.32, found: 773.30[M+H]⁺.

### Step 11: Synthesis of ethyl 4-[4-[(tert-butoxycarbonyl)amino]-1-methylpyrrole-2-amido]-1-methylimidazole-2-carboxylate

To a stirred solution of 4-[(tert-butoxycarbonyl)amino]-1-methylpyrrole-2-carboxylic acid (11.50 g, 47.87 mmol, 1.00 equiv) in DMF (200.00 mL) was added EDCI (22.94 g, 119.66 mmol, 2.50 equiv), ethyl 4-amino-1-methylimidazole-2-carboxylate (8.10 g, 47.87 mmol, 1.00 equiv) and DMAP (14.62 g, 119.66 mmol, 2.50 equiv) at 0° C. The resulting mixture was stirred for 17.0 h at 35° C. After reaction, the reaction was poured into 500 mL ice/water. The precipitated solids were collected by filtration and washed with water (3×50 mL), dried under vacuum. This resulted in ethyl 4-{4-[(tert-butoxycarbonyl)amino]-1-methylpyrrole-2-amido}-1-methylimidazole-2-carboxylate (16.00 g, 85.48% yield) as a light yellow solid. LC/MS: mass calcd. For C₁₈H₂₅N₅O₅: 391.19, found: 392.30 [M+H]⁺.

### Step 12: Synthesis of ethyl 4-(4-amino-1-methylpyrrole-2-amido)-1-methylimidazole-2-carboxylate

To a stirred solution of ethyl 4-{4-[(tert-butoxycarbonyl)amino]-1-methylpyrrole-2-amido}-1-methylimidazole-2-carboxylate (16.00 g, 40.88 mmol, 1.00 equiv) in DCM (135.00 mL) were added and TFA (45.00 mL) dropwise at room temperature. The resulting mixture was stirred for 2.0 h at room temperature. The resulting mixture was concentrated under vacuum. The residue brown oil was diluted with Et₂O (200 mL). The precipitated solids were collected by filtration and washed with Et₂O (2×100 mL). The resulting solid was dried under vacuum. This resulted in ethyl 4-(4-amino-1-methylpyrrole-2-amido)-1-methylimidazole-2-carboxylate (16.00 g, crude) as a brown solid. LC/MS: mass calcd. For C₁₃H₁₇N₅O₃: 291.13, found: 292.15[M+H]⁺.

### Step 13: Synthesis of ethyl 4-(4-{3-[(tert-butoxycarbonyl)amino]propanamido}-1-methylpyrrole-2-amido)-1-methylimidazole-2-carboxylate

A solution of ethyl 4-(4-amino-1-methylpyrrole-2-amido)-1-methylimidazole-2-carboxylate (12.00 g, 41.19 mmol, 1.00 equiv) and 3-[(tert-butoxycarbonyl)amino]propanoic acid (7.50 g, 39.64 mmol, 0.96 equiv), PyBOP (22.00 g, 42.28 mmol, 1.03 equiv), DIEA (45.00 g, 348.18 mmol, 8.45 equiv) in DMF (120.00 mL) was stirred for 1.0 h at room temperature. The reaction was poured into ice water (400 mL), and the mixture was stirred for 15 min. The precipitated solids were collected by filtration and washed with water (3×150 mL) and dried under vacuum. The aqueous phase was extracted by EA (3×150 mL), the combined organic phases were combined and washed by H₂O (200 mL), dried over anhydrous Na₂SO₄. The solid was filtered out and the filtrate was concentrated. The residue was purified by silica gel column chromatography, eluted with PE/EA (1:8). This resulted in 17.00 g of ethyl 4-(4-{3-[(tert-butoxycarbonyl)amino]propanamido}-1-methylpyrrole-2-amido)-1-methylimidazole-2-carboxylate was obtained as a yellow solid (89.28% yield). LC/MS: mass calcd. For C₂₁H₃₀N₆O₆: 462.22, found: 463.35[M+H]⁺.

### Step 14: Synthesis of 4-(4-{3-[(tert-butoxycarbonyl)amino]propanamido}-1-methylpyrrole-2-amido)-1-methylimidazole-2-carboxylic acid

The procedure was the same as 4-[3-[(tert-butoxycarbonyl)amino]propanamido]-1-methylimidazole-2-carboxylic acid. 12.00 g of ethyl 4-(4-{3-[(tert-butoxycarbonyl)amino]propanamido}-1-methylpyrrole-2-amido)-1-methylimidazole-2-carboxylate was used, 10.00 g of the desired product was obtained as a white solid (88.81% yield). LC/MS: mass calcd. For C₁₉H₂₆N₆O₆: 434.19, found: 435.25 [M+H]⁺.

### Step 15: Synthesis of ethyl 3-{[4-(4-{3-[(tert-butoxycarbonyl)amino]propanamido}-1-methylpyrrole-2-amido)-1-methylimidazol-2-yl]formamido}propanoate

A solution of 4-(4-{3-[(tert-butoxycarbonyl)amino]propanamido}-1-methylpyrrole-2-amido)-1-methylimidazole-2-carboxylic acid (10.00 g, 23.02 mmol, 1.00 equiv) and β-alanine ethyl ester hydrochloride (4.90 g, 31.90 mmol, 1.39 equiv), PyBOP (12.50 g, 24.02 mmol, 1.04 equiv), DIEA (9.00 g, 69.64 mmol, 3.03 equiv) in DMF (120.00 mL) was stirred for 1.0 h at room temperature. The reaction was quenched by the addition of water (500 mL) at room temperature. The resulting mixture was extracted with EtOAc (3×400 mL). The combined organic layers were washed with brine (3×200 mL), dried over anhydrous Na₂SO₄. After filtration, the filtrate was concentrated under reduced pressure. The residue was purified by silica gel column chromatography, eluted with PE/EA (1:8) to afford ethyl 3-{[4-(4-{3-[(tert-butoxycarbonyl)amino]propanamido}-1-methylpyrrole-2-amido)-1-methylimidazol-2-yl]formamido}propanoate (12.00 g, 93.80%) as a yellow solid. LC/MS: mass calcd. For C₂₄H₃₅N₇O₇: 533.26, found: 534.30[M+H]⁺.

### Step 16: Synthesis of ethyl 3-({4-[4-(3-aminopropanamido)-1-methylpyrrole-2-amido]-1-methylimidazol-2-yl}formamido)propanoate

The procedure was the same as ethyl 4-(4-amino-1-methylpyrrole-2-amido)-1-methylimidazole-2-carboxylate. Ethyl 3-{[4-(4-{3-[(tert-butoxycarbonyl)amino]propanamido}-1-methylpyrrole-2-amido)-1-methylimidazol-2-yl]formamido}propanoate was used to obtain 12.00 g crude of the desired product was obtained as a white solid. LC/MS: mass calcd. For C₁₉H₂₇N₇O₅: 433.21, found: 434.25[M+H]⁺.

### Step 17: Synthesis of ethyl 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate. 10.00 g of 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylic acid was used, 13.60 g of the desired product was obtained as a yellow solid (88.61% yield). Some pure product was obtained as a light yellow solid after purification by Prep-HPLC. HRMS: mass calcd. For C₅₃H₆₅N₂₁O₁₂: 1187.5122, found: 1188.5153[M+H]⁺.

### Step 18: Synthesis of 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid (PA-004)

The procedure was the same as 4-[3-[(tert-butoxycarbonyl)amino]propanamido]-1-methylimidazole-2-carboxylic acid, but the reaction temperature was 35° C. 10.60 g of ethyl 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoate was used, 10.00 g crude of the desired product was obtained as a yellow solid. LC/MS: mass calcd. For C₅₁H₆N₂₁O₁₂: 1159.48, found: 581.25[M/2+H]⁺.

### Example 2. Synthesis of DNA-Binding Moiety (PA-023)

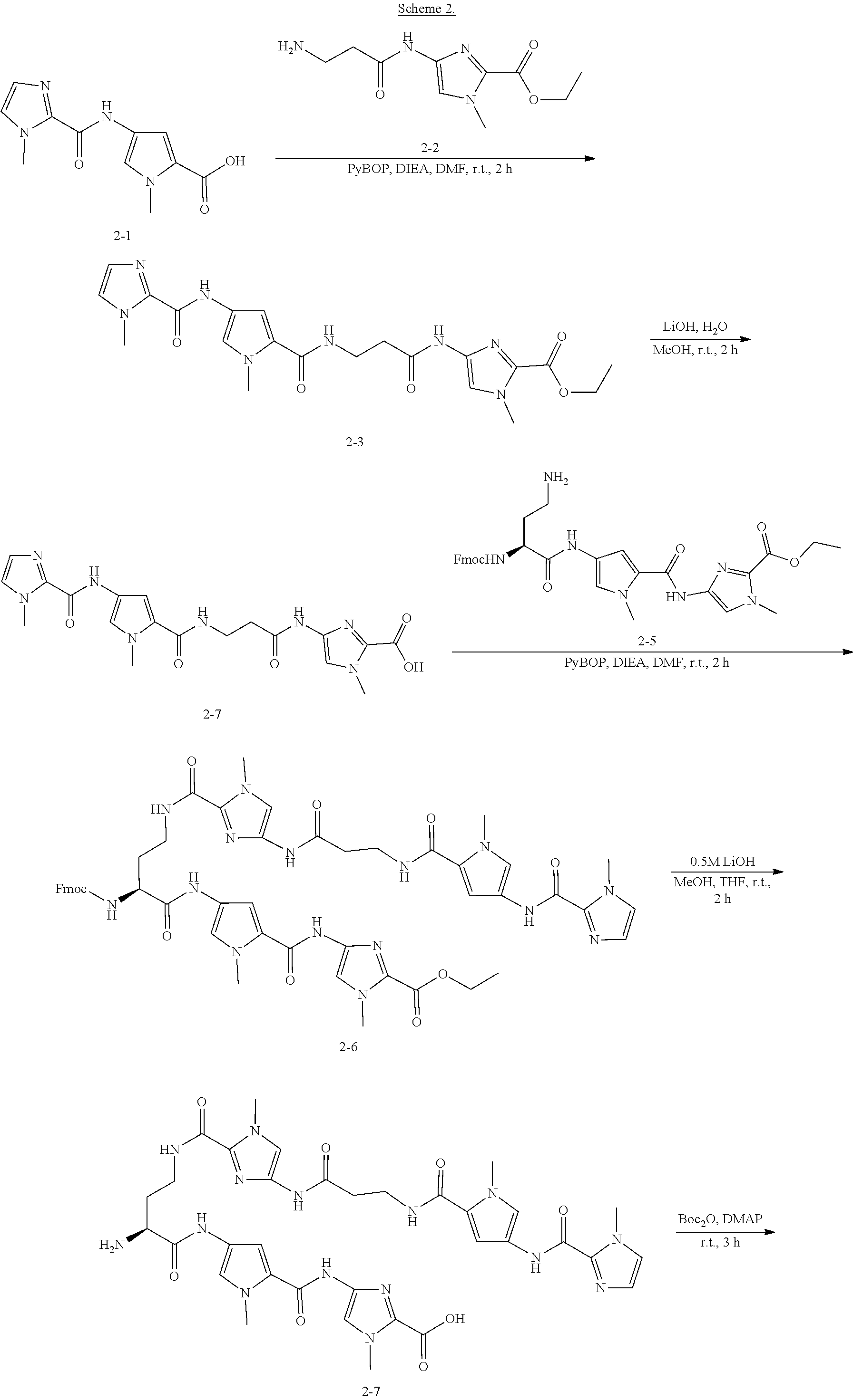

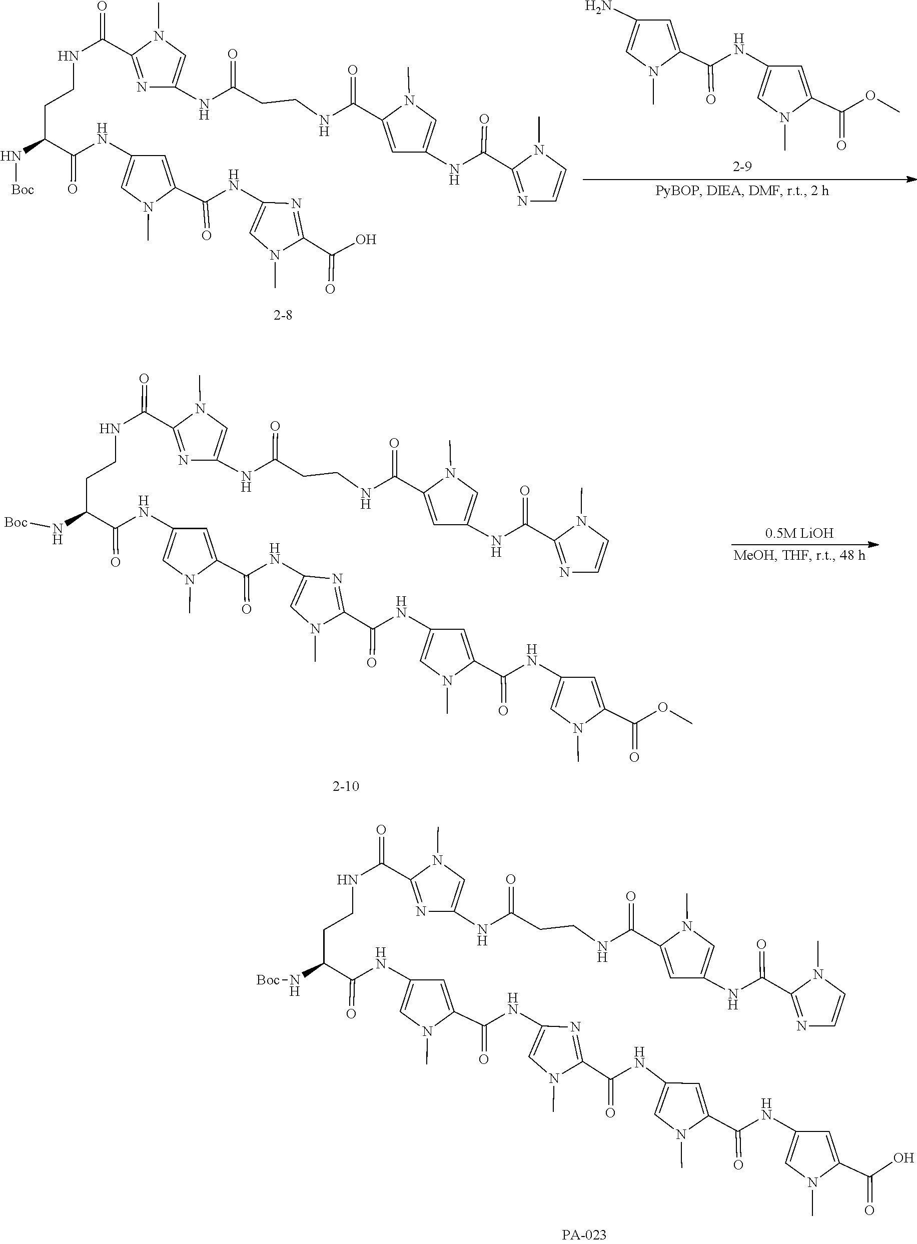

### Step 1: Synthesis of ethyl 1-methyl-4-(3-{[1-methyl-4-(1-methylimidazole-2-amido)pyrrol-2-yl]formamido}propanamido)imidazole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate, but the reaction time was 2.0 h. 1.50 g of ethyl 4-(3-aminopropanamido)-1-methylimidazole-2-carboxylate was used, 2.00 g of desired product was obtained as an off-white solid (68.09% yield). LC/MS: mass calcd. For C₂₁H₂₆N₈O₅: 470.20, found: 471.40 [M+H]⁺.

### Step 2: Synthesis of 1-methyl-4-(3-{[1-methyl-4-(1-methylimidazole-2-amido) pyrrol-2-yl]formamido}propanamido)imidazole-2-carboxylic acid

The procedure was the same as 4-[3-[(tert-butoxycarbonyl)amino]propanamido]-1-methylimidazole-2-carboxylic acid, but the reaction temperature was room temperature, the reaction time was 2.0 h. 2.00 g of ethyl 1-methyl-4-(3-{[1-methyl-4-(1-methylimidazole-2-amido)pyrrol-2-yl]formamido}propanamido)imidazole-2-carboxylate was used, 1.80 g of 1-methyl-4-(3-{[1-methyl-4-(1-methylimidazole-2-amido)pyrrol-2-yl]formamido}propanamido)imidazole-2-carboxylic acid was obtained as an off-white solid (95.71% yield). LC/MS: mass calcd. For C₁₉H₂₂N₈O₅: 442.17, found: 443.10 [M+H]⁺.

### Step 3: Synthesis of ethyl 4-{4-[(2S)-2-{[(9H-fluoren-9-ylmethoxy)carbonyl]amino}-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate, but the reaction time was 2.0 h. 1.60 g of ethyl 4-{4-[(2S)-4-amino-2-{[(9H-fluoren-9-ylmethoxy)carbonyl]amino}butanamido]-1-methylpyrrole-2-amido}-1-methylimidazole-2-carboxylate was used, 1.90 g of desired product was obtained as a light yellow solid (70.20% yield). LC/MS: mass calcd. For C₅H₅₅N₁₅O₁₀: 1037.43, found: 1038.45 [M+H]⁺.

### Step 4: Synthesis of 4-[4-(4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylic acid

A mixture of ethyl 4-{4-[(2S)-2-{[(9H-fluoren-9-ylmethoxy)carbonyl]amino}-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylate (1.90 g, 1.83 mmol, 1.00 equiv) and LiOH (0.22 g, 9.15 mmol, 5.00 equiv) in MeOH (5.00 mL), THF (15.00 mL) and H₂O (18.30 mL) was stirred for 2.0 h at room temperature. The resulting mixture was used in the next step directly without further purification. LC/MS: mass calcd. For C₃₄H₄₁N₁₅O₈: 787.33, found: 788.40 [M+H]⁺.

### Step 5: Synthesis of 4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylic acid

The mixture of 4-{4-[(2S)-2-amino-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylic acid (1.40 g, 1.78 mmol, 1.00 equiv) in MeOH/THF/H₂O (5.00 mL/15.00 mL/18.30 mL) was added di-tert-butyl dicarbonate (0.78 g, 3.55 mmol, 2.00 equiv) and DMAP (0.02 g, 0.18 mmol, 0.10 equiv). The reaction was stirred at room temperature for 3.0 h. The mixture was added with H₂O (30 mL). The mixture was filtered through a Celite pad, and the solid was washed with ethyl acetate (3×30 mL) to afford 4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylic acid (1.20 g, 76.05% yield) as a yellow solid. LC/MS: mass calcd. For C₃₉H₄₉N₁₅O₁₀: 887.38, found: 888.45 [M+H]⁺.

### Step 6: Synthesis of methyl 4-[4-(4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido) imidazole-2-amido]pyrrol-2-yl}formamido)butanamido]imidazole-2-carboxylate, but the reaction time was 2.0 h. 1.20 g of 4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylic acid was used, 1.10 g of the desired product was obtained as a yellow solid (71.01% yield). LC/MS: mass calcd. For C₅₂H₆₃N₁₉O₁₂: 1145.49, found: 1146.50 [M+H]⁺.

### Step 7: Synthesis of 4-[4-(4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylic acid (PA-023)

The procedure was the same as 4-[4-(4-{4-[(2S)-2-[(tert-butoxycarbonyl)amino]-4-[(1-methyl-4-{1-methyl-4-[1-methyl-4-(1-methylimidazole-2-amido)pyrrole-2-amido]pyrrole-2-amido}imidazol-2-yl)formamido]butanamido]-1-methylpyrrole-2-amido}-1-methylimidazole-2-amido)-1-methylpyrrole-2-amido]-1-methylpyrrole-2-carboxylic acid. 1.00 g of methyl 4-[4-(4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylate was used, 400.00 mg of the desired product was obtained as a white solid (39.16% yield). LC/MS: mass calcd. For C₅₁H₁N₁₉O₁₂: 1131.47, found: 1132.65 [M+H]⁺.

### Example 3. Synthesis of DNA-Binding Moiety (PA-040)

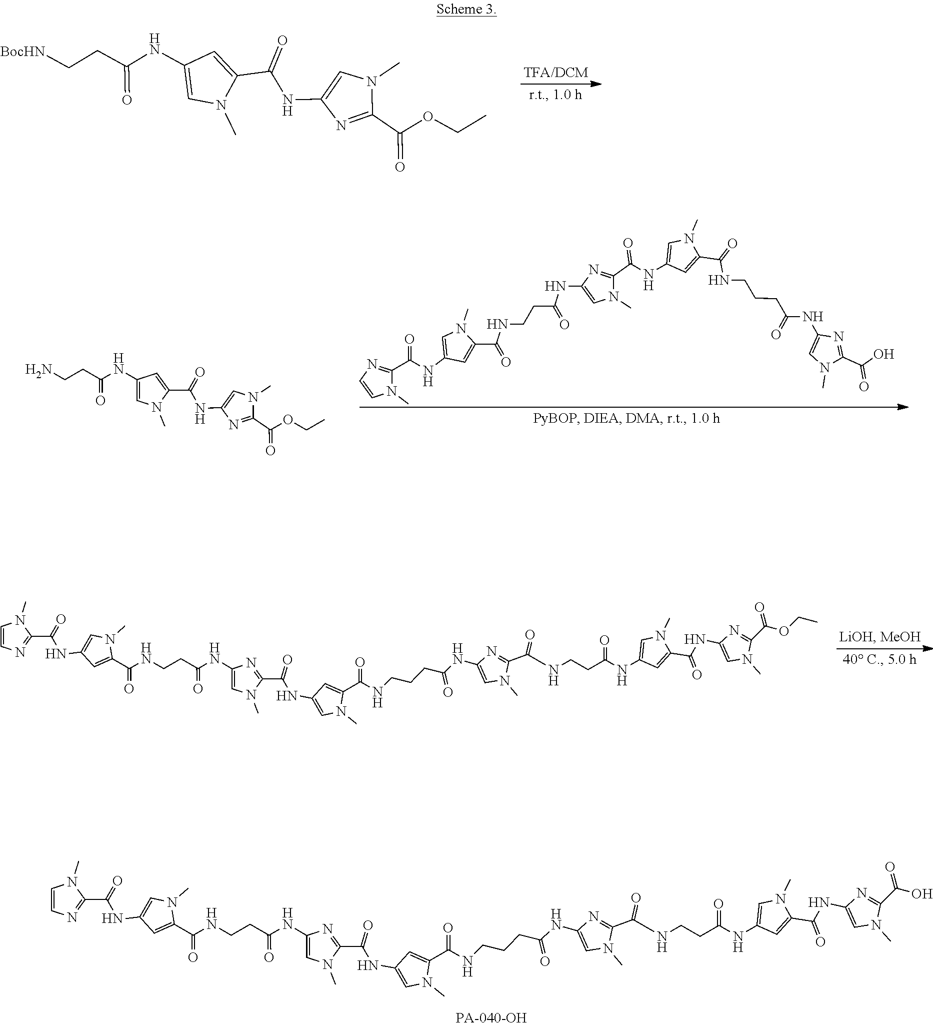 [SMILES: CCOC(=O)c1nc(NC(=O)c2cc(NC(=O)CCNC(=O)OC(C)(C)C)cn2C)cn1C ; CCOC(=O)c1nc(NC(=O)c2cc(NC(=O)CCN)cn2C)cn1C ; Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)O)n1 ; CCOC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C ; Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)O)n3)n(C)c2)n1]

### Step 1: Synthesis of ethyl 4-[4-(3-aminopropanamido)-1-methylpyrrole-2-amido]-1-methylimidazole-2-carboxylate

The procedure was the same as ethyl 4-(4-amino-1-methylpyrrole-2-amido)-1-methylimidazole-2-carboxylate (Intermediate 1-16, Example 1). 2.00 g of ethyl 4-(4-{3-[(tert-butoxycarbonyl)amino]propanamido}-1-methylpyrrole-2-amido)-1-methylimidazole-2-carboxylate was used, 2.00 g crude of the desired product was obtained as a white solid. LC/MS: mass calcd. For C₁₆H₂₂N₆O₄: 362.17, found: 363.25[M+H]⁺.

### Step 2: Synthesis of tert-butyl N-(3-[[3-(1,3-dioxoisoindol-2-yl)propyl](methyl) amino]propyl)-N-methylcarbamate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate (Int. 1-12), but the solvent was DMA. 3.00 g of 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylic acid was used, 4.30 g of the desired product was obtained as a yellow solid (96.84% yield). LC/MS: mass calcd. For C₅₀H₆₀N₂₀O₁₁: 1116.48, found: 1117.60[M+H]⁺.

### Step 3: Synthesis of 1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazole-2-carboxylic acid (PA-040-OH)

The procedure was the same as Example 1 (PA-004), but the reaction temperature was 40° C., the reaction time was 5.0 h. 4.20 g of ethyl 1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazole-2-carboxylate was used, 4.00 g of the desired product was obtained as a yellow solid (97.97% yield). LC/MS: mass calcd. For C₄₈H₅₆N₂₀O₁₁: 1088.44, found: 1089.55[M+H]⁺.

### Example 4. Synthesis of A-27

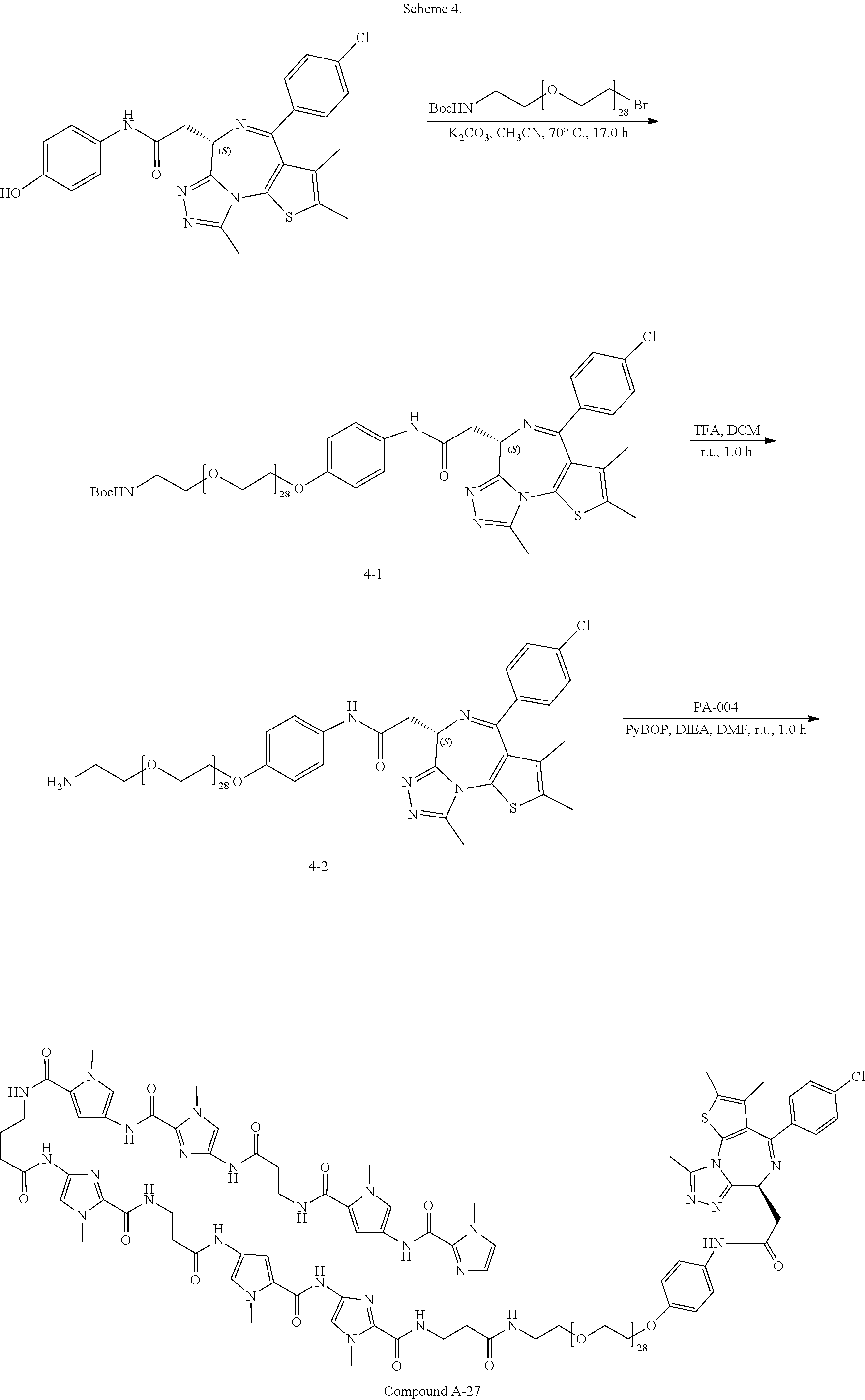

### Step 1. Synthesis of Intermediate 4-1

To a stirred solution of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide (27.00 mg, 0.05 mmol, 1.00 equiv) in CH₃CN (1.50 mL) were added tert-butyl N-(86-bromo-3,6,9,12,15,18,21,24,27,30,33,36,39,42,45,48,51,54,57,60,63,66,69,72,75,78,81,84-octacosaoxahexaoctacontan-1-yl)carbamate (80.00 mg, 0.05 mmol, 1.00 equiv) and K₂CO₃ (22.76 mg, 0.16 mmol, 3.00 equiv). The resulting mixture was stirred for 17.0 h at 70° C. The resulting mixture was filtered, the filter cake was washed with EtOAc (3×8 mL). The filtrate was concentrated under reduced pressure and purified by TLC-plate (CH₂Cl₂/MeOH=8:1) to afford tert-butyl (S)-(86-(4-(2-(4-(4-chlorophenyl)-2,3,9-trimethyl-6H-thieno[3,2-f][1,2,4]triazolo[4,3-a][1,4]diazepin-6-yl)acetamido)phenoxy)3,6,9,12,15,18,21,24,27,30,33,36,39,42,45,48,51,54,57,60,63,66,69,72,75,78,81,84-octacosaoxahexaoctacontyl)carbamate (81.00 mg, 77.40% yield) as a brown solid.

LC/MS: mass calcd. For C₈₈H₁₄₇ClN₆O₃₂S: 1866.95, found: 623.90[M/3+H]⁺.

### Step 2. Synthesis of Intermediate 4-2

A solution of tert-butyl (S)-(86-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24,27,30,33,36,39,42,45,48,51,54,57,60,63,66,69,72,75,78,81,84-octacosaoxahexaoctacontyl)carbamate (70.00 mg, 0.04 mmol, 1.00 equiv) and TFA (0.20 mL) in DCM (1.00 mL) was stirred for 1.0 h at room temperature. The resulting mixture was concentrated under reduced pressure. This resulted in (S)—N-(4-((86-amino-3,6,9,12,15,18,21,24,27,30,33,36,39,42,45,48,51,54,57,60,63,66,69,72,75,78,81,84-octacosaoxahexaoctacontyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide (70.00 mg, crude) as a yellow oil.

LC/MS: mass calcd. For C₈₃H₁₃₉ClN₆O₃₀S: 1766.89, found: 590.60[M/3+H]⁺.

### Step 3. Synthesis of A-26

To a stirred solution of 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid (37.80 mg, 0.03 mmol, 1.00 equiv) in DMF (1.00 mL) was added DIEA (25.27 mg, 0.19 mmol, 6.00 equiv), (S)—N-(4-((86-amino-3,6,9,12,15,18,21,24,27,30,33,36,39,42,45,48,51,54,57,60,63,66,69,72,75,78,81,84-octacosaoxahexaoctacontyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide (57.62 mg, 0.03 mmol, 1.00 equiv) and PyBOP (25.43 mg, 0.05 mmol, 1.50 equiv) at 0° C. The resulting mixture was stirred for 1.0 h at room temperature. The resulting mixture was filtered and purified by Perp-HPLC with the following condonation: Column: XBridge Prep C18 OBD Column, 19*150 mm, 5 μm; Mobile Phase A: Water (10 mmol/L NH₄HCO₃), Mobile Phase B: ACN; Flow rate: 25 mL/min; Gradient: 45% B to 65% B in 11 min, 65% B; Wave Length: 254 nm; RT1 (min): 3.05; Number Of Runs: 0. The fractions were combined and lyophilized to afford (S)—N-(3-((5-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-88-oxo-3,6,9,12,15,18,21,24,27,30,33,36,39,42,45,48,51,54,57,60,63,66,69,72,75,78,81,84-octacosaoxa-87-azanonacontan-90-yl)carbamoyl)-1-methyl-1H-imidazol-4-yl)carbamoyl)-1-methyl-1H-pyrrol-3-yl)amino)-3-oxopropyl)-1-methyl-4-(4-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)butanamido)-1H-imidazole-2-carboxamide (13.80 mg, 14.46%) as a white solid. HRMS: mass calcd. For C₁₃₄H₁₉₈ClN₂₇O₄₁S: 2908.3648, found: 2909.3729 [M+H]⁺.

HPLC: 99.401% purity.

### Example 9. General Synthesis and Purification of the Compounds of the Disclosure

Compounds of the disclosure were made by methods similar to Example 4. The compounds were subsequently purified by HRMS methods A or B.

Method A: Instrument: Waters Acquity I Class UPLC with Xevo G2-XSQ Tof HRMS; Column: ACQUITY UPLC BEH-C18, 2.1×50 mm, 2.7 m; mobile phase A: H₂O (0.1% HCOOH), mobile B, CAN (0.1% HCOOH); Flow rate: 0.4 mL/min; Gradient: 10% B to 95% B in 1.5 min, hold 95% for another 0.5 min, then down to 10% B in 0.3 min, hold 10% B for another 0.7 min; detector: 254 nm.

Method B: Instrument: Waters Acquity I Class UPLC with Xevo G2-XS Q Tof HRMS; Column: ACQUITY UPLC BEH-C18, 2.1×50 mm, 2.7 μm; mobile phase A: H₂O (0.1% HCOOH), mobile B, CAN (0.1% A HCOOH); Flow rate: 0.4 m/min; Gradient: 5% B to 4% B in 2.0 min, to 95% in another 1.5 m, hold 95% for 1.5 min, then down to 5% B in 0.3 min, hold 5% B for another 0.7 min; detector: 254 nm.

Experimental data for the compounds of the disclosure purified by Method A are provided in Table 5.

TABLE 5
LCMS analysis of compounds of the disclosure.

| Comp. No. | Exact Mass | Free Base MW | Mass Found |
|---|---|---|---|
| A-1 | 2027.8405 | 2029.656 | 2028.8478 |
| A-2 | 2410.0176 | 2411.5808 | 2411.0159 |
| A-3 | 2035.8435 | 2037.1754 | 2036.8448 |
| A-4 | 2027.8405 | 2029.656 | 2028.8377 |
| A-5 | 2130.8607 | 2132.2518 | 2131.8592 |
| A-6 | 2332.0468 | 2333.977 | 2333.0514 |
| A-7 | 2091.9392 | 2093.2684 | 2092.9497 |
| A-8 | 1939.7881 | 1941.55 | 1940.788 |
| A-9 | 1851.7356 | 1853.444 | 1852.7413 |
| A-10 | 1763.6832 | 1765.338 | 1764.6895 |
| A-11 | 1675.6308 | 1677.232 | 1676.6254 |
| A-12 | 2101.8341 | 2103.2098 | 2102.831 |
| A-14 | 1954.7558 | 1956.0398 | 1955.7612 |
| A-15 | 2264.9869 | 2266.948 | 2265.9864 |
| A-16 | 2264.9869 | 2266.948 | 2265.9857 |
| A-17 | 2336.024 | 2338.027 | 2337.0146 |
| A-18 | 2374.1171 | 2375.633 | 2375.1068 |
| A-19 | 2414.1383 | 2415.6664 | 2415.1287 |
| A-20 | 2449.0849 | 2450.6864 | 2450.0921 |
| A-21 | 1880.7541 | 1881.9938 | 1881.7648 |
| A-22 | 2203.9453 | 2205.868 | 2204.9598 |
| A-23 | 2468.1026 | 2470.186 | 2469.0977 |
| A-24 | 2556.1551 | 2558.292 | 2557.1534 |
| A-26 | 2776.2861 | 2778.557 | 2777.2906 |
| A-27 | 2908.3648 | 2910.716 | 2909.3729 |
| A-28 | 2996.4172 | 2998.822 | 2997.4188 |
| A-29 | 2302.025 | 2303.947 | 2303.0312 |
| A-30 | 2273.1382 | 2274.573 | 2274.1414 |
| A-32 | 2302.107 | 2303.5354 | 2303.109 |
| A-33 | 2272.0964 | 2273.5094 | 2273.1027 |
| A-35 | 1777.7339 | 1779.398 | 1778.7456 |
| A-36 | 2188.9376 | 2190.3648 | 2189.9359 |
| A-37 | 1777.7339 | 1779.398 | 1778.7411 |
| A-38 | 2085.9174 | 2087.769 | 2086.9287 |
| A-39 | 1880.7541 | 1881.9938 | 1881.7619 |
| A-40 | 2188.9376 | 2190.3648 | 2189.9468 |
| A-41 | 2480.0959 | 2481.7158 | 2481.0976 |
| A-42 | 2414.1053 | 2415.6814 | 2415.1071 |
| A-43 | 2334.1321 | 2335.605 | 2335.1469 |
| A-44 | 2025.9486 | 2027.234 | 2026.955 |
| A-45 | 2336.024 | 2338.027 | 2359.0190[M + Na] |
| A-46 | 2207.9654 | 2209.896 | 2208.9786 |
| A-47 | 1722.6916 | 1724.709 | 1723.709 |
| A-48 | 1898.7964 | 1900.921 | 1899.8109 |
| A-49 | 2206.9799 | 2209.292 | 2207.9987 |
| A-50 | 2438.0489 | 2439.6348 | 2439.0492 |
| A-51 | 2370.0791 | 2371.6284 | 2373.0673 |
| A-52 | 1854.8258 | 1856.47 | 1855.8324 |
| A-53 | 2030.9307 | 2032.682 | 2031.9369 |
| A-54 | 2339.1142 | 2341.053 | 2340.1233 |
| A-55 | 1859.7387 | 1860.9634 | 1860.7429 |
| A-56 | 2216.0803 | 2217.477 | 2217.0815 |
| A-57 | 1907.8968 | 1909.106 | 1908.8999 |
| A-58 | 2308.1065 | 2309.574 | 2309.1194 |
| A-59 | 1999.923 | 2001.203 | 2000.9344 |
| A-60 | 2024.9646 | 2026.25 | 1941.9203 |
| A-61 | 2333.1481 | 2334.621 | 2250.0935 |
| A-62 | 2344.0270 | 2345.5464 | 2345.0348 |
| A-65 | 2373.0536 | 2374.5884 | 2374.0653 |
| A-66 | 2240.9750 | 2242.4294 | 2241.9809 |
| A-67 | 2152.9225 | 2154.3234 | 2153.9327 |
| A-68 | 2302.0165 | 2303.5094 | 2303.0264 |
| A-69 | 2169.9378 | 2171.3504 | 2170.9403 |
| A-70 | 2081.8854 | 2083.2444 | 2082.8987 |
| A-71 | 2309.0131 | 2311.0010 | 2310.0186 |
| A-72 | 2441.0234 | 2442.5948 | 2442.0414 |
| A-73 | 2338.0033 | 2339.9990 | 2339.0168 |
| A-74 | 2440.0394 | 2441.6108 | 2441.0594 |
| A-75 | 2337.0192 | 2339.0150 | 2338.0347 |

### BIOLOGICAL EXAMPLES

### Example B1. Activity

Fibroblast: a cell type derived from a skin biopsy of a patient. These cells are not altered genetically, so they serve as a primary cell culture model of disease

iPSC: induced pluripotent stem cell, a cell type that results as a reprogramming of another cell type (typically skin cells or blood cells) into a more embryonic-like state that enables the development of other cell types to model therapeutic effects of drugs in vitro.

SNP: Single Nucleotide Polymorphism, a variation in a single base pair in a DNA sequence

Molecular Biology Toolkit:
qPCR primer probe sets:
   RNA input normalization was assessed utilizing human glyceraldehyde 3-phosphate dehydrogenase (hGAPDH) TaqMan assay (ThermoFisher cat #4351370) or Human Cyclophillin (IAPP) TaqMan assay (ThermoFisher cat #4351372)
   Total HTT detection was assessed utilizing human Htt TaqMan assay (ThermoFisher cat #4331182)
   Allele-specific detection of human HTT expression in HD cells containing the SNP rs362331C/T (Exon 50): for each assay, allele-specific probes to detect the SNP variant contained locked nucleic acid bases to improve allele discrimination, as compared to unmodified DNA probes.

362331-F (331 forward primer):
TCTCCTCCACAGAGTTTGTGA
362331-R (331 reverse primer):
CCTTCTTTCTGGACTAAGAAGCTG
362331-C probe:
TCC CTC ATC + C + AC TGT GT
362331-T probe:
CTC + A + T + C + T + A + C TGT GT

qPCR was performed using Agpath ID one-step reverse transcriptase polymerase chain reaction (RT-PCR) reagent

Protein measurements were performed via western blots probing with antibody MW1 (polyQ specific) to assess reduction of mtHTT alone. D7F7 (a.a surrounding Pro1218) was used to visualize both wtHTT and mtHTT. Lysates were standardized by DC prior to separation on a 3-8% Tris-acetate gel and transferred via wet transfer method onto nitrocellulose membranes. Blots were probed with the previously mentioned antibodies and complementary fluorescent secondary antibodies and imaged on the Li-Cor Odyssey® DLx Imaging system.

Antibody pairing of 2B7 (a.a. 1-17) and MW1 (polyQ specific) will be used to track mtHTT levels while pairing of MAB2166 (a.a. 181-810) and MAB5490 (a.a. 115-129) will be employed to track total full-length HTT.

Screening of HD molecules methods: GM09197 and/or GM04022 fibroblasts were cultured in T175 flasks incubated at 37° C. and 5% CO₂. Once confluency was reached, the media was removed, the cells were washed with 1×PBS, and cells were dissociated using TrypLE™ Express Enzyme. Media was added to the enzyme and collected into a 15-mL conical tube and centrifuged at 500×g for 5 minutes to pellet the cells. Media and enzyme were aspirated using a serological pipette. Cells were resuspended in fresh media and counted using a Countess 3 Automated Cell Counter. Cells were plated at a density of 15,000 cells/well in a tissue culture-treated polystyrene 96-well dish and incubated at 37° C. and 5% CO₂ overnight. The next day, media was removed using an 8-channel aspirator. 200 μL media/well are added back into the plate. The molecules are formulated to 1 mM and are dispensed using a Multidrop™ Pico 8 Digital Dispenser. After a 48-hour incubation with compounds, media was removed from plates, cells were washed with 1×PBS, and cells were lysed in 40 μL per well guanidinium thiocyanate buffer. RNA was isolated and purified in 382-well glass fiber column plates using chaotropic salts. Human mtHTT, wtHTT, and GAPDH mRNA were measured via RT-PCR using the ThermoFisher QuantStudio™ 7 Flex System in 384-well format. Results of HTT levels were normalized to GAPDH mRNA levels. Normalized HTT mRNA levels were expressed relative to vehicle-treated samples to assess fold change after molecule treatment.

iPSC-Neuron Duration of Action of HD molecules methods: Fibroblasts isolated from HD patients were reprogrammed into iPSCs expanded in the presence of cytokines and transduced with the Sendai virus, a cytoplasmic RNA vector. These iPSCs expressed stem cell markers and have normal karyotypes and express the pluripotent markers Nanog, Tra-1-60, and SSeA-44. iPSC-derived neuron differentiation methodology followed standard protocols for mixed cortical neuron differentiation resulting in immunohistochemical staining of iPS-Neuron of Tuj1 and Map2. iPSC-neuronal precursor cells were plated at 300,000 cells/well in a PLO/Laminin-521 coated culture-treated polystyrene 96-well dish and incubated at 37° C. and 5% CO₂. The next day, media was changed to allow neuron precursor cells to continue maturation into neurons. Four days later, media was refreshed, and cells were treated with mitotic inhibitor to remove any remaining dividing cells, resulting in a pure neuronal culture. Three days later, media was removed. 200 μL media/well were added back into the plate. The molecules were formulated to 1 mM and dispensed using a Multidrop™ Pico 8 Digital Dispenser. After 96-hour incubation with compounds, media was removed and refreshed and the cells were retreated. After 7 days of compound exposure, media was removed from plates, and cells were lysed in 60 μL of Ambion Lysis buffer. RNA is isolated using PureLink™ RNA isolation kits. cDNA is synthesized with Agilent Superscript II kit. Human mtHTT, wtHTT, and GAPDH mRNA were measured via RT-PCR using the ThermoFisher QuantStudio™ 7 Flex System in 384-well format. Results of HTT levels were normalized to GAPDH mRNA levels. Normalized HTT mRNA levels were expressed relative to vehicle-treated samples to assess fold change after treatment with the HD compounds.

Eₘᵢₙ for each compound is the lowest % HTT concentration observed within a compound concentration of 0.5 nM to 1000 nM.

Representative in vitro biochemical data is presented in Table 6 and Table 7, where A>90%, B is 90% to 80%, C<80%.

TABLE 6
Representative biochemical data in GM09197 fibroblasts.

| | GM09197 IC₅₀ | GM 09197 E | GM09197 | Gm09197 Emin |
|---|---|---|---|---|
| Comp. | 48 hr C Allele | min 48 Hr C | IC₅₀ 48 hr T | 48 hr T Allele |
| No. | (nM) | Allele (%) | Allele (nM) | (%) |
| A-1 | — | A | 113.637 | C |
| A-2 | — | A | 31.952 | C |
| A-3 | — | A | 38.667 | C |
| A-5 | — | A | 9.8 | C |
| A-7 | — | A | >1000 | A |
| A-20 | >1000 | A | >1000 | A |
| A-21 | >1000 | A | >1000 | A |
| A-29 | >1000 | A | >1000 | B |
| A-30 | >1000 | A | 104.895 | C |
| A-31 | >1000 | B | >1000 | B |
| A-32 | >1000 | B | >1000 | B |
| A-35 | >1000 | A | >1000 | B |
| A-36 | >1000 | A | >1000 | A |
| A-37 | >1000 | B | >1000 | B |
| A-38 | >1000 | A | >1000 | A |
| A-39 | >1000 | B | >1000 | B |
| A-40 | >1000 | A | >1000 | A |
| A-45 | >1000 | C | >1000 | C |
| A-46 | >1000 | B | >1000 | A |
| A-47 | >1000 | C | >1000 | C |
| A-48 | >1000 | A | >1000 | A |
| A-49 | >1000 | A | >1000 | A |
| A-52 | >1000 | C | >1000 | B |
| A-53 | >1000 | A | >1000 | B |
| A-54 | >1000 | B | >1000 | B |
| A-55 | >1000 | A | >1000 | A |
| A-56 | >1000 | C | 104 | C |
| A-58 | >1000 | B | >1000 | B |
| A-59 | >1000 | B | >1000 | B |
| A-68 | — | A | 17 | C |
| A-71 | >1000 | A | >1000 | A |
| A-72 | >1000 | B | >1000 | A |
| A-73 | >1000 | A | >1000 | A |
| A-74 | >1000 | B | >1000 | A |
| A-75 | >1000 | A | >1000 | A |

TABLE 7
Representative biochemical data in GM04022 fibroblasts.

| | | | GM04022 | GM04022 |
|---|---|---|---|---|
| Comp. | GM04022 IC₅₀ | GM 04022Emin | IC₅₀ 48 hr | Emin 48 hr |
| No. | 48 hr C allele | 48 hr C Allele | T Allele | T Allele |
| A-2 | — | A | >1000 | C |
| A-3 | — | A | 36 | C |
| A-5 | — | A | 2 | C |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A transcription modulator molecule having a first terminus, a second terminus, and an oligomeric backbone moiety, wherein:
a) the first terminus comprises a moiety having the structure of Formula (A-1): 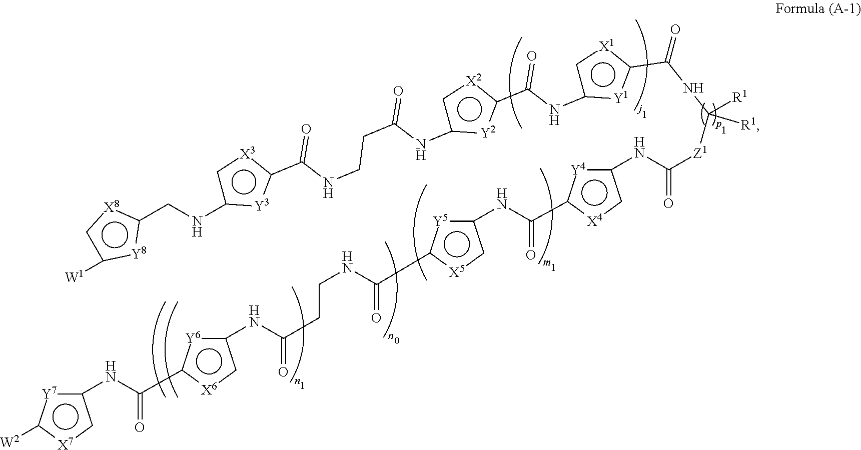
or a pharmaceutically acceptable salt thereof, wherein:
Z¹ is absent, —O—, or —NH—;
each X¹, X², X³, X⁴, X⁵, X⁶, X⁷, and X⁸ is independently 0, S, or NR²;
each Y¹, Y Y³, Y⁴, Y⁵, Y⁶, Y⁷, and Y⁸ is independently CH or N;
W¹ is deuterium, hydrogen, optionally substituted C₁-C₆ alkyl, (azaneylidene)methanediamine, (azaneylidene)-N,N,N′,N′-tetramethylmethanediamine, —C(O)—NR^{1A}R^{1B}, —NR^{1A}—C(O)—NR^{1A}R^{1B}, —Z_{B}—P(O)(OR^{1A})₂, —Z_{B}—(CH₂)ₚ—P(O)(OR^{1A})₂, —Z_{B}—(CH₂)ₚ₃—O—P(O)(OR^{1A})₂, wherein
Z_{B} is —N— or —O—;
p₃ is an integer from 1-10;
W² is an optionally substituted C₁-C₆ alkyl or —C(O)—NR^{1A}R^{1B};
each R¹ is independently hydrogen, deuterium, halogen, amino, cyano, optionally C₁-C₂₀ alkyl, or C₁-C₂₀ heteroalkyl, or —NHC(O)R^{1A}; or
two R¹ on the same or on adjacent atoms combine together with the atom(s) to which they are attached to form an optionally substituted 3 to 6-membered carbocyclic ring or 3 to 6-membered heterocyclic ring;
each R² is independently hydrogen, deuterium, optionally substituted C₁-C₂₀ alkyl, optionally substituted C₁-C₂₀ heteroalkyl, optionally substituted C₁-C₂₀ haloalkyl, or optionally substituted C₁-C₂₀ alkylamino;
each R^{1A} is independently hydrogen, deuterium, or optionally substituted C₁-C₂₀ alkyl;
each R^{1B} is independently hydrogen, deuterium, optionally substituted 5-membered heteroaryl, optionally substituted C₁-C₂₀ alkyl, optionally substituted C₂-C₁₀ heteroalkyl, or (AA)ₚ₂, wherein
each AA is an amino acid;
j₂ is an integer from 1-10;
j₁ is 0 or 1;
n⁰ is 0 or 1;
m₁ and n₁ are each independently an integer from 0-3; and
p₁ is 2 or 3, provided that when Z¹ is O or NH then p₁ is 2, and when Z¹ is absent then p₁ is 3;
wherein one of W¹ or W² is attached to the oligomeric backbone;
b) the second terminus comprises a moiety capable of binding to a regulatory molecule that modulates expression of a gene by binding a target selected from a member of the BET (bromodomain and extra-terminal) family, CBP/p300, PCAF (P300/CBP-Associated Factor), CECR2 (cat eye syndrome chromosome region candidate 2), BRPF (bromodomain and PHD finger-containing protein), ATAD2/ATAD2B (chromatin remodeling proteins), TRIM24 (Tripartite motif-containing 24), BAZ2 (Bromodomain Adjacent to Zinc finger), or TAF1 (TBP associated factors); and
c) the oligomeric backbone links the first terminus and the second terminus.

2. The molecule of claim 1, or a pharmaceutically acceptable salt thereof, wherein the first terminus comprises the structure of Formula (A-4), or a pharmaceutically acceptable salt thereof: 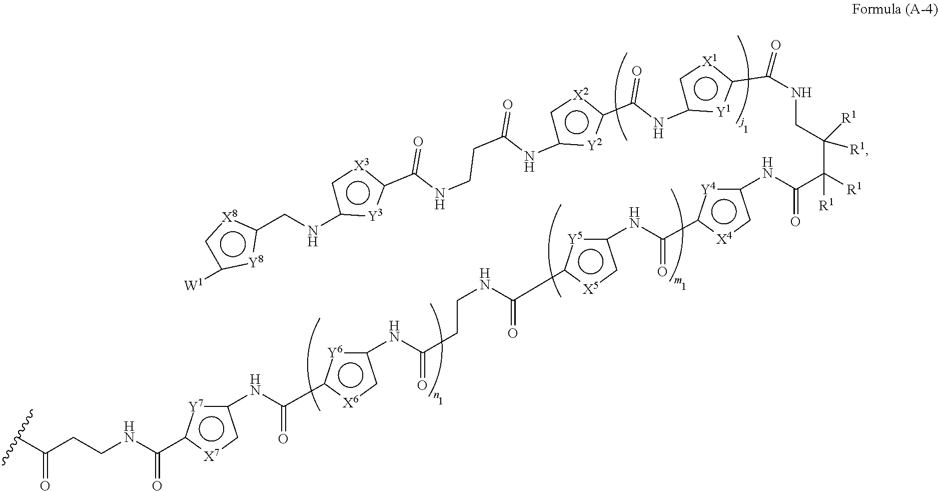
wherein:
each X¹, X², X³, X⁴, X⁵, X⁶, X⁷, and X⁸ is independently O, S, or NR²;
each Y¹, Y², Y³, Y⁴, Y⁵, Y⁶, Y⁷, and Y⁸ is independently CH or N;
W¹ is deuterium, hydrogen, optionally substituted C₁-C₆ alkyl, (azaneylidene)methanediamine, (azaneylidene)-N,N,N′,N′-tetramethylmethanediamine, —C(O)—NR^{1A}R^{1B}, —NR^{1A}—C(O)—NR^{1A}R^{1B}, —Z_{B}—P(O)(OR^{1A})₂, —Z_{B}—(CH₂)ₚ—P(O)(OR^{1A})₂, —Z_{B}—(CH₂)ₚ₃—O—P(O)(OR^{1A})₂, wherein
Z_{B} is —N— or —O—;
p₃ is an integer from 1-10;
each R¹ is independently hydrogen, deuterium, halogen, amino, cyano, optionally C₁-C₂₀ alkyl, or C₁-C₂₀ heteroalkyl, or —NHC(O)R^{1A}; or
two R¹ on the same or on adjacent atoms combine together with the atom(s) to which they are attached to form an optionally substituted 3 to 6-membered carbocyclic ring or 3 to 6-membered heterocyclic ring;
each R² is independently hydrogen, deuterium, optionally substituted C₁-C₂₀ alkyl, optionally substituted C₁-C₂₀ heteroalkyl, optionally substituted C₁-C₂₀ haloalkyl, or optionally substituted C₁-C₂₀ alkylamino;
each R^{1A} is independently hydrogen, deuterium, or optionally substituted C₁-C₂₀ alkyl;
each R^{1B} is independently hydrogen, deuterium, optionally substituted 5-membered heteroaryl, optionally substituted C₁-C₂₀ alkyl, optionally substituted C₂-C₁₀ heteroalkyl, or (AA)ₚ₂, wherein
each AA is an amino acid;
p₂ is an integer from 1-10;
j₁ is 0 or 1; and
m₁ and n₁ are each independently an integer from 0-3.

3. The molecule of claim 1, or a pharmaceutically acceptable salt thereof, wherein X¹, X², X³, X⁴, X⁵, X⁶, and X⁷ are each independently —NR².

4. The molecule of claim 1, or a pharmaceutically acceptable salt thereof, wherein each Y², Y⁴, and Y⁷ are each independently N; and each Y¹ and Y³ are independently CH.

5. The molecule of claim 1, or a pharmaceutically acceptable salt thereof, wherein each Y⁶ is independently CH.

6. The molecule of claim 1, or a pharmaceutically acceptable salt thereof, wherein n₁ is 0.

7. The molecule of claim 1, or a pharmaceutically acceptable salt thereof, wherein the oligomeric backbone is a linker having a length of less than about 50 Angstroms.

8. The molecule of claim 1, or a pharmaceutically acceptable salt thereof, wherein the oligomeric backbone comprises a multimer having 2 to 50 spacing moieties, wherein
each spacing moiety is independently selected from the group consisting of —((CR^{3a}R^{3b})ₓ—O)_{y}—, —((CR^{3a}R^{3b})ₓ—NR^{4a})_{y}—, —((CR^{3a}R^{3b})ₓ—CH═CH—(CR^{3a}R^{3b})ₓ—O)_{y}—, optionally substituted C₁-C₁₂ alkyl, optionally substituted C₂-C₁₀ alkenyl, optionally substituted C₂-C₁₀ alkynyl, optionally substituted C₆-C₁₀ arylene, optionally substituted C₃-C₇ cycloalkylene, optionally substituted 5 to 10-membered heteroarylene, optionally substituted 4 to 10-membered heterocycloalkylene, amino acid residue, —O—, —C(O)NR^{4a}—, —NR^{4a}C(O)—, —C(O)—, —NR^{1a}—, —C(O)O—, —S—, —S(O)—, —S(O)₂—, —S(O)₂NR^{4a}—, —NR^{4a}S(O)₂—, and —P(O)OH—, and any combinations thereof, wherein
each x is independently 2-4;
each y is independently 1-10;
each R^{1a} is independently a hydrogen or optionally substituted C₁-C₆ alkyl;
each R^{3a} and R^{3b} is independently selected from hydrogen, optionally substituted alkyl, optionally substituted alkenyl, optionally substituted alkynyl, optionally substituted alkoxy, optionally substituted amino, carboxyl, carboxyl ester, acyl, acyloxy, acyl amino, amino acyl, optionally substituted alkylamide, sulfonyl, optionally substituted thioalkoxy, optionally substituted aryl, optionally substituted heteroaryl, optionally substituted cycloalkyl, and optionally substituted heterocyclyl; and
each R^{4a} is independently a hydrogen or an optionally substituted C₁-C₆ alkyl.

9. The molecule of claim 1, or a pharmaceutically acceptable salt thereof, wherein the second terminus comprises a CBP/p300 binding moiety.

10. The molecule of claim 1, or a pharmaceutically acceptable salt thereof, wherein the second terminus comprises a PCAF (P300/CBP-Associated Factor) binding moiety.

11. The molecule of claim 1, or a pharmaceutically acceptable salt thereof, wherein the second terminus comprises the structure of Formula (4-A): 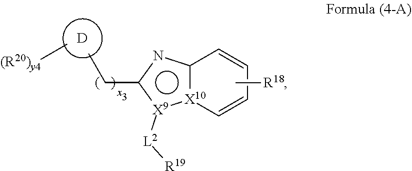
or a pharmaceutically acceptable salt thereof, wherein:
Ring D is absent, phenyl, or 5 to 6-membered heteroaryl;
X⁹ and X¹⁰ are each independently C or N, wherein one of X⁹ or X¹⁰ is N;
L² is absent, optionally substituted alkylene, —O—, or —NR^{D}—, wherein
R^{D} is hydrogen, deuterium, or optionally substituted C₁-C₃ alkyl;
R¹⁸ is an optionally substituted 5 to 6-membered heteroaryl;
R¹⁹ is an optionally substituted C₃-C₅ cycloalkyl or optionally substituted 4 to 7-membered heteroaryl;
each R²⁰ is independently hydrogen, deuterium, halogen, —CN, —NO₂, optionally substituted C₁-C₆ alkyl, optionally substituted C₁-C₆ haloalkyl, or optionally substituted C₁-C₆ hydroxyalkyl;
x₃ is an integer from 1-3;
y₄ is an integer from 1-4; and
wherein the attachment to the linker is at either R¹⁹ or at one of R²⁰.

12. The molecule of claim 1, or a pharmaceutically acceptable salt thereof, wherein the second terminus comprises the structure of Formula (4-B): 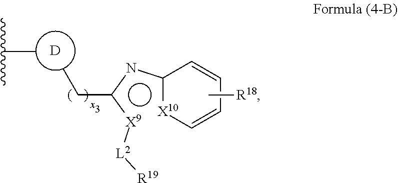
or a pharmaceutically acceptable salt thereof, wherein:
Ring D is absent, optionally substituted phenyl, or optionally substituted 5 to 6-membered heteroaryl;
X⁹ and X¹⁰ are each independently C or N, wherein one of X⁹ or X¹⁰ is N;
L² is absent, optionally substituted alkylene, —O—, or —NR^{D}—, wherein
R^{D} is hydrogen, deuterium, or optionally substituted C₁-C₃ alkyl;
R¹⁸ is an optionally substituted 5 to 6-membered heteroaryl;
R¹⁹ is an optionally substituted C₃-C₅ cycloalkyl or optionally substituted 4 to 7-membered heteroaryl; and
x₃ is an integer from 1-3.

13. The molecule of claim 1, or a pharmaceutically acceptable salt thereof, wherein the second terminus comprises the structure of Formula (4-D): 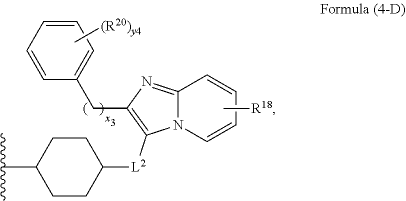
or a pharmaceutically acceptable salt thereof, wherein:
L² is an optionally substituted alkylene, —O—, or —NR^{D}—, wherein
R^{D} is hydrogen, deuterium, or optionally substituted C₁-C₃ alkyl;
R¹⁸ is an optionally substituted 5 to 6-membered heteroaryl;
R²⁰ is hydrogen, deuterium, halogen, —CN, —NO₂, optionally substituted C₁-C₆ alkyl, optionally substituted C₁-C₆ haloalkyl, or optionally substituted C₁-C₆ hydroxyalkyl;
x₃ is an integer from 1-3; and
y₄ is an integer from 1-4.

14. The molecule of claim 1, or a pharmaceutically acceptable salt thereof, wherein the second terminus comprises the structure of Formula (7-A): 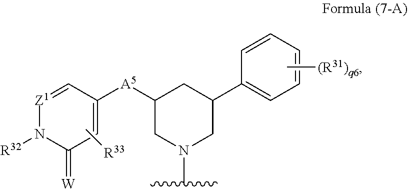
or a pharmaceutically acceptable salt thereof, wherein:
A⁵ is —O—, —NH—, or —CH₂—;
Z¹ is CH or N;
W is O or S;
each R³¹ is independently hydrogen, halogen, —OH, —CN, —NO₂, —NH₂, optionally substituted C₁-C₁₀ alkyl, optionally substituted C₁-C₁₀ haloalkyl, optionally substituted C₁-C₁₀ hydroxyalkyl, optionally substituted C₂-C₁₀ alkenyl, optionally substituted C₂-C₁₀ alkynyl, optionally substituted C₃-C₅ cycloalkyl, or optionally substituted 3 to 8-membered heterocycloalkyl;
or two R³¹ together with the atoms to which they are attached join together to form an optionally substituted C₅-C₈ cycloalkyl or optionally substituted 5 to 8-membered heterocycloalkyl;
R³² is hydrogen or optionally substituted C₁-C₁₀ alkyl;
R³³ is hydrogen, halogen, —OH, —CN, —NO₂, —NH₂, optionally substituted C₁-C₁₀ alkyl, optionally substituted C₁-C₁₀ haloalkyl, or optionally substituted C₁-C₁₀ hydroxyalkyl; and
q₆ is 0-4.

15. The molecule of claim 1, wherein the second terminus is selected from: 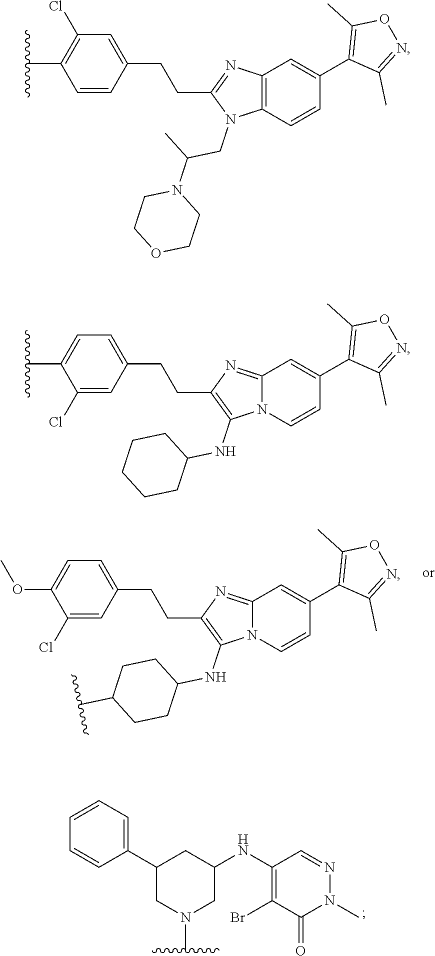 [SMILES: *c1ccc(CCc2nc3cc(-c4c(C)noc4C)ccc3n2CC(C)N2CCOCC2)cc1Cl ; *c1ccc(CCc2nc3cc(-c4c(C)noc4C)ccn3c2NC2CCCCC2)cc1Cl ; *C1CCC(Nc2c(CCc3ccc(OC)c(Cl)c3)nc3cc(-c4c(C)noc4C)ccn23)CC1 ; "or" ; *N1CC(Nc2cnn(C)c(=O)c2Br)CC(c2ccccc2)C1]
or a pharmaceutically acceptable salt thereof.

16. The molecule of claim 1, wherein the second terminus is selected from: 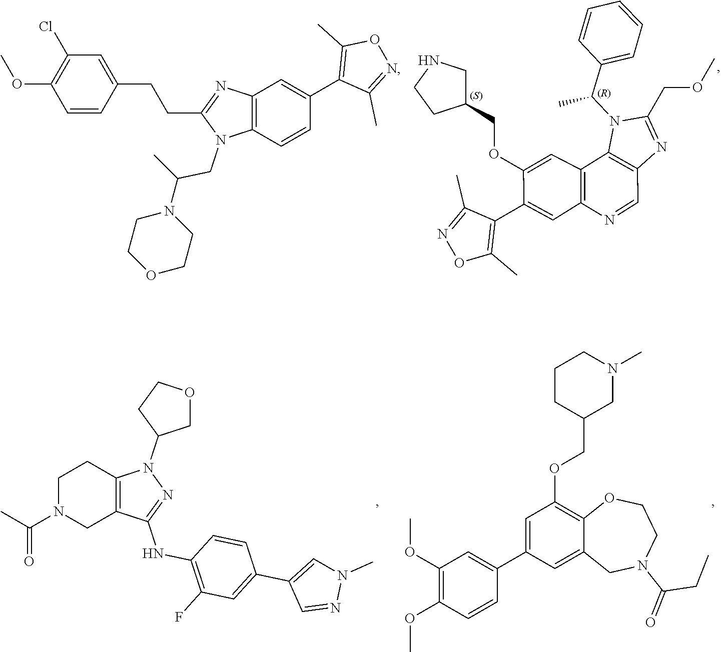 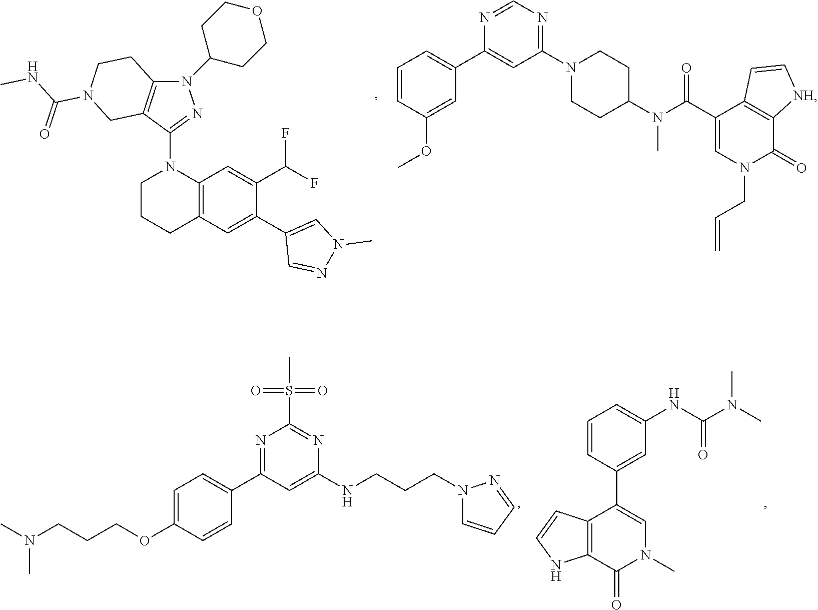 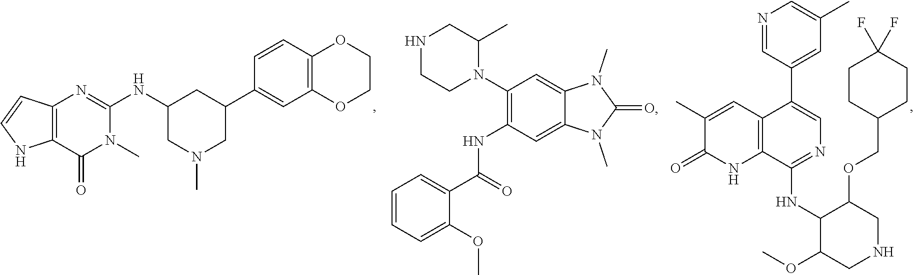 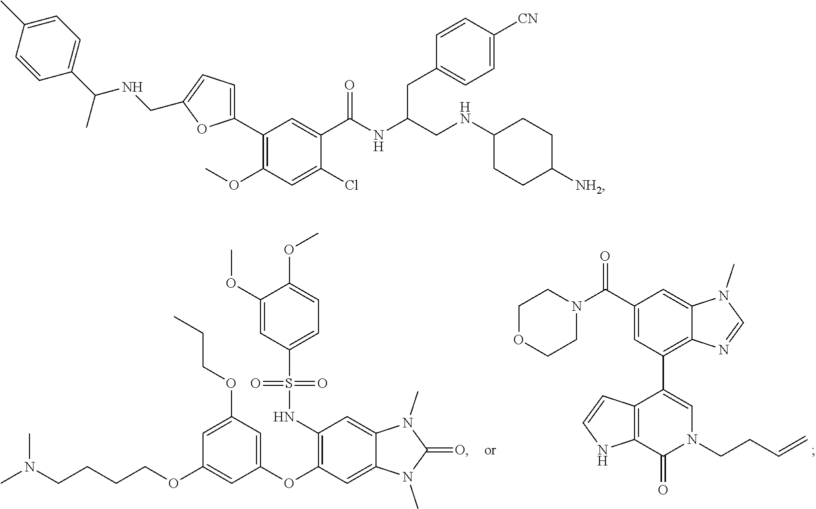 [SMILES: COc1cc(Cl)c(C(=O)NC(CNC2CCC(N)CC2)Cc2ccc(C#N)cc2)cc1-c1ccc(CNC(C)c2ccc(C)cc2)o1 ; CCCOc1cc(OCCCCN(C)C)cc(Oc2cc3c(cc2NS(=O)(=O)c2ccc(OC)c(OC)c2)n(C)c(=O)n3C)c1 ; C=CCCn1cc(-c2cc(C(=O)N3CCOCC3)cc3c2ncn3C)c2cc[nH]c2c1=O]
or a pharmaceutically acceptable salt thereof.

17. A method of modulating a gene associated with transcription comprising binding a transcription modulator molecule having a first terminus, a second terminus, and an oligomeric backbone, wherein:
a) the first terminus comprises a DNA-binding moiety capable of binding a nucleotide repeat;
b) the second terminus comprises a binding moiety capable of binding to a regulatory molecule that modulates expression of a gene by binding a target selected from a member of the BET (bromodomain and extra-terminal) family, CBP/p300, PCAF (P300/CBP-Associated Factor), CECR2 (cat eye syndrome chromosome region candidate 2), BRPF (bromodomain and PHD finger-containing protein), ATAD2/ATAD2B (chromatin remodeling proteins), TRIM24 (Tripartite motif-containing 24), BAZ2 (Bromodomain Adjacent to Zinc finger), or TAF1 (TBP associated factors); and
c) the oligomeric backbone links the first terminus and the second terminus.

18. The method of claim 17, wherein the DNA-binding moiety comprises a structure of Formula (A-1): 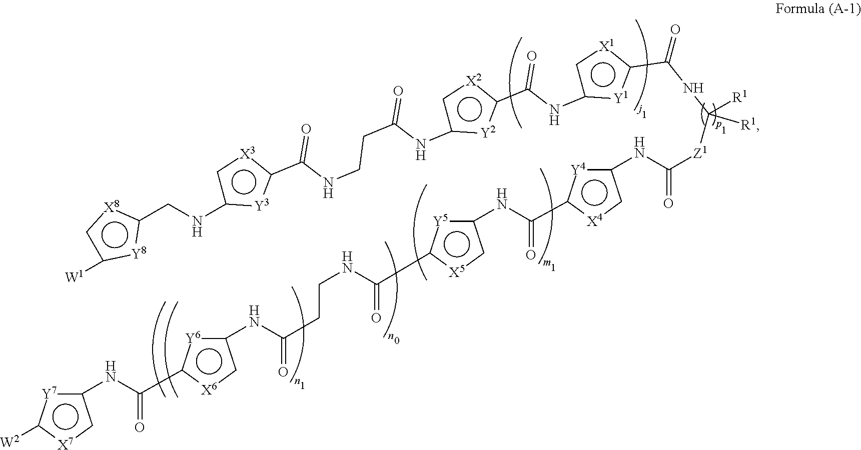
or a pharmaceutically acceptable salt thereof, wherein:
Z¹ is absent, —O—, or —NH—;
each X¹, X², X³, X⁴, X⁵, X⁶, X⁷, and X⁸ is independently 0, S, or NR²;
each Y¹, Y², Y³, Y⁴, Y⁵, Y⁶, Y⁷, and Y⁸ is independently CH or N;
W¹ is deuterium, hydrogen, optionally substituted C₁-C₆ alkyl, (azaneylidene)methanediamine, (azaneylidene)-N,N,N′,N′-tetramethylmethanediamine, —C(O)—NR^{1A}R^{1B}, —NR^{1A}—C(O)—NR^{1A}R^{1B}, —Z_{B}—P(O)(OR^{1A})₂, —Z_{B}—(CH₂)ₚ—P(O)(OR^{1A})₂, —Z_{B}—(CH₂)ₚ₃—O—P(O)(OR^{1A})₂, wherein
Z_{B} is —N— or —O—;
p₃ is an integer from 1-10;
W² is an optionally substituted C₁-C₆ alkyl or —C(O)—NR^{1A}R^{1B};
each R¹ is independently hydrogen, deuterium, halogen, amino, cyano, optionally C₁-C₂₀ alkyl, or C₁-C₂₀ heteroalkyl, or —NHC(O)R^{1A}; or
two R¹ on the same or on adjacent atoms combine together with the atom(s) to which they are attached to form an optionally substituted 3 to 6-membered carbocyclic ring or 3 to 6-membered heterocyclic ring;
each R² is independently hydrogen, deuterium, optionally substituted C₁-C₂₀ alkyl, optionally substituted C₁-C₂₀ heteroalkyl, optionally substituted C₁-C₂₀ haloalkyl, or optionally substituted C₁-C₂₀ alkylamino;
each R^{1A} is independently hydrogen, deuterium, or optionally substituted C₁-C₂₀ alkyl;
each R^{1B} is independently hydrogen, deuterium, optionally substituted 5-membered heteroaryl, optionally substituted C₁-C₂₀ alkyl, optionally substituted C₂-C₁₀ heteroalkyl, or (AA)ₚ₂, wherein
each AA is an amino acid;
p₂ is an integer from 1-10;
j₁ is 0 or 1;
n₀ is 0 or 1;
m₁ and n₁ are each independently an integer from 0-3; and
p₁ is 2 or 3, provided that when Z¹ is O or NH then p₁ is 2, and when Z¹ is absent then p₁ is 3;
wherein one of W¹ or W² is attached to the oligomeric backbone.

19. The molecule of claim 17, or a pharmaceutically acceptable salt thereof, wherein the second terminus comprises a CBP/p300 binding moiety.

20. The molecule of claim 17, or a pharmaceutically acceptable salt thereof, wherein the second terminus comprises a PCAF (P300/CBP-Associated Factor) binding moiety.

21. The method of claim 17, wherein the gene is huntingtin (HTT).

22. The method of claim 17, wherein the method reduces one or more symptoms of Huntington's disease.
